(19) United States
(12) Patent Application Publication
(10) Pub. No.: US 2022/0243195 A1
(43) Pub. Date: Aug. 4, 2022

(54) BARCODE-BASED NUCLEIC ACID SEQUENCE ASSEMBLY

(71) Applicant: Twist Bioscience Corporation, South San Francisco, CA (US)

(72) Inventors: Rebecca NUGENT, San Francisco, CA (US); Siyuan CHEN, San Mateo, CA (US); Ross KETTLEBOROUGH, San Francisco, CA (US); Elian LEE, Union City, CA (US); Nathan RAYNARD, San Mateo, CA (US)

(21) Appl. No.: 17/721,680

(22) Filed: Apr. 15, 2022

Related U.S. Application Data
(63) Continuation-in-part of application No. 16/906,555, filed on Jun. 19, 2020, now Pat. No. 11,332,738.
(60) Provisional application No. 62/865,094, filed on Jun. 21, 2019.

(51) Int. Cl. C12N 15/10

(52) U.S. Cl. CPC C12N 15/1031; C12N 15/1068; C12N 15/1065

(57) ABSTRACT

Provided herein are methods, systems, and compositions for efficient nucleic acid assembly. Nucleic acid assembly may comprise assembly of variants comprising paired homology.

### CROSS-REFERENCE

This application is a continuation application of U.S. patent application Ser. No. 16/906,555, filed Jun. 19, 2020, which claims the benefit of U.S. Provisional Application No. 62/865,094, filed Jun. 21, 2019, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created Jul. 20, 2020, is named 44854-783_301_SL and is 1,571 bytes in size.

### BACKGROUND

De novo nucleic acid synthesis is a powerful tool for basic biological research and biotechnology applications. While various methods are known for the synthesis of relatively short fragments of nucleic acids on a small scale, these techniques suffer from scalability, automation, speed, accuracy, and cost. Thus, a need remains for efficient methods of variant nucleic acid assembly.

### BRIEF SUMMARY

Provided herein are methods for nucleic acid assembly, comprising: (a) providing a first plurality of polynucleotides, wherein each polynucleotide of the first plurality of polynucleotides comprises a first terminal region of sequence homology; (b) providing a second plurality of polynucleotides, wherein each polynucleotide of the second plurality of polynucleotides comprises a second terminal region of sequence homology to the first terminal region of sequence homology; and (c) contacting the first plurality of polynucleotides and the second plurality of polynucleotides with a reaction mixture comprising an exonuclease, an endonuclease, a polymerase, and a ligase to assemble a library of nucleic acids, wherein at least 80% of the nucleic acids are each present in the library in an amount within 2× of a mean frequency for each of the nucleic acids in the library. Further provided herein are methods, wherein the first plurality of polynucleotides comprises up to 100 different sequences. Further provided herein are methods, wherein the second plurality of polynucleotides comprises up to 100 different sequences. Further provided herein are methods, wherein at least 10,000 nucleic acids are assembled. Further provided herein are methods, wherein at least 100,000 nucleic acids are assembled. Further provided herein are methods, wherein each polynucleotide of the first plurality of polynucleotides comprises up to 2500 bases in length. Further provided herein are methods, wherein each polynucleotide of the second plurality of polynucleotides comprises up to 2500 bases in length. Further provided herein are methods, wherein the exonuclease is exonuclease III. Further provided herein are methods, wherein the endonuclease is a flap endonuclease. Further provided herein are methods, wherein the flap endonuclease is flap endonuclease 1, exonuclease 1, XPG, Dna2, or GEN1. Further provided herein are methods, wherein the polymerase comprises 5′ to 3′ polymerase activity. Further provided herein are methods, wherein the polymerase is a DNA polymerase. Further provided herein are methods, wherein the ligase catalyzes joining of at least two nucleic acids.

Provided herein are methods for nucleic acid assembly, comprising: de novo synthesizing a first nucleic acid comprising in 5′ to 3′ order: a barcode sequence, a first restriction endonuclease site, a second restriction endonuclease site, and a first hypervariable region sequence; de novo synthesizing a second nucleic acid comprising in 5′ to 3′ order: a first region of any defined length sequence, a self-cleaving peptide sequence, a first complementary region adjacent to a first variable region sequence, and a first variable region sequence; contacting the first nucleic acid and the second nucleic to generate a third nucleic acid; providing a fourth nucleic acid comprising in 5′ to 3′ order: a vector sequence, a second complementary region adjacent to a second variable region sequence, a second variable region sequence, a second hypervariable region sequence, the first restriction endonuclease site, and the barcode sequence; contacting the third nucleic acid and the fourth nucleic acid with a restriction endonuclease; and assembling the third nucleic acid and the fourth nucleic acid using a reaction mixture comprising one or more enzymes. Further provided herein are methods, wherein the first restriction endonuclease site or the second restriction endonuclease site is a Type IIS restriction endonuclease (TIIS-RE) site. Further provided herein are methods, wherein the restriction endonuclease is a Type IIS restriction endonuclease. Further provided herein are methods, wherein the reaction mixture comprises a ligase. Further provided herein are methods, wherein the first hypervariable region sequence and the second hypervariable region sequence each comprises a complementary determining region (CDR). Further provided herein are methods, wherein the CDR is CDR3. Further provided herein are methods, wherein the self-cleaving peptide is P2A. Further provided herein are methods, wherein about 100 variants of the first variable region sequence are synthesized. Further provided herein are methods, wherein about 130 variants of the second variable region sequence are synthesized. Further provided herein are methods further comprising amplifying the nucleic acid with a first primer complementary to a first barcode sequence and a second primer wherein at least 99% of the amplicons have no deletions.

Provided herein are methods for nucleic acid assembly, comprising: de novo synthesizing a first nucleic acid comprising a first variable region sequence; de novo synthesizing a second nucleic acid comprising a second variable region sequence; de novo synthesizing a third nucleic acid comprising in 5′ to 3′ order: a first region of fixed variability sequence, a first region of any defined length sequence, a self-cleaving peptide sequence, a first complementary region adjacent to a first variable region sequence, and a second region of fixed variability sequence; and contacting the first nucleic acid, the second nucleic acid, and the third nucleic acid with a reaction mixture comprising an exonuclease, an endonuclease, a polymerase, and a ligase. Further provided herein are methods, wherein the first variable region sequence or the second variable region sequence is amplified with a hypervariable region sequence. Further provided herein are methods, wherein the hypervariable region sequence comprises a CDR. Further provided herein are methods, wherein the CDR is CDR3. Further provided herein are methods further comprising contacting with sequences comprising one or more regions of any defined length. Further provided herein are methods, wherein about 100 variants of the first variable region sequence are synthesized. Further provided herein are methods, wherein about 130 variants of the second variable region sequence are synthesized. Further provided herein are methods, wherein the self-cleaving peptide is P2A. Further provided herein are methods, wherein the exonuclease is exonuclease III. Further provided herein are methods, wherein the endonuclease is a flap endonuclease. Further provided herein are methods, wherein the flap endonuclease is flap endonuclease 1, exonuclease 1, XPG, Dna2, or GEN1. Further provided herein are methods, wherein the polymerase comprises 5′ to 3′ polymerase activity. Further provided herein are methods, wherein the polymerase is a DNA polymerase Further provided herein are methods, wherein the ligase catalyzes joining of at least two nucleic acids. Further provided herein are methods, wherein the first region of fixed variability sequence and the second region of fixed variability sequence are each about 10 to about 100 base pairs. Further provided herein are methods, wherein the first region of fixed variability sequence and the second region of fixed variability sequence are each about 40 base pairs.

Provided herein are methods for nucleic acid assembly, comprising: providing a first nucleic acid comprising a first region of any defined length sequence; providing a second nucleic acid comprising a second region of any defined length sequence; assembling a third nucleic acid comprising in 5′ to 3′ order: a first complementary region adjacent to a first variable region sequence, a first variable region sequence, and a first hypervariable region sequence; assembling a fourth nucleic acid comprising in 5′ to 3′ order: a second complementary region adjacent to a second variable region sequence, a second variable region sequence, and a second hypervariable region sequence; contacting the first nucleic acid, the second nucleic acid, the third nucleic acid, and the fourth nucleic acid; and amplifying the resulting product. Further provided herein are methods further comprising an error correction step. Further provided herein are methods further comprising contacting a reaction mixture comprising an exonuclease, an endonuclease, a polymerase, and a ligase during step of contacting the first nucleic acid, the second nucleic acid, the third nucleic acid, and the fourth nucleic acid. Further provided herein are methods, wherein the first hypervariable region sequence and the second hypervariable region sequence each comprises a complementary Further provided herein are methods, wherein the first nucleic acid comprises about 300 to about 700 base pairs. Further provided herein are methods, wherein the second nucleic acid comprises about 200 to about 600 base pairs. Further provided herein are methods, wherein the third nucleic acid comprises about 200 to about 600 base pairs. Further provided herein are methods, wherein the fourth nucleic acid comprises about 200 to about 600 base pairs.

Provided herein are methods for nucleic acid assembly, comprising: de novo synthesizing: a first nucleic acid comprising in 5′ to 3′ order: a first complementary region adjacent to a first variable region sequence and a first variable region sequence; a second nucleic acid comprising in 5′ to 3′ order: a first region of fixed variability sequence and a first hypervariable region sequence; a third nucleic acid comprising a second variable region sequence; a fourth nucleic acid comprising in 5′ to 3′ order: a restriction endonuclease site and a second region of fixed variability sequence; and a fifth nucleic acid comprising in 5′ to 3′ order: the second region of fixed variability sequence, a second hypervariable region sequence, and a variable constant region sequence; contacting the first nucleic acid, the second nucleic acid, the third nucleic acid, the fourth nucleic acid, and the fifth nucleic acid with a reaction mixture comprising an exonuclease, an endonuclease, a polymerase, and a ligase; and cloning a construct of step (b) into a vector sequence. Further provided herein are methods, wherein the first hypervariable region sequence and the second hypervariable region sequence each comprises a complementary determining region (CDR). Further provided herein are methods, wherein the CDR is CDR3. Further provided herein are methods further comprising contacting one or more variable constant regions. Further provided herein are methods, wherein the exonuclease is exonuclease III. Further provided herein are methods, wherein the flap endonuclease is flap endonuclease 1, exonuclease 1, XPG, Dna2, or GEN1. Further provided herein are methods, wherein the polymerase comprises 5′ to 3′ polymerase activity.

Provided herein are methods for nucleic acid assembly, comprising: providing a first nucleic acid comprising in 5′ to 3′ order: a first complementary region adjacent to a first variable region sequence and a first variable region sequence; providing a second nucleic acid sequence comprising in 5′ to 3′ order: a first region of fixed variability sequence, a first hypervariable region sequence, a restriction endonuclease site, a second hypervariable region sequence, and a universal primer; amplifying the first nucleic acid and the second nucleic acid to generate a third nucleic acid; providing a vector sequence comprising the first complementary region adjacent to the first variable region sequence and a first region of any defined length sequence; contacting the third nucleic acid and the vector sequence; contacting a fourth nucleic acid comprising in 5′ to 3′ order: a self-cleaving peptide sequence, a second complementary region adjacent to a second variable region sequence, and a second variable region sequence. Further provided herein are methods, wherein the first hypervariable region sequence and the second hypervariable region sequence each comprises a complementary determining region (CDR). Further provided herein are methods, wherein the CDR is CDR3. Further provided herein are methods, wherein the self-cleaving peptide is P2A.

Provided herein are methods for nucleic acid assembly, comprising: de novo synthesizing: a first nucleic acid comprising a first complementary region adjacent to a first variable region sequence and a first variable region sequence; a second nucleic acid comprising a first hypervariable region sequence; a third nucleic acid comprising a second variable region sequence; a fourth nucleic acid comprising in 5′ to 3′ order: a first hypervariable region sequence, a first region of fixed variability, and a barcode; amplifying the first nucleic acid and the second nucleic acid to generate a fifth nucleic acid; amplifying the third nucleic acid and the fourth nucleic acid to generate a fifth nucleic acid; contacting the fifth nucleic acid and the sixth nucleic acid with a reaction mixture comprising an exonuclease, an endonuclease, a polymerase, and a ligase to generate a seventh nucleic acid; circularizing the seventh nucleic acid; sequencing and identifying the seventh nucleic acid using the barcode; amplifying the seventh nucleic acid; and assembling the seventh nucleic acid in a vector using the reaction mixture comprising the exonuclease, the endonuclease, the polymerase, and the ligase. Further provided herein are methods, wherein the first variable region sequence or the second variable region sequence is amplified with a hypervariable region sequence. Further provided herein are methods, wherein the hypervariable region sequence comprises a CDR. Further provided herein are methods, wherein the CDR is CDR3. Further provided herein are methods further comprising contacting with sequences comprising one or more regions of any defined length. Further provided herein are methods, wherein about 100 variants of the first variable region sequence are synthesized. Further provided herein are methods, wherein about 130 variants of the second variable region sequence are synthesized. Further provided herein are methods, wherein the self-cleaving peptide is P2A. Further provided herein are methods, wherein the exonuclease is exonuclease III. Further provided herein are methods, wherein the endonuclease is a flap endonuclease. Further provided herein are methods, wherein the flap endonuclease is flap endonuclease 1, exonuclease 1, XPG, Dna2, or GEN1. Further provided herein are methods, wherein the polymerase comprises 5′ to 3′ polymerase activity. Further provided herein are methods, wherein the polymerase is a DNA polymerase. Further provided herein are methods, wherein the ligase catalyzes joining of at least two nucleic acids. Further provided herein are methods, wherein the first region of fixed variability sequence and the second region of fixed variability sequence are each about 10 to about 100 base pairs. Further provided herein are methods, wherein the first region of fixed variability sequence and the second region of fixed variability sequence are each about 40 base pairs.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A illustrates a schematic of a combinatorial assembly with modular inputs (2 inputs or “domains” shown for illustration only) and pools connected by a unique linker region.

FIG. 1B illustrates a schematic of paired variant assembly using a Type IIS exposed barcode.

FIG. 2 illustrates a schematic of paired variant assembly using paired homology.

FIG. 3 illustrates a schematic of de novo synthesis of variant nucleic acids, such as those encoding for immunoglobulins or fragments thereof.

FIG. 4 illustrates a schematic of paired variant assembly using paired homology into a vector.

FIG. 5A illustrates a schematic paired variant assembly using Type IIS into a vector.

FIG. 5B illustrates a schematic of nucleic acid assembly using paired barcodes and dial out PCR.

FIG. 6 illustrates a schematic of nucleic acid assembly using polynucleotide populations specific for each variable region.

FIG. 7 depicts systems for polynucleotide synthesis and nucleic acid assembly.

FIG. 8 illustrates a computer system.

FIG. 9 is a block diagram illustrating architecture of a computer system.

FIG. 10 is a block diagram of a multiprocessor computer system using a shared virtual address memory space.

FIG. 11 is a diagram demonstrating a network configured to incorporate a plurality of computer systems, a plurality of cell phones and personal data assistants, and Network Attached Storage (NAS).

FIG. 12A is a graph of colony forming units (CFUs).

FIG. 12B is a graph of colony forming units (CFUs) of A/T rich overlap homology sequences.

FIG. 12C is a graph of pass rates of Comparator 1 and Comparator 2.

FIG. 12D is a graph of assembly specificity and sequence bias by an enzymatic assembly method by percent of the population comprising three assembled genes. Assembly of three different genes (Gene A, Gene B, Gene C), composed of 9 dsDNA input fragments with adapters were assembled in a single reaction.

FIG. 12E is a graph of colony forming units (CFUs) for assembly of zero to six DNA fragments at once using an enzymatic assembly method.

FIG. 12F is a graph of colony forming units (CFUs) for assembly of zero to ten DNA fragments at once using an enzymatic assembly method, Comparator 1, or Comparator 2.

FIG. 12G is a graph of colony forming units (CFUs) for either 25 bp or 40 bp overlap homology regions using an enzymatic assembly method.

FIG. 13A shows relative concentrations of DNA following PCR using universal primers following multiplex assembly.

FIG. 13B shows a plot from a BioAnalyzer reading following multiplex assembly.

FIG. 13C shows a density plot using 140× coverage of populations of genes following multiplex assembly.

FIG. 13D shows percentage of insertion/deletion free in populations of genes following multiplex assembly of a 400 bp gene pool.

FIG. 13E shows percentage of insertion/deletion free in populations of genes following multiplex assembly.

FIG. 13F shows percentage of complete dropout, dropout, and runaway in populations of genes following multiplex assembly.

FIG. 13G shows a graph of soft clipping/chimeric reads in populations of genes following multiplex assembly.

FIG. 14A is a graph of uniformity of full length sequences before and after cloning of combinatorial assembly using four populations of gene fragments.

FIG. 14B is a graph of frequency of variants within a domain following combinatorial assembly using four populations of gene fragments.

FIG. 15A are graphs of frequency density vs. log(read counts) for a pre-cloned pool (left) and cloned pool (right).

FIG. 15B is a graph of frequency of variants within a domain following combinatorial assembly using four populations of gene fragments.

FIG. 15C are graphs of frequency density vs. log(read counts) for a 4×4 assembly (left) and 10×10 assembly (right).

FIG. 15D are graphs of frequency density vs. log(read counts) for a 50×50 combinatorial assembly (left) and 100×100 combinatorial assembly (right).

FIG. 16A is a graph of sequence diversity (base counts) as a function of position in the gene pool for a 250 k sequence combinatorial library encoding for viral proteins.

FIG. 16B is a graph of sequence representation across sequences with varying GC content for a 250 k sequence combinatorial library encoding for viral proteins.

FIG. 16C is a graph of the size distribution of genes in a 250 k sequence combinatorial library encoding for viral proteins.

FIG. 16D is a graph of frequency density vs. log(read counts) for a 250 k sequence combinatorial library encoding for viral proteins.

FIG. 16E is a graph of uniformity across 11 sub gene pools.

FIG. 16F are graphs of pool characteristics, including: drop outs (missing from pool), under represented (<10× of the mean), runaway (>10× of the mean), and percent genes with perfect sequences.

### DETAILED DESCRIPTION

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “nucleic acid” encompasses double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands). Nucleic acid sequences, when provided, are listed in the 5′ to 3′ direction, unless stated otherwise. Methods described herein provide for the generation of isolated nucleic acids. Methods described herein additionally provide for the generation of isolated and purified nucleic acids. A “nucleic acid” as referred to herein can comprise at least 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, 2000, or more bases in length. Moreover, provided herein are methods for the synthesis of any number of polypeptide-segments encoding nucleotide sequences, including sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g. promoters, transcription factors, enhancers, siRNA, shRNA, RNAi, miRNA, small nucleolar RNA derived from microRNA, or any functional or structural DNA or RNA unit of interest. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. cDNA encoding for a gene or gene fragment referred to herein may comprise at least one region encoding for exon sequences without an intervening intron sequence in the genomic equivalent sequence.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

Primers referred to in the exemplary workflows mentioned herein as “universal primers,” are short polynucleotides that recognize a primer binding site common to multiple DNA fragments. However, these workflows are not limited to only use of universal primers, and fragment-specific primers may be incorporated in addition or alternatively. In addition, while exemplary workflows described herein refer to assembly of gene fragments, they are not limited as such and are applicable to the assembly of longer nucleic acids in general.

Sequence Assembly

Described herein are methods and compositions for the assembly of nucleic acid sequences. Assembly of such sequences may in some cases be challenging due to specific properties of the assembly fragments, such as GC content, repeating regions, and secondary structure. Additionally, assembly of libraries of such sequences may be assembled in parallel, with members of the library possessing regions of high variability across members. Such parallel assembly of fragments is challenging due to the presence of highly variable regions across members of the library for such fragments. Moreover, assembly may result in errors, such as incorrectly assembled nucleic acids. Nucleic acids comprising variable regions may include nucleic acids encoding for genes (such as proteins or antibodies), or non-coding nucleic acids. In some instances, a nucleic acid assembled herein comprises a region encoding for an immunoglobulin or fragment thereof. Assembly of libraries comprising nucleic acids of high variability may be accomplished by the methods described herein. Such methods in some instances comprise PCR/PCA-based overlap assembly, ligation, cloning with vectors, flapase-based assembly, exonuclease-based assembly, or other assembly method. Multiple methods are in some instances combined to generate a library of nucleic acids. Such methods are executed in any order, and in some instances comprise intervening purification or other steps. In some instances, assembled nucleic acids are amplified from a pool of partially and fully assembled nucleic acids to generate a library. In some instances, correctly assembled nucleic acids are amplified from a pool comprising correctly assembled and incorrectly assembled nucleic acids to generate a library.

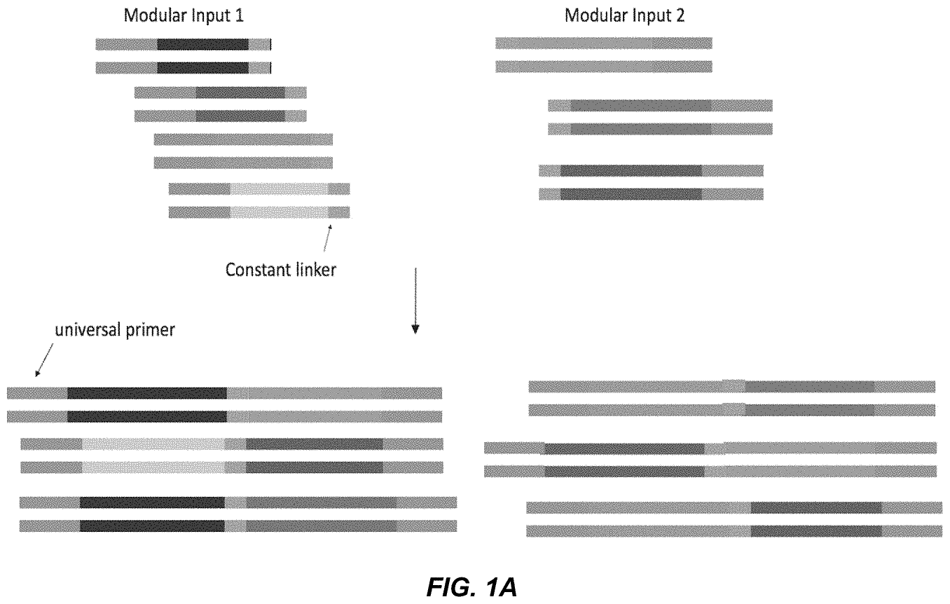

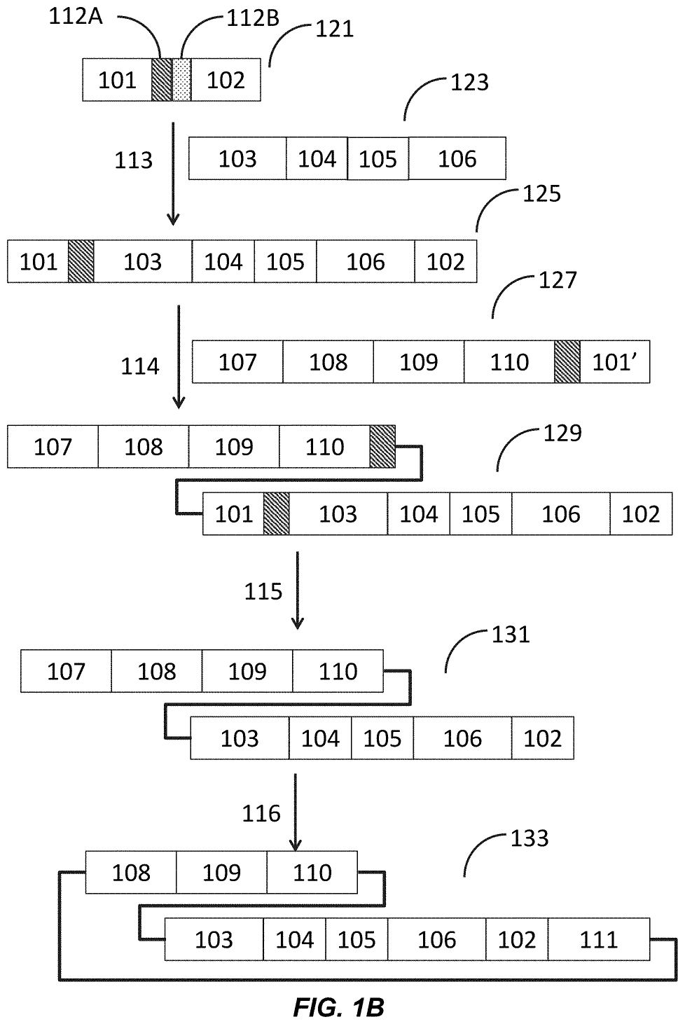

An exemplary process for sequence assembly using a barcode is seen in FIG. 1B. Gene fragment 121 is synthesized and comprises a barcode 101 followed by a first restriction endonuclease site 112A, a second restriction endonuclease site 112B, and a first hypervariable region 102. In some instances, the first hypervariable region comprises a CDR. In some instances, the CDR is CDR3. In some instances, the first restriction endonuclease site or the second restriction endonuclease site is a Type IIS restriction endonuclease (TIIS-RE) site. In some instances, the first restriction endonuclease site and the second restriction endonuclease site are different TIIS-RE sites. Gene fragment 123 is synthesized and comprises a first region of any defined length 103 followed by a self-cleaving peptide sequence 104, a first complementary region adjacent to a first variable region 105, and a first variable region 106. In some instances, the self-cleaving peptide sequence is P2A. In some instances, the number of first variable regions synthesized is about 100. In some instances, the number of first variable regions synthesized is about 50, 100, 150, 200, 250, 300, 500, 1000, or about 2000. In some instances, the number of first variable regions synthesized is 10-100, 20-1000, 50-1000, 100-1000, 25-500, 75-125, 200-2000, 150-2000, 300-5000, 50-5000, 1000-5000, or 50-300. Gene fragment 121 is combined 113 with gene fragment 123. The resulting fragment 125 comprises the barcode 101 followed by the restriction endonuclease site 112A, the first region of any defined length 103, the cleaving peptide sequence 104, the first complementary region adjacent to a first variable region 105, the first variable region 106, and the first hypervariable region 102. Gene fragment 127 is synthesized and comprises a vector sequence 107 followed by a second complementary region adjacent to a second variable region 108, a second variable region 109, a second hypervariable region 110, a TIIS-RE site 112A, and a second barcode 101′. In some instances, the number of second variable regions synthesized is about 130. In some instances, the number of second variable regions synthesized is about 50, 100, 150, 200, 250, 300, 500, 1000, or about 2000. In some instances, the number of first variable regions synthesized is 10-100, 20-1000, 50-1000, 100-1000, 25-500, 75-125, 200-2000, 150-2000, 300-5000, 50-5000, 1000-5000, or 50-300. Gene fragment 125 is then PCR amplified 114 with gene fragment 127. The resulting fragment 129 comprises the vector sequence 107 followed by the second complementary region adjacent to a second variable region 108, the second variable region 109, the second hypervariable region 110, the TIIS-RE site 112A, the barcode 101, the TIIS-RE site 112A, the first region of any defined length 103, the cleaving peptide sequence 104, the first complementary region adjacent to a first variable region 105, the first variable region 106, and the first hypervariable region 102. Gene fragment 129 is then cloned and the TIIS restriction endonucleases cut at the TIIS-RE sites to remove the barcode 101. The resulting fragment 131 comprises the vector sequence 107 followed by the second complementary region adjacent to a second variable region 108, the second variable region 109, the second hypervariable region 110, the first region of any defined length 103, the cleaving peptide sequence 104, the first complementary region adjacent to a first variable region 105, the first variable region 106, and the first hypervariable region 102. Gene fragment 131 is then cloned 116 to generate final construct 133. The final construct 133 comprises the second complementary region adjacent to a second variable region 108, the second variable region 109, the second hypervariable region 110, the first region of any defined length 103, the cleaving peptide sequence 104, the first complementary region adjacent to a first variable region 105, the first variable region 106, the first hypervariable region 102, and a first variable constant segment 111. In some instances, a number of final constructs generated is about 1000. In some instances, the number of gene fragments synthesized is about 50, 100, 250, 500, 1000, 2000, 3000, 5000, 7500, 10,000, or about 20,000. In some instances, the number of first variable regions synthesized is 100-5000, 200-5000, 500-5000, 100-2000, 250-1500, 750-1250, 2000-7500, 900-10,000, 3000-10,000, 750-5000, 500-2000, or 500-3000. In some instances, the number of final constructs synthesized is about 5000, 10,000, 25,000, 500,000, 100,000, 200,000, 300,000, 500,000, 750,000, 1,000,000, or about 5,000,000. In some instances, the number of final constructs synthesized is at least 5000, 10,000, 25,000, 500,000, 100,000, 200,000, 300,000, 500,000, 750,000, 1,000,000, or at least 5,000,000.

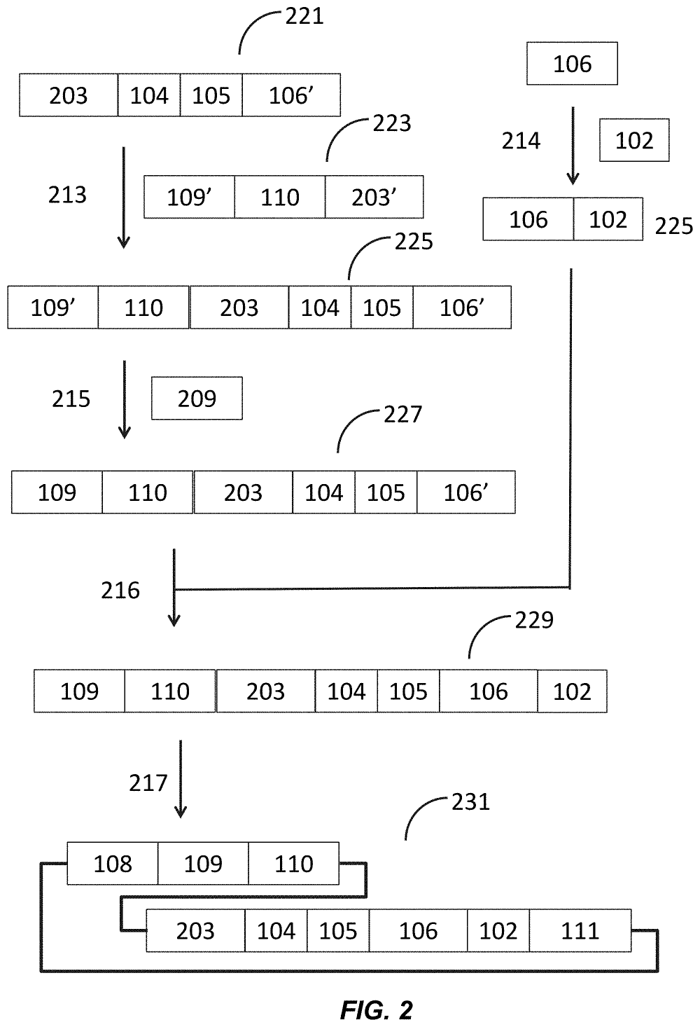

An exemplary process for sequence assembly is seen in FIG. 2. Gene fragment 221 is synthesized and comprises a second region of any defined length 203, a self-cleaving peptide sequence 104, a first complementary region adjacent to a first variable region 105, and a first region of fixed variability 106′. In some instances, the first region of fixed variability is at least 15, 20, 25, 30, 35, 40, 45, 50, or more than 50 bases in length. In some instances, the first region of fixed variability is about 15, 20, 25, 30, 35, 40, 45, 50, 55, 60 or about 65 bases in length. In some instances, the self-cleaving peptide sequence is P2A. Gene fragment 223 is synthesized and comprises a second region of fixed variability 109′ followed by the second hypervariable region 110 and a region 203′ that is homologous to the second region of any defined length 203. In some instances, the second hypervariable region comprises a CDR. In some instances, the CDR is CDR3. Gene fragment 221 is PCR amplified 213 with gene fragment 223 to generate gene fragment 225. Gene fragment 225 comprises segment 109′, the second hypervariable region 110, the second region of any defined length 203, the self-cleaving peptide sequence 104, the first complementary region adjacent to a first variable region 105, and the first region of fixed variability 106′. Gene fragment 225 and gene fragment 209 are subject to enzymatic based assembly and PCR amplified 215 to generate gene fragment 227. Gene fragment 227 comprises the second variable region 109 followed by the second hypervariable region 110, the second region of any defined length 203, the self-cleaving peptide sequence 104, the first complementary region adjacent to a first variable region 105, and the first region of fixed variability 106′. In a separate reaction, a first variable region 106 is synthesized homologous to the first region of fixed variability 106′. The first variable region 106 is amplified 214 with the first hypervariable region 102 to generate gene fragment 225 comprising the first variable region 106 followed by the first hypervariable region 102. Gene fragment 225 and gene fragment 227 are then combined and subject to enzymatic based assembly 216 to generate gene fragment 229. Gene fragment 229 comprises the second variable region 109 followed by the second hypervariable region 110, the second region of any defined length 203, the self-cleaving peptide sequence 104, the first complementary region adjacent to a first variable region 105, the first variable region 106, and the first hypervariable region 102. Gene fragment 229 is cloned 217 into a vector to generate final construct 231. Construct 231 comprises the second complementary region adjacent to a second variable region 108 followed by the second variable region 109, the second hypervariable region 110, the second region of any defined length 203, the self-cleaving peptide sequence 104, the first complementary region adjacent to a first variable region 105, the first variable region 106, the first hypervariable region 102, and the first variable constant segment 111. In some instances, the number of first variable regions synthesized is about 50, 100, 150, 200, 250, 300, 500, 1000, or about 2000. In some instances, the number of first variable regions synthesized is 10-100, 20-1000, 50-1000, 100-1000, 25-500, 75-125, 200-2000, 150-2000, 300-5000, 50-5000, 1000-5000, or 50-300. In some instances, the number of second variable regions synthesized is about 50, 100, 150, 200, 250, 300, 500, 1000, or about 2000. In some instances, the number of first variable regions synthesized is 10-100, 20-1000, 50-1000, 100-1000, 25-500, 75-125, 200-2000, 150-2000, 300-5000, 50-5000, 1000-5000, or 50-300. In some instances, the number of gene fragments synthesized is about 50, 100, 250, 500, 1000, 2000, 3000, 5000, 7500, 10,000, or about 20,000. In some instances, the number of first variable regions synthesized is 100-5000, 200-5000, 500-5000, 100-2000, 250-1500, 750-1250, 2000-7500, 900-10,000, 3000-10,000, 750-5000, 500-2000, or 500-3000.

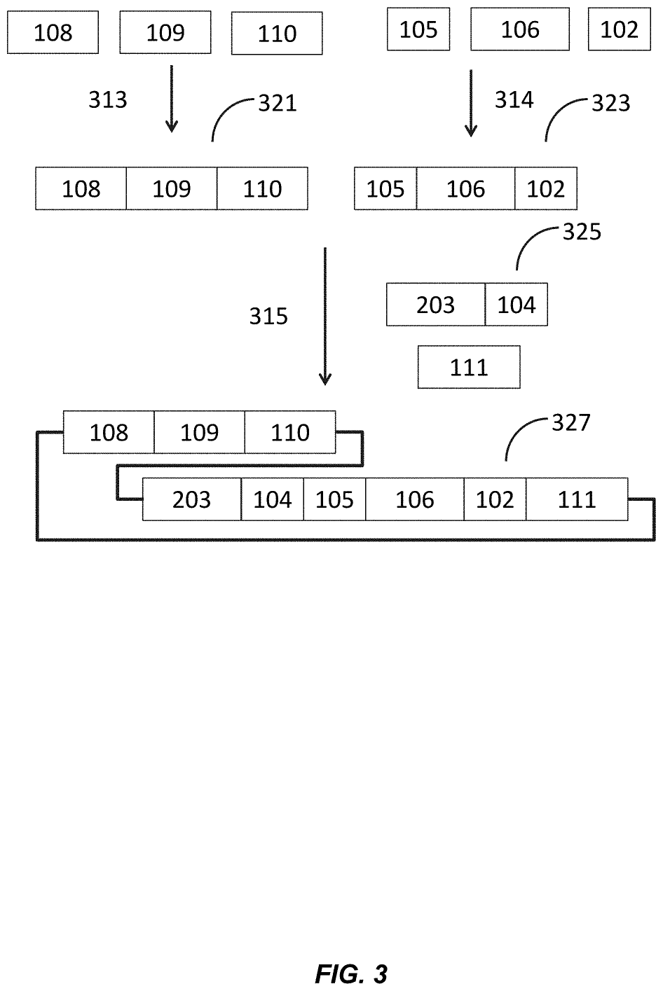

An exemplary de novo synthesis method is seen in FIG. 3. A first complementary region adjacent to a first variable region 105, a first variable region 106, and a first hypervariable region 102 are synthesized and then subject to polymerase cycling assembly (PCA) 314 to generate gene fragment 323. Gene fragment 323 comprises the first complementary region adjacent to a first variable region 105 followed by the first variable region 106 and the first hypervariable region 102. In some instances, the first hypervariable region comprises a CDR. In some instances, the CDR is CDR3. A second complementary region adjacent to a second variable region 108, a second variable region 109, and a second hypervariable region 110 are synthesized and subject to assembly PCR or PCA 313 to generate gene fragment 321. In some instances, the second hypervariable region comprises a CDR. In some instances, the CDR is CDR3. Gene fragment 321 comprises the second complementary region adjacent to a second variable region 108 followed by the second variable region 109 and the second hypervariable region 110. Clones of gene fragment 325 comprising a second region of any defined length 203 followed by a self-cleaving peptide sequence 104 and the first variable constant segment 111 are synthesized. Each gene fragment 321, 323, and 325 are synthesized in individual wells and PCR amplified. Gene fragment 325 and the first variable constant segment 111 are added to gene fragment 321 and gene fragment 323 to generate gene fragment 327 followed by PCR. In some instances, an error correction reaction is performed. Gene fragment 327 comprises the second complementary region adjacent to a second variable region 108 followed by the second variable region 109, the second hypervariable region 110, the second region of any defined length 203, the self-cleaving peptide sequence 104, the first complementary region adjacent to a first variable region 105, the first variable region 106, the first hypervariable region 102, and the first variable constant segment 111. Gene fragment 327 is then cloned and subject to next generation sequencing. In some instances, the number of first variable regions synthesized is about 50, 100, 150, 200, 250, 300, 500, 1000, or about 2000. In some instances, the number of first variable regions synthesized is 10-100, 20-1000, 50-1000, 100-1000, 25-500, 75-125, 200-2000, 150-2000, 300-5000, 50-5000, 1000-5000, or 50-300. In some instances, the number of second variable regions synthesized is about 50, 100, 150, 200, 250, 300, 500, 1000, or about 2000. In some instances, the number of first variable regions synthesized is 10-100, 20-1000, 50-1000, 100-1000, 25-500, 75-125, 200-2000, 150-2000, 300-5000, 50-5000, 1000-5000, or 50-300. In some instances, the number of gene fragments synthesized is about 50, 100, 250, 500, 1000, 2000, 3000, 5000, 7500, 10,000, or about 20,000. In some instances, the number of first variable regions synthesized is 100-5000, 200-5000, 500-5000, 100-2000, 250-1500, 750-1250, 2000-7500, 900-10,000, 3000-10,000, 750-5000, 500-2000, or 500-3000.

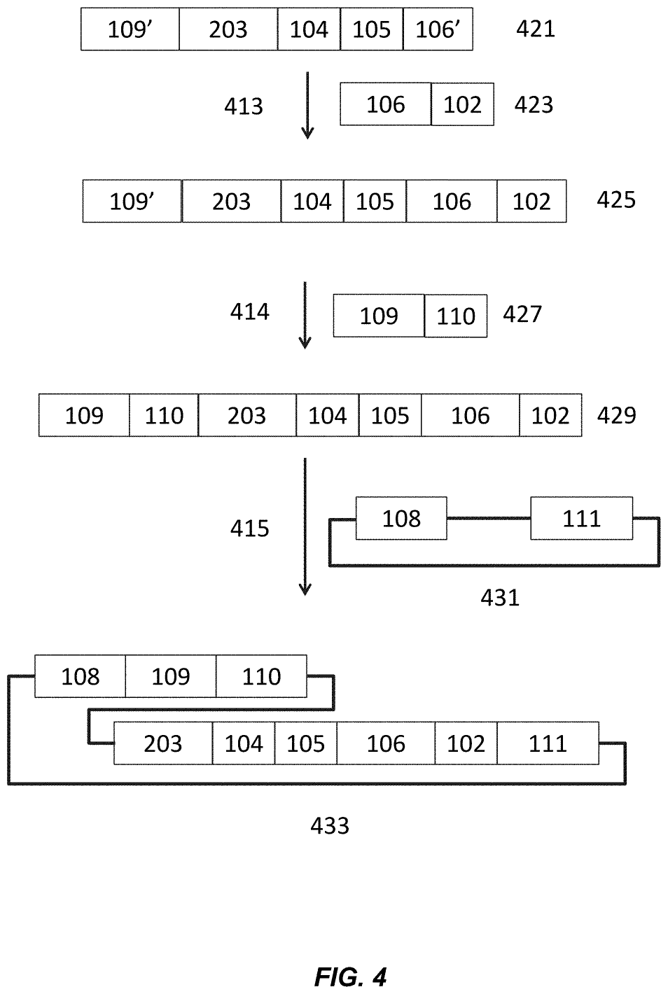

Provided herein are methods for paired variant assembly using paired homology. An exemplary process is seen in FIG. 4. Gene fragment 421 is synthesized comprising a second region of fixed variability 109′ followed by the second region of any defined length 203, the self-cleaving peptide sequence 104, the first complementary region adjacent to a first variable region 105, and the first region of fixed variability 106′. In some instances, the base pair region complementary to the second variable region is at least 15, 20, 25, 30, 35, 40, 45, 50, or more than 50 bases in length. In some instances, the base pair region complementary to the second variable region is about 15, 20, 25, 30, 35, 40, 45, 50, 55, 60 or about 65 bases in length. In some instances, about 130 variants comprising the sequence homologous to the first hypervariable region, the second region of any defined length, the self-cleaving peptide sequence, the first complementary region adjacent to a first variable region, and the region of fixed variability are synthesized. In some instances, the first hypervariable region comprises a CDR. In some instances, the CDR is CDR3. Gene fragment 421 is combined 413 with gene fragment 423 that comprises the first variable region 106 and the first hypervariable region 102 to generate gene fragment 425. In some instances, about 100 variants comprising the first variable segment and the first hypervariable region are synthesized. Gene fragment 425 comprises the second region of fixed variability 109′ followed by the second region of any defined length 203, a self-cleaving peptide sequence 104, the first complementary region adjacent to a first variable region 105, the first variable region 106, and the first hypervariable region 102. Gene fragment 425 is then combined 414 with gene fragment 427 comprising the second variable region 109 and the second hypervariable region 110 to generate gene fragment 429. In some instances, about 130 variants comprising the second variable region and the second hypervariable region are synthesized. In some instances, the second hypervariable region comprises a CDR. In some instances, the CDR is CDR3. Gene fragment 429 comprises the second variable region 109, the second hypervariable region 110, the second region of any defined length 203, a self-cleaving peptide sequence 104, the first complementary region adjacent to a first variable region 105, the first variable region 106, and the first hypervariable region 102. Gene fragment 429 is then pooled and cloned 415 into a destination vector 431. The destination vector 431 comprises the second complementary region adjacent to a second variable region 108 and the first variable constant segment 111. The resulting construct 433 comprises the second complementary region adjacent to a second variable region 108 followed by the second variable region 109, the second hypervariable region 110, the second region of any defined length 203, a self-cleaving peptide sequence 104, the first complementary region adjacent to a first variable region 105, the first variable region 106, and the first hypervariable region 102, and the first variable constant segment 111.

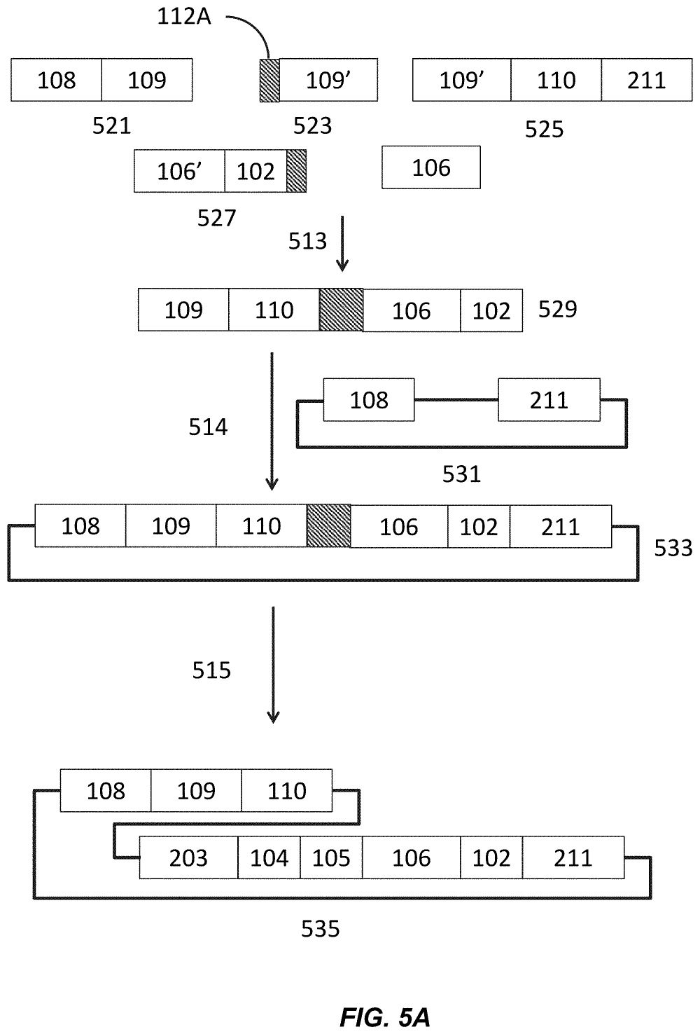

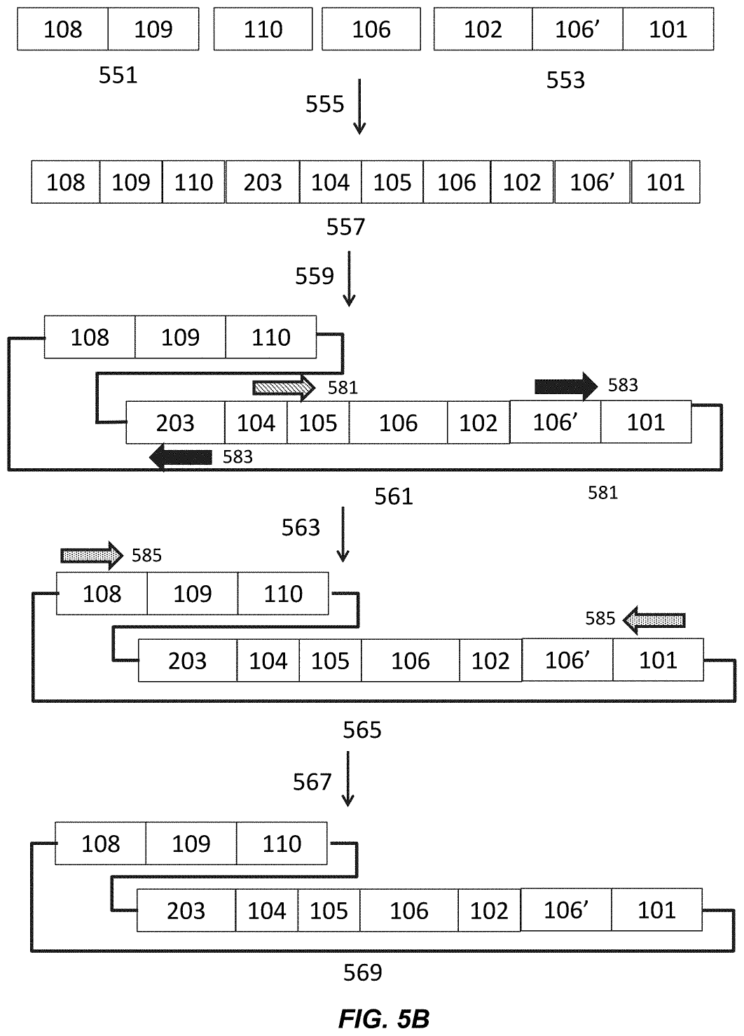

An exemplary process for sequence assembly is seen in FIG. 5A. Gene fragment 521 is synthesized and comprises the second complementary region adjacent to a second variable region 108 and the second variable region 109. Gene fragment 523 is synthesized and comprises the first restriction endonuclease site 112A followed by the second region of fixed variability 109′. In some instances, second region of fixed variability is at least 15, 20, 25, 30, 35, 40, 45, 50, or more than 50 bases in length. In some instances, the second region of fixed variability is about 15, 20, 25, 30, 35, 40, 45, 50, 55, 60 or about 65 bases in length. In some instances, second region of fixed variability is 10-60, 10-40, 15-60, 20-60, 20-80, 30-50, 20-45, 35-55, 40-80, or 50-80. Gene fragment 525 is synthesized and comprises the second region of fixed variability 109′ followed by the second hypervariable region 110 and a second variable constant segment 211. Gene fragment 527 is synthesized and comprises the first region of fixed variability 106′ followed by the first hypervariable region 102 and the first restriction endonuclease site 112A. In some instances, the first hypervariable region comprises a CDR. In some instances, the second hypervariable region comprises a CDR. In some instances, the CDR is CDR3. In some instances, the restriction endonuclease site is a TIIS-RE site. Gene fragments 521, 523, 525, 527, and the first variable region 106 are pooled and PCR amplified 513 in order to add the first hypervariable region 102 and the second hypervariable region 110. The resulting gene fragment 529 comprises the second variable region 109 followed by the second hypervariable region 110, the first restriction endonuclease site 112A, the first variable region 106, and the first hypervariable region 102. Gene fragment 529 and destination vector 531 comprising the second complementary region adjacent to a second variable region 108 and the second variable constant segment 211 are then subjected to flap endonuclease mediated nucleic acid assembly 514 to generate gene fragment 533. Gene fragment 533 comprises the second complementary region adjacent to a second variable region 108 followed by the second variable region 109, the second hypervariable region 110, the first restriction endonuclease site 112A, the first variable region 106, the first hypervariable region 102, and the second variable constant segment 211. Gene fragment 533 is then subjected to Golden Gate Assembly 515 to insert the second region of any defined length 203 to generate final construct 535. Final construct 535 comprises the second complementary region adjacent to a second variable region 108 followed by the second variable region 109, the second hypervariable region 110, the second region of any defined length 203, the self-cleaving peptide sequence 104, the first complementary region adjacent to a first variable region 105, the first variable region 106, the first hypervariable region 102, and the second variable constant segment 211. A number of final constructs generated, in some instances, is about 10000. In some instances, the number of first variable regions synthesized is about 50, 100, 150, 200, 250, 300, 500, 1000, or about 2000. In some instances, the number of first variable regions synthesized is 10-100, 20-1000, 50-1000, 100-1000, 25-500, 75-125, 200-2000, 150-2000, 300-5000, 50-5000, 1000-5000, or 50-300. In some instances, the number of second variable regions synthesized is about 50, 100, 150, 200, 250, 300, 500, 1000, or about 2000. In some instances, the number of first variable regions synthesized is 10-100, 20-1000, 50-1000, 100-1000, 25-500, 75-125, 200-2000, 150-2000, 300-5000, 50-5000, 1000-5000, or 50-300. In some instances, the number of final constructs synthesized is about 5000, 10,000, 25,000, 500,000, 100,000, 200,000, 300,000, 500,000, 750,000, 1,000,000, or about 5,000,000. In some instances, the number of final constructs synthesized is at least 5000, 10,000, 25,000, 500,000, 100,000, 200,000, 300,000, 500,000, 750,000, 1,000,000, or at least 5,000,000. In some instances, the number of first variable regions synthesized is 1000-50,000, 2900-50,000, 5000-50,000, 1000-20,000, 2500-15,000, 7500-12,500, 20,000-75,000, 9000-100,000, 30,000-100,000, 7500-50,000, 5000-20,000, or 5000-30,000.

An exemplary process for sequence assembly is seen in FIG. 5B. Gene fragment 551 is synthesized and comprises the second complementary region adjacent to a second variable region 108 and the second variable region 109. Gene fragment comprising the second hypervariable region 110 is synthesized. Gene fragment comprising the first variable region 106 is synthesized. Gene fragment 553 comprising a first hypervariable region 102 followed by the first region of fixed variability 106′ and the barcode 101. A first combinatorial library of gene fragment 551 and the second hypervariable region 110 are generated using PCR. A second combinatorial library of gene fragment 553 and the first variable region 106 are generated using PCR. The first combinatorial library and the second combinatorial library are assembled using enzymatic based assembly 555 to generate fragment 559. Gene fragment 557 comprises the second complementary region adjacent to a second variable region 108 followed by the second variable region 109, the second hypervariable region 110, the second region of any defined length 203, the self-cleaving peptide sequence 104, the first complementary region adjacent to a first variable region 105, the first variable region 106, the first hypervariable region 102, the first region of fixed variability 106′, and the barcode 101. In some instances, gene fragment 559 comprises a region of a fixed number of base pairs. The number of base pairs, in some instances, is at least or about 5, 10, 15, 20, 25, 30, 35, 40, 45, 50, or more than 50 base pairs. Gene fragment 557 is circularized 559 to generate gene fragment 561. Gene fragment 561 comprises the second complementary region adjacent to a second variable region 108 followed by the second variable region 109, the second hypervariable region 110, the second region of any defined length 203, the self-cleaving peptide sequence 104, the first complementary region adjacent to a first variable region 105, the first variable region 106, the first hypervariable region 102, the first region of fixed variability 106′, and the barcode 101. The first variable region and the first hypervariable region may comprise varying lengths. In some instances, the length of the first variable region and the first hypervariable region is at least or about 15, 20, 25, 30, 35, 40, 45, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, or more than 700 bases in length. In some instances, the length of the first variable region and the first hypervariable region is in a range of about 10-1000, 50-900, 100-800, or 200-600 base pairs. The second variable region and the second hypervariable region may comprise varying lengths. In some instances, the length of the second variable region and the second hypervariable region is at least or about 15, 20, 25, 30, 35, 40, 45, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, or more than 700 bases in length. In some instances, the length of the second variable region and the second hypervariable region is in a range of about 10-1000, 50-900, 100-800, or 200-600 base pairs. In some instances, the second region of any defined length, the self-cleaving peptide sequence, and the first complementary region adjacent to a first variable region comprise varying lengths. In some instances, the length of the second region of any defined length, the self-cleaving peptide sequence, and the first complementary region adjacent to a first variable region is at least or about 15, 20, 25, 30, 35, 40, 45, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 1000, or more than 1000 bases in length. In some instances, the length of the second region of any defined length, the self-cleaving peptide sequence, and the first complementary region adjacent to a first variable region is in a range of about 10-1000, 50-900, 100-800, or 200-600 base pairs. In some instances, the first region of fixed variability comprises at least or about 15, 20, 25, 30, 35, 40, 45, 50, 60, 70, 80, 90, 100, or more than 100 base pairs. In some instances, the barcode comprises at least or about 1, 5, 10, 15, 20, 25, 30, 35, 40, 45, 50, or more than 50 base pairs. Gene fragment 561 is then sequenced with primers 581 and 583 and samples are identified having the barcode 563 to generate gene fragment 565. Gene fragment 565 is then subject to dial out PCR and enzymatic based assembly 567 into a final vector 569.

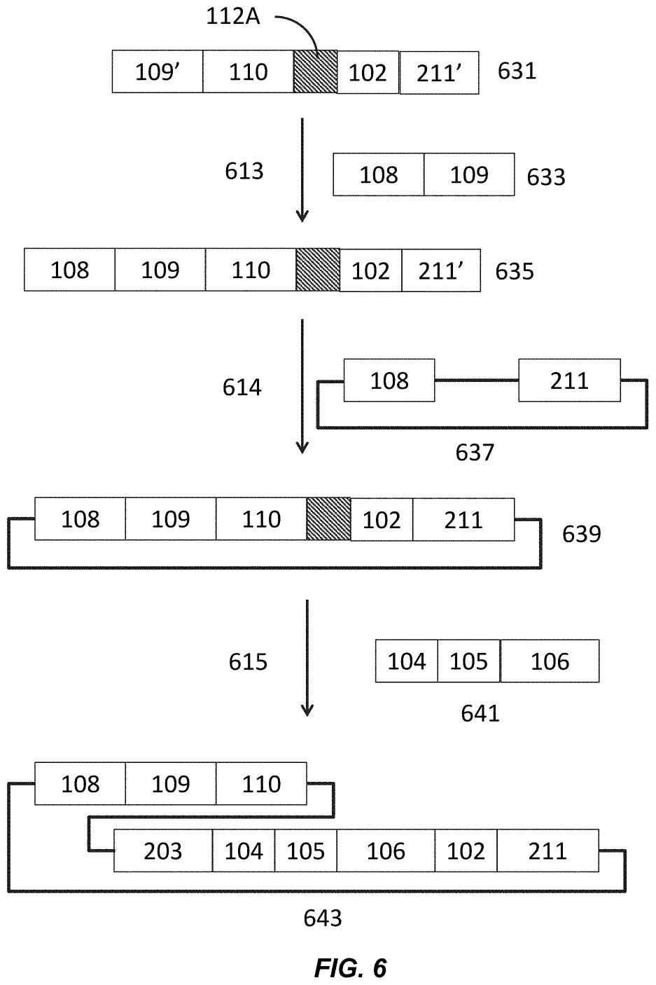

An exemplary process using populations specific for each variant is seen in FIG. 6. Gene fragment 631 is synthesized comprising the second region of fixed variability 109′ followed by the second hypervariable region 110, the first restriction endonuclease site 112A, the first hypervariable region 102, and universal primer 211′. In some instances, the first hypervariable region comprises a CDR. In some instances, the second hypervariable region comprises a CDR. In some instances, the CDR is CDR3. In some instances, the restriction endonuclease site is a TIIS-RE site. Gene fragment 631 is combined and PCR amplified 613 with a population of gene fragments 633 comprising the second complementary region adjacent to a second variable region 108 followed by the second variable region 109 to generate gene fragment 635. Gene fragment 635 comprises the second complementary region adjacent to a second variable region 108 followed by the second variable region 109, the second hypervariable region 110, the first restriction endonuclease site 112A, the first hypervariable region 106, and universal primer 211′. Gene fragment 635 is then assembled 614 into a destination vector 637 comprising the second complementary region adjacent to a second variable region 108 and the second variable constant segment 211 to generate gene fragment 639. Gene fragment 639 comprises the second complementary region adjacent to a second variable region 108 followed by the second variable region 109, the second hypervariable region 110, the first restriction endonuclease site 112A, the first hypervariable region 102, and the second variable constant segment 211. Gene fragment 641 is synthesized and comprises the self-cleaving peptide sequence 104, the first complementary region adjacent to a first variable region 105, and the first variable region 106. Gene fragment 639 and gene fragment 641 are assembled 615 to insert the second region of any defined length 203 to generate final construct 643. The final construct 643 comprises the second complementary region adjacent to a second variable region 108 followed by the second variable region 109, the second hypervariable region 110, the second region of any defined length 203, the self-cleaving peptide sequence 104, the first complementary region adjacent to a first variable region 105, the first variable region 106, the first hypervariable region 102, and the second variable constant segment 211. In some instances, a number of final constructs generated is about 10000. In some instances, a number of final constructs generated is about 1000, 2000, 5000, 8000, 10000, 15,000, 20,000, 100,000, or about 1,000,000. In some instances, a number of final constructs generated is at least 1000, 2000, 5000, 8000, 10000, 15,000, 20,000, 100,000, or at least 1,000,000.

Described herein are methods of de novo synthesis for nucleic acid sequence assembly. Such methods are in some instances used for the assembly of smaller nucleic acid fragments. In some instances, nucleic acid fragments comprise constant regions, variable regions, overlap regions, hypervariable regions, barcodes, regions encoding for peptide cleavage sites, regions encoding for genes or fragments of genes, restriction sites, or other region. In some instances, a first constant sequence, a first variable sequence, and a first sequence are synthesized and then subject to polymerase chain assembly (PCA) to generate a first plurality of gene fragments. In some instances, the first constant sequence is a leader sequence. In some instances, the second sequence is a CDR. In some instances, the first constant sequence is a leader sequence, and the second sequence is a CDR. In some instances, a second constant sequence, a second variable sequence and a second sequence are synthesized and then subject to assembly PCR or PCA to generate a second plurality of gene fragments. In some instances, the second constant sequence is a leader sequence. In some instances, the second sequence is a CDR. In some instances a third plurality of gene fragments comprising a third constant region followed by a first complementary sequence and a fourth plurality of gene fragments comprising a variable constant segment are synthesized. In some instances, the first complementary sequence comprises a sequence complementary region adjacent to one or more variable regions. In some instances, the first complementary sequence comprises a 20-60 bp, 10-20 bp, 15-45 bp, 20-60 bp, 30-40 bp, 30-60 bp, or a 40-60 bp region. In some instances, the first complementary sequence comprises about a 15, 20, 25, 30, 35, 40, 45, 50, 55, 60, or 65 bp region. In some instances, the first complementary sequence comprises about a 40 bp region. In some instances, the first complementary sequence comprises a self-cleaving peptide. In some instances a self-cleaving peptide sequence is P2A. In some instances, the third plurality of gene fragments and the fourth plurality of gene fragments are added to the first plurality of gene fragments and the second plurality of gene fragments followed by PCR. Optionally, an error correction reaction is performed. In some instances, resulting construct is pooled, cloned, and subject to next generation sequencing. In some instances, the resulting construct comprises one or more genes. In some instances, the resulting construct comprises an immunoglobulin, or fragment thereof.

Described herein are methods of de novo synthesis for nucleic acid sequence assembly. Such methods are in some instances used for the assembly of smaller nucleic acid fragments. In some instances, nucleic acid fragments comprise constant regions, variable regions, hypervariable regions, overlap regions, barcodes, regions encoding for peptide cleavage sites, regions encoding for genes or fragments of genes, restriction sites, or other region. In some instances, nucleic acid fragments comprise gene fragments. In some instances, the fragments are at least 50, 75, 100, 125, 150, 175, 200, 250, 500, 800, 1000, 2000, 5000, 8000, 10,000, or at least 20,000 bases in length. In some instances, the fragments are no more than 50, 75, 100, 125, 150, 175, 200, 250, 500, 800, 1000, 2000, 5000, 8000, 10,000, or no more than 20,000 bases in length. In some instances, the fragments are about 50, 75, 100, 125, 150, 175, 200, 250, 500, 800, 1000, 2000, 5000, 8000, 10,000, or about 20,000 bases in length. In some instances, the fragments are 50-5000, 50-1000, 50-500, 50-250, 100-500, 200-1000, 500-10,000, 500-5,000, 1000-8000, or 1500-10,000 bases in length. Nucleic acid fragments are synthesized comprising variants of a first variable region and amplified with fragments comprising a region of fixed variability. In some instances, the region of fixed variability comprises a region complementary to the first variable region and a first hypervariable region to generate a first plurality of fragments. In some instances, the first hypervariable region comprises a CDR and J segment. In some instances, the region of fixed variability comprises a 20-60 base pair (bp), 10-20 bp, 15-45 bp, 20-60 bp, 30-40 bp, 30-60 bp, or a 40-60 bp region. In some instances, the region of fixed variability comprises about a 15, 20, 25, 30, 35, 40, 45, 50, 55, 60, or 65 bp region. In some instances, the region of fixed variability comprises about a 40 bp region. Fragments can be synthesized comprising variants of a second variable region and amplified with fragments comprising a second CDR and J segment to generate a second plurality of fragments. A third plurality of fragments can be synthesized comprising a constant region, a first complementary region adjacent to the variable regions, a first leader sequence, and a second complementary region complementary to the second variable region and a second CDR and J segment. In some instances, the first complementary sequence comprises a 20-60 bp, 10-20 bp, 15-45 bp, 20-60 bp, 30-40 bp, 30-60 bp, or a 40-60 bp region. In some instances, the first complementary sequence comprises about a 15, 20, 25, 30, 35, 40, 45, 50, 55, 60, or 65 bp region. In some instances, the first complementary sequence comprises about a 40 bp region. In some instances, the second complementary sequence comprises a 20-60 bp, 10-20 bp, 15-45 bp, 20-60 bp, 30-40 bp, 30-60 bp, or a 40-60 bp region. In some instances, the second complementary sequence comprises about a 15, 20, 25, 30, 35, 40, 45, 50, 55, 60, or 65 bp region. In some instances, the second complementary sequence comprises about a 40 bp region. Constant regions may be adjusted for the construct size. In some instances, the constant region is at least 50, 75, 100, 125, 150, 175, 200, 250, 500, 800, 1000, 2000, 5000, 8000, 10,000, or at least 20,000 bases in length. In some instances, the constant region is no more than 50, 75, 100, 125, 150, 175, 200, 250, 500, 800, 1000, 2000, 5000, 8000, 10,000, or no more than 20,000 bases in length. In some instances, the constant region is about 50, 75, 100, 125, 150, 175, 200, 250, 500, 800, 1000, 2000, 5000, 8000, 10,000, or about 20,000 bases in length. In some instances, the constant region is 50-5000, 50-1000, 50-500, 50-250, 100-500, 200-1000, 500-10,000, 500-5,000, 1000-8000, or 1500-10,000 bases in length. In some instances, the first plurality of fragments, the second plurality of fragments, and the third plurality of fragments are assembled using an enzymatic based assembly method, PCR purified, and pooled. In some instances, substantially all non-assembled fragments are purified away. In some instances, at least 90%, 95%, 97%, 98%, 99%, 99.5%, 99.9%, or at least 99.99% of the non-assembled fragments are purified away. In some instances, the final construct is cloned into large nucleic acid. In some instances, the large nucleic acid is a vector.

Described herein are methods of de novo synthesis for nucleic acid sequence assembly. Such methods are in some instances used for the assembly of smaller nucleic acid fragments. In some instances, nucleic acid fragments comprise constant regions, variable regions, hypervariable regions, overlap regions, barcodes, regions encoding for peptide cleavage sites, regions encoding for genes or fragments of genes, restriction sites, or other region. In some instances, nucleic acid fragments comprise gene fragments. In some instances, the gene fragments are variant gene fragments. In some instances fragments comprising a first variable region are synthesized. In some instances, the fragments are at least 50, 75, 100, 125, 150, 175, 200, 250, 500, 800, 1000, 2000, 5000, 8000, 10,000, or at least 20,000 bases in length. In some instances, the fragments are no more than 50, 75, 100, 125, 150, 175, 200, 250, 500, 800, 1000, 2000, 5000, 8000, 10,000, or no more than 20,000 bases in length. In some instances, the fragments are about 50, 75, 100, 125, 150, 175, 200, 250, 500, 800, 1000, 2000, 5000, 8000, 10,000, or about 20,000 bases in length. In some instances, the fragments are 50-5000, 50-1000, 50-500, 50-250, 100-500, 200-1000, 500-10,000, 500-5,000, 1000-8000, or 1500-10,000 bases in length. In some instances, the fragments are amplified with a first hypervariable segment to generate a first plurality of gene fragments. In some instances, another set of fragments comprising a second variable region are synthesized. In some instances, an different set of fragments are amplified with a second hypervariable segment to generate a second plurality of gene fragments. In some instances, the hypervariable segment comprises a CDR3 and J segment. In some instances, a third plurality of gene fragments comprising a sequence homologous to the first hypervariable segment followed by a constant region, a complementary sequence, a first leader sequence, and a region complementary to the second variable region is synthesized. In some instances, the region complementary to the second variable region is 20-60 bp, 10-20 bp, 15-45 bp, 20-60 bp, 30-40 bp, 30-60 bp, or a 40-60 in length. In some instances, the region complementary to the second variable region is about a 15, 20, 25, 30, 35, 40, 45, 50, 55, 60, or 65 bp in length. In some instances, the first complementary sequence comprises a sequence complementary region adjacent to one or more variable regions. In some instances, the first complementary sequence comprises a 20-60 bp, 10-20 bp, 15-45 bp, 20-60 bp, 30-40 bp, 30-60 bp, or a 40-60 bp region. In some instances, the first complementary sequence comprises about a 15, 20, 25, 30, 35, 40, 45, 50, 55, 60, or 65 bp region. In some instances, the first complementary sequence comprises about a 40 bp region. In some instances, the first complementary sequence comprises a self-cleaving peptide. In some instances a self-cleaving peptide sequence is P2A. In some instances, the third plurality of nucleic acids comprises 10-1000, 100-500, 50-5,000, 50-10,000, 100-1000, 200-1000, 500-10,000 or 1000-10,000 variants. In some instances, the first plurality of gene fragments, the second plurality of gene fragments, and the third plurality of gene fragments are assembled. In some instances, the first plurality of gene fragments, the second plurality of gene fragments, and the third plurality of gene fragments are assembled and cloned into a destination vector. In some instances, the final construct comprises a second leader sequence followed by the second variable region, the second hypervariable segment, the second constant region, the first complementary sequence, the first leader sequence, the first variable region, the first hypervariable segment, and the variable constant region.

Provided herein are methods for nucleic acid assembly, wherein gene fragments or genes for assembly comprise a homology sequence. In some instances, the homology sequence comprises at least or about 5, 10, 20, 30, 40, 50, 60, 70, 80, 90, 100, or more than 100 base pairs. In some instance, the number of base pairs is 40 base pairs. In some instances, the number of base pairs has a range of about 5 to 100, 10 to 90, 20 to 80, 30 to 70, or 40 to 60 base pairs.

Gene fragments described herein may comprise homology sequences. In some instances, the gene fragment or genes for assembly comprise one or more homology sequences. In some instances, the one or more homology sequences is a high diversity region. In some instances, the one or more homology sequences is complementary to a variable region. In some instances, the one or more homology sequences is a hypervariable region.

Provided herein are methods for synthesizing nucleic acids, wherein gene fragments or genes for assembly comprise a barcode. In some instances, the barcode comprises at least or about 5, 10, 20, 30, 40, 50, 60, 70, 80, 90, 100, or more than 100 base pairs. In some instances, the barcode is recognized by a restriction enzyme. In some instances, the restriction enzyme recognizes asymmetric DNA sequences. In some instances, a first population of gene fragments and a second population of gene fragments are designed having complementary barcode sequences, such that subsequent to cleavage of the nucleic acids in each population, the first population and the second population are able to anneal to each other.

Various restriction enzymes and restriction sites may be used herein. In some instances, the restriction enzyme is an endonuclease. In some instances, the restriction enzyme recognizes palindromic sequences and cleaves both strands symmetrically within the recognition sequence. In some instances, the restriction enzyme recognizes asymmetric nucleic acid sequences and cleaves both nucleic acid strands outside the recognition sequence. In some instances, the endonuclease is a Type II endonuclease. Exemplary Type II endonucleases include, but are not limited to, HhaI, HindIII, NotI, BbvCI, EcoRI, and BglI. In some instances, the endonuclease is a Type IIS endonuclease. Exemplary Type IIS endonucleases include, but are not limited to, AcuI, AlwI, BaeI, BbsI, BbvI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MboII, MlyI, MmeI, Mn1I, NmeAIII, PleI, SapI, and SfaNI.

Methods as described herein, in some embodiments, comprise synthesizing nucleic acids from genes or gene fragments that encode a self-cleaving peptide. In some instances, the self-cleaving peptide is a 2A peptide. In some instances, the 2A peptide is T2A, P2A, E2A, or F2A. In some instances, the 2A peptide is P2A.

Provided herein are methods for synthesizing nucleic acids from genes or gene fragments that encode a hypervariable region. In some instances, the hypervariable region is a complementarity-determining region (CDR). In some instances, the CDR is CDR1, CDR2, or CDR3. In some instances, the CDR is a heavy domain including, but not limited to, CDR-H1, CDR-H2, and CDR-H3. In some instances, the CDR is a light domain including, but not limited to, CDR-L1, CDR-L2, and CDR-L3.

The CDR region may have varying lengths. In some instances, the CDR region comprises at least or about 5, 10, 20, 30, 40, 50, 60, 70, 80, 90, 100, 125, 150, 175, 200, 225, 250, 275, 300, 325, 350, 375, 400, or more than 400 base pairs. In some instances, the CDR region comprises about 100 base pairs.

Composition and methods described herein may comprise gene or gene fragments comprising antigen binding sequences, such as CDRs or other sequence. In some instances, the gene fragment or genes encode a CDR region and a V segment, D segment, J segment, or a combination thereof. In some instances, the gene fragment or genes comprise a CDR region and a V segment. In some instances, the gene fragment or genes comprising a CDR region and a V segment comprises at least or about 5, 10, 20, 30, 40, 50, 60, 70, 80, 90, 100, 125, 150, 175, 200, 225, 250, 275, 300, 325, 350, 375, 400, or more than 400 base pairs. In some instances, the gene fragment or genes comprise a CDR region and a D segment. In some instances, the gene fragment or genes comprising a CDR region and a D segment comprises at least or about 5, 10, 20, 30, 40, 50, 60, 70, 80, 90, 100, 125, 150, 175, 200, 225, 250, 275, 300, 325, 350, 375, 400, or more than 400 base pairs. In some instances, the gene fragment or genes comprise a CDR region and a J segment. In some instances, the gene fragment or genes comprising a CDR region and a J segment comprises at least or about 5, 10, 20, 30, 40, 50, 60, 70, 80, 90, 100, 125, 150, 175, 200, 225, 250, 275, 300, 325, 350, 375, 400, or more than 400 base pairs. In some instances, the CDR is CDR1, CDR2, or CDR3. In some instances, the CDR is CDR3.

Methods as described herein, in some embodiments, comprise synthesizing nucleic acids from genes or gene fragments that encode a variable region. In some instances, the variable region is of an immunoglobulin. In some instances, a plurality of variant variable regions are synthesized. In some instances, at least or about 10, 10¹, 10², 10³, 10⁴, 10⁵, 10⁶, or more than 10⁶ variant variable regions are synthesized. In some instances, at least or about 10, 20, 30, 40, 50, 60, 70, 80, 90, 100, 110, 120, 130, 140, 150, 160, 170, 180, 190, 200, or more than 200 variant variable regions are synthesized.

Methods as described herein, in some embodiments, comprise synthesizing nucleic acids from genes or gene fragments that encode a region of any defined length. In some instances, the region of any defined length is a constant region. In some instances, the constant region is of an immunoglobulin. In some instances, at least or about 10, 10¹, 10², 10³, 10⁴, 10⁵, 10⁶, or more than 10⁶ variant regions of any defined length are synthesized. In some instances, at least or about 10, 20, 30, 40, 50, 60, 70, 80, 90, 100, 110, 120, 130, 140, 150, 160, 170, 180, 190, 200, or more than 200 variant regions of any defined length are synthesized. In some instances, the constant region is at least 50, 75, 100, 125, 150, 175, 200, 250, 500, 800, 1000, 2000, 5000, 8000, 10,000, or at least 20,000 bases in length. In some instances, the constant region is no more than 50, 75, 100, 125, 150, 175, 200, 250, 500, 800, 1000, 2000, 5000, 8000, 10,000, or no more than 20,000 bases in length. In some instances, the constant region is about 50, 75, 100, 125, 150, 175, 200, 250, 500, 800, 1000, 2000, 5000, 8000, 10,000, or about 20,000 bases in length. In some instances, the constant region is 50-5000, 50-1000, 50-500, 50-250, 100-500, 200-1000, 500-10,000, 500-5,000, 1000-8000, or 1500-10,000 bases in length.

Provided herein are methods for nucleic acid assembly, wherein a number of gene fragments are assembled. In some instances, the gene fragments are assembled processively or sequentially. In some instances, the gene fragments are assembled into a vector. In some instances, the gene fragments are assembled for long linear gene assembly. In some instances, the number of gene fragments is at least or about 2, 3, 4, 5, 6, 7, 8, 9, 10, or more than 10 gene fragments. In some instances, the number of gene fragments is at least or about 10, 11, 12, 13, 14, 15, 16, 17, 18, 19, 20, or more than 20 gene fragments. In some instances, the number of gene fragments is in a range of about 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10. In some instances, the number of gene fragments is about 1 to about 20, about 2 to about 18, about 3 to about 17, about 4 to about 16, about 6 to about 14, or about 8 to about 12.

Provided herein are methods for nucleic acid assembly, wherein a ratio of gene fragments assembled is about 0.2:1, 0.25:1, 0.5:1, 0.75:1, 1:1, 1:1.5, 1:2, 1:3, 1:4, 1:5, or more than 1:5. For example, if two gene fragments are assembled, a ratio of the first gene fragment to the second gene fragment is 1:1. In some instances, a ratio of the first gene fragment to the second gene fragment is at least or about 1:1, 1:0.9, 1:0.85, 1:0.8, 1:0.75, 1:0.7, 1:0.65, 1:0.6, 1:0.55, 1:0.5, 1:0.45, 1:0.4, 1:0.35, 1:0.3, 1:0.25, 1:0.2, 1:0.15, 1:0.1, or less than 1:0.1.

Methods as described herein for nucleic acid assembly may comprise assembly of one or more gene fragments into a vector, wherein a ratio of the one or more gene fragments to the vector varies. In some instances, a ratio of the one or more gene fragments to the vector is at least or about 0.2:1, 0.25:1, 0.5:1, 0.75:1, 1:1, 1:1.5, 1:2, 1:3, 1:4, 1:5, or more than 1:5. In some instances, a ratio of the one or more gene fragments to the vector is at least or about 1:1, 1:0.9, 1:0.85, 1:0.8, 1:0.75, 1:0.7, 1:0.65, 1:0.6, 1:0.55, 1:0.5, 1:0.45, 1:0.4, 1:0.35, 1:0.3, 1:0.25, 1:0.2, 1:0.15, 1:0.1, or less than 1:0.1.

Methods as described herein for nucleic acid assembly may comprise assembly of polynucleotide populations for assembly into a vector. In some instances, PCR is performed for assembly of polynucleotide populations. In some instances, the polynucleotide population comprises at least or about 2, 3, 4, 5, 6, 7, 8, 9, 10, 15, 20, 25, 30, 35, 40, 45, 50, 60, 70, 80, 90, 100, 200, or more than 200 polynucleotides. In some instances, the polynucleotide population are assembled to generate a long nucleic acid comprising at least or about 50, 100, 200, 250 300, 400, 500, 600, 700, 800, 900, 1000, 1100, 1300, 1400, 1500, 1600, 1700, 1800, 2000, 2400, 2600, 2800, 3000, 3200, 3400, 3600, 3800, 4000, 4200, 4400, 4600, 4800, 5000, 6000, 7000, 8000, 9000, 10000, 20000, 30000, 40000, 50000, 60000, 70000, 80000, 90000, 100000 or more than 100000 bases.

Nucleic acid assembly, in some embodiments, result in generation of nucleic acids encoding an immunoglobulin. In some instances, the immunoglobulin is an antibody. As used herein, the term antibody will be understood to include proteins having the characteristic two-armed, Y-shape of a typical antibody molecule as well as one or more fragments of an antibody that retain the ability to specifically bind to an antigen. Exemplary antibodies include, but are not limited to, a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv) (including fragments in which the VL and VH are joined using recombinant methods by a synthetic or natural linker that enables them to be made as a single protein chain in which the VL and VH regions pair to form monovalent molecules, including single chain Fab and scFab), a single chain antibody, a Fab fragment (including monovalent fragments comprising the VL, VH, CL, and CH1 domains), a F(ab′)2 fragment (including bivalent fragments comprising two Fab fragments linked by a disulfide bridge at the hinge region), a Fd fragment (including fragments comprising the VH and CH1 fragment), a Fv fragment (including fragments comprising the VL and VH domains of a single arm of an antibody), a single-domain antibody (dAb or sdAb) (including fragments comprising a VH domain), an isolated complementarity determining region (CDR), a diabody (including fragments comprising bivalent dimers such as two VL and VH domains bound to each other and recognizing two different antigens), a fragment comprised of only a single monomeric variable domain, disulfide-linked Fvs (sdFv), an intrabody, an anti-idiotypic (anti-Id) antibody, or ab antigen-binding fragments thereof. In some instances, the libraries disclosed herein comprise nucleic acids encoding for a scaffold, wherein the scaffold is a Fv antibody, including Fv antibodies comprised of the minimum antibody fragment which contains a complete antigen-recognition and antigen-binding site. In some embodiments, the Fv antibody consists of a dimer of one heavy chain and one light chain variable domain in tight, non-covalent association, and the three hypervariable regions of each variable domain interact to define an antigen-binding site on the surface of the VH-VL dimer. In some embodiments, the six hypervariable regions confer antigen-binding specificity to the antibody. In some embodiments, a single variable domain (or half of an Fv comprising only three hypervariable regions specific for an antigen, including single domain antibodies isolated from camelid animals comprising one heavy chain variable domain such as VHH antibodies or nanobodies) has the ability to recognize and bind antigen. In some instances, the libraries disclosed herein comprise nucleic acids encoding for a scaffold, wherein the scaffold is a single-chain Fv or scFv, including antibody fragments comprising a VH, a VL, or both a VH and VL domain, wherein both domains are present in a single polypeptide chain. In some embodiments, the Fv polypeptide further comprises a polypeptide linker between the VH and VL domains allowing the scFv to form the desired structure for antigen binding. In some instances, a scFv is linked to the Fc fragment or a VHH is linked to the Fc fragment (including minibodies). In some instances, the antibody comprises immunoglobulin molecules and immunologically active fragments of immunoglobulin molecules, e.g., molecules that contain an antigen binding site. Immunoglobulin molecules are of any type (e.g., IgG, IgE, IgM, IgD, IgA and IgY), class (e.g., IgG 1, IgG 2, IgG 3, IgG 4, IgA 1 and IgA 2) or subclass.

Methods as described herein for nucleic acid assembly may comprise synthesis of gene fragments in individual reactions. In some instances, synthesis of gene fragments is followed by multiplexed gene assembly. In some instances, multiplexed gene assembly results in at least or about 2, 3, 4, 5, 6, 7, 8, 9, 10, 15, 20, 25, 30, 35, 40, 45, 50, 60, 70, 80, 90, 100, 200, 300, 400, 500, 600, 700, 800, 900, 1000, 2000, 3000, 4000, 5000, 6000, 7000, 8000, 9000, 10000, 20000, 40000, or more than 40000 sequences or gene fragments assembled. In some instances, at least or about 10, 15, 20, 25, 30, 35, 40, 45, 50, 55, 60, 65, 70, 75, 80, 85, 90, 95, 100, or more than 100 genes are assembled. In some instances, multiplexed gene assembly results in assembly of at least or about 2, 3, 4, 5, 6, 7, 8, 9, 10, 15, 20, 25, 30, 35, 40, 45, 50, 60, 70, 80, 90, 100, 200, 300, 400, 500, 600, 700, 800, or more than 800 base pairs (bp).

Nucleic acid assembly using methods as described herein may result in libraries of nucleic acids comprising low error rate, low dropout rate, low runaway, low percentage of chimeric genes, or a combination thereof. In some instances, libraries of nucleic acids assembled using methods described herein comprise base insertion, deletion, substitution, or total error rates that are under 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less, across the library, or across more than 80%, 85%, 90%, 93%, 95%, 96%, 97%, 98%, 99%, 99.5%, 99.8%, 99.9%, 99.95%, 99.98%, 99.99%, or more of the library. In some instances, libraries of nucleic acids assembled using methods described herein result in less than 1.0%, 1.5%, 2.0%, 2.5%, 3.0%, 3.5%, 4.0%, 4.5%, 5.0%, 6.0%, 6.5%, 7.0%, 7.5%, 8.0%, 8.5%, 9.0%, 9.5%, or 10% AT dropout. In some instances, libraries of nucleic acids assembled using methods described herein result in less than 1.0%, 1.5%, 2.0%, 2.5%, 3.0%, 3.5%, 4.0%, 4.5%, or 5.0% AT dropout. In some instances, libraries of nucleic acids assembled using methods described herein result in less than 1.0%, 1.5%, 2.0%, 2.5%, 3.0%, 3.5%, 4.0%, 4.5%, 5.0%, 6.0%, 6.5%, 7.0%, 7.5%, 8.0%, 8.5%, 9.0%, 9.5%, or 10% GC dropout. In some instances, libraries of nucleic acids assembled using methods described herein result in less than 1.0%, 1.5%, 2.0%, 2.5%, 3.0%, 3.5%, 4.0%, 4.5%, or 5.0% GC dropout. In some instances, libraries of nucleic acids assembled using methods described herein comprise at most 1.0%, 1.5%, 2.0%, 2.5%, 3.0%, 3.5%, 4.0%, 4.5%, 5.0%, 6.0%, 6.5%, 7.0%, 7.5%, 8.0%, 8.5%, 9.0%, 9.5%, or 10% of chimeric genes.

Methods as described herein for nucleic acid assembly may comprise enzymatic based assembly of one or more gene fragments. In some instances, the enzymatic mediated nucleic acid assembly comprises addition of homologous sequences to gene fragments. In some instances, de novo synthesized gene fragments already comprise homology sequences. In some instances, the enzymatic mediated nucleic acid assembly comprises use of an enzymatic mixture. In some instances, the enzymatic mixture comprises an endonuclease. In some instances, the enzymatic mixture optionally comprises an exonuclease, a polymerase, or a ligase. In some instances, the enzymatic mixture comprises an exonuclease, an endonuclease, a polymerase, and a ligase. In some instances, the enzymatic mixture comprises an endonuclease, a polymerase, and a ligase. In some instances, the endonuclease is a flap endonuclease. In some instances, enzymatic mediated nucleic acid assembly results in improved efficiency. In some instances, the enzymatic mixture comprises enzymes that are not restriction enzymes. In some instances, the enzymatic mixture comprises enzymes that are structure specific enzymes. In some instances, the enzymatic mixture comprises enzymes that are structure specific enzymes and not sequence specific enzymes.

Methods for enzymatic mediated nucleic acid assembly, in some embodiments, comprise contacting a nucleic acid using an enzyme comprising exonuclease activity. In some instances, the exonuclease comprises 3′ exonuclease activity. Exemplary exonucleases comprising 3′exonuclease activity include, but are not limited to, exonuclease I, exonuclease III, exonuclease V, exonuclease VII, and exonuclease T. In some instances, the exonuclease comprises 5′ exonuclease activity. Exemplary exonucleases comprising 5′ exonuclease activity include, but are not limited to, exonuclease II, exonuclease IV, exonuclease V, exonuclease VI, exonuclease VII, exonuclease VIII, T5 exonuclease, and T7 exonuclease. In some instances, the exonuclease is exonuclease III (ExoIII). Exonucleases include wild-type exonucleases and derivatives, chimeras, and/or mutants thereof. Mutant exonucleases include enzymes comprising one or more mutations, insertions, deletions or any combination thereof within the amino acid or nucleic acid sequence of an exonuclease.

In some instances, the exonuclease is used at a temperature optimal for enzymatic activity, for example, a temperature in a range of about 25-80° C., 25-70° C., 25-60° C., 25-50° C., or 25-40° C. In some instances, the temperature is about 37° C. In some instances, the temperature is about 50° C. In some instances, the temperature is about 55° C. In some instances, the temperature is about 65° C. In some instances, the temperature is at least or about 15° C., 20° C., 25° C., 30° C., 35° C., 40° C., 45° C., 50° C., 55° C., 60° C., 65° C., 70° C., 75° C., 80° C., or more than 80° C.

In some instances, methods for enzymatic mediated nucleic acid assembly do not comprise using an exonuclease. In some instances, methods for enzymatic mediated nucleic acid assembly comprise using an exonuclease. In some instances, one or more exonucleases are used. For example, at least or about 1, 2, 3, 4, 5, 6, or more than 6 exonucleases are used. In some instances, the exonuclease comprises 5′ to 3′ exonuclease activity. In some instances, the exonuclease comprises 3′ to 5′ exonuclease activity. In some instances, methods comprise contacting double stranded DNA with an endonuclease. In some instances, the endonuclease is a flap endonuclease. In some instances, methods comprise contacting double stranded DNA with a flap endonuclease, a ligase, or a polymerase. In some instances, the flap endonuclease is flap endonuclease 1.

Methods for enzymatic mediated nucleic acid assembly, in some embodiments, comprise contacting a nucleic acid using an enzyme comprising endonuclease activity. In some instances, the endonuclease comprises 5′ nuclease activity. In some instances, the endonuclease comprises 3′ nuclease activity. In some instances, the endonuclease is a flap endonuclease. In some instances, the flap endonuclease comprises 5′ nuclease activity. In some instances, the flap endonuclease is a member of a 5′-nuclease family of enzymes. Exemplary 5′-nuclease enzymes include, but are not limited to, flap endonuclease 1, exonuclease 1, xeroderma pigmentosum complementation group G (XPG), Dna2, and gap endonuclease 1 (GEN1). In some instances, the flap endonuclease is flap endonuclease 1. In some instances, the flap endonuclease comprises 3′ nuclease activity. Exemplary flap endonucleases with 3′ nuclease activity include, but are not limited to, RAG1, RAG2, and MUS81. In some instances, the flap endonuclease is an archaeal, bacteria, yeast, plant, or mammalian flap endonuclease.

In some instances, the endonuclease is used at a temperature optimal for enzymatic activity, for example, a temperature of 25-80° C., 25-70° C., 25-60° C., 25-50° C., or 25-40° C. In some instances, the temperature is about 50° C. In some instances, the temperature is about 55° C. In some instances, the temperature is about 65° C. In some instances, the temperature is at least or about 15° C., 20° C., 25° C., 30° C., 35° C., 40° C., 45° C., 50° C., 55° C., 60° C., 65° C., 70° C., 75° C., 80° C., or more than 80° C. In some instances, the endonuclease is a thermostable endonuclease. A thermostable endonuclease may include endonucleases that are functional at temperatures at least or about 60° C., 65° C., 70° C., 75° C., 80° C., or more than 80° C. In some instances, the endonuclease is a flap endonuclease. In some instances, the flap endonuclease is a thermostable flap endonuclease.

Provided herein are methods for nucleic acid assembly, wherein the ratio of the endonuclease to the exonuclease is from about 0.1:1 to about 1:5. In some instances, the endonuclease is a flap endonuclease. In some instances, the ratio of the endonuclease to the exonuclease is at least or about 0.2:1, 0.25:1, 0.5:1, 0.75:1, 1:1, 1:1.5, 1:2, 1:3, 1:4, 1:5, or more than 1:5. In some instances, the ratio of the endonuclease to the exonuclease is at least or about 1:1, 1:0.9, 1:0.85, 1:0.8, 1:0.75, 1:0.7, 1:0.65, 1:0.6, 1:0.55, 1:0.5, 1:0.45, 1:0.4, 1:0.35, 1:0.3, 1:0.25, 1:0.2, 1:0.15, 1:0.1, or less than 1:0.1.

Provided herein are methods for nucleic acid assembly comprising an exonuclease, wherein the concentration of the exonuclease is from about 0.1U to about 20U or more. For example, the concentration of the exonuclease is at least or about 0.1U, 0.25U, 0.5U, 0.75U, 1U, 1.6U, 2U, 3U, 4U, 5U, 6U, 7U, 8U, 9U, 10U, 12U, 14U, 16U, 18U, 20U, or more than 20U. In some instances, the concentration of the exonuclease is in a range of about 0.5U to about 1.0U. In some instances, the concentration of the exonuclease is from about 1.0U to about 2.0U. In some instances, the concentration of the exonuclease is about 1.6U. In some instances, the concentration of the exonuclease is about 5.0U. In some instances, the concentration of the exonuclease from about 0.1U to 20U, 0.25U to 18U, 0.5U to 16U, 0.75U to 14U, 1U to 12U, 2U to 10U, 3U to 9U, or 4U to 8U.

Methods described herein for enzymatic mediated nucleic acid assembly may comprise an endonuclease, wherein the concentration of the endonuclease is from about 0.25U to about 12U or more. In some instances, the endonuclease is a flap endonuclease. Exemplary concentrations of the endonuclease, include, but are not limited to, at least or about 0.25U, 0.5U, 0.75U, 1U, 2U, 3U, 4U, 5U, 6U, 7U, 8U, 9U, 10U, 11U, 12U, or more than 12U. In some instances, the concentration of the endonuclease is 0.32U. In some instances, the concentration of the endonuclease is 1.6U. In some instances, the concentration of the endonuclease is in a range of about 0.32U to about 4.8U. In some instances, the concentration of the endonuclease is in a range of about 0.25U to 12U, 0.5U to 11U, 0.75U to 10U, 1U to 9U, 2U to 8U, 3U to 7U, or 4U to 6U.

Provided herein are methods for enzymatic mediated nucleic acid assembly, wherein a nucleic acid is mixed with a polymerase. In some instances, the polymerase is a DNA polymerase. In some instances, the polymerase is a high fidelity polymerase. A high fidelity polymerase may include polymerases that result in accurate replication or amplification of a template nucleic acid. In some instances, the DNA polymerase is a thermostable DNA polymerase. The DNA polymerase may be from any family of DNA polymerases including, but not limited to, Family A polymerase, Family B polymerase, Family C polymerase, Family D polymerase, Family X polymerase, and Family Y polymerase. In some instances, the DNA polymerase is from a genus including, but not limited to, Thermus, Bacillus, Thermococcus, Pyrococcus, Aeropyrum, Aquifex, Sulfolobus, Pyrolobus, or Methanopyrus.

Polymerases described herein for use in an amplification reaction may comprise various enzymatic activities. Polymerases are used in the methods of the invention, for example, to extend primers to produce extension products. In some instances, the DNA polymerase comprises 5′ to 3′ polymerase activity. In some instances, the DNA polymerase comprises 3′ to 5′ exonuclease activity. In some instances, the DNA polymerase comprises proofreading activity. Exemplary polymerases include, but are not limited to, DNA polymerase (I, II, or III), T4 DNA polymerase, T7 DNA polymerase, Bst DNA polymerase, Bca polymerase, Vent DNA polymerase, Pfu DNA polymerase, and Taq DNA polymerase. Non-limiting examples of thermostable DNA polymerases include, but are not limited to, Taq, Phusion® DNA polymerase, Q5C) High Fidelity DNA Polymerase, LongAmp® DNA polymerase, Expand High Fidelity polymerase, HotTub polymerase, Pwo polymerase, Tfl polymerase, Tli polymerase, UlTma polymerase, Pfu polymerase, KOD DNA polymerase, JDF-3 DNA polymerase, PGB-D DNA polymerase, Tgo DNA polymerase, Pyrolobus furmarius DNA polymerase, Vent polymerase, and Deep Vent polymerase.

Described herein are methods comprising a DNA polymerase, wherein a concentration of the DNA polymerase is from about 0.1U to about 2U, or more than 2U. In some instances, the concentration of the DNA polymerase is about 0.1U. In some instances, the concentration of the DNA polymerase is about 0.2U. In some instances, the concentration of the DNA polymerase is about 0.01U. In some instances, the concentration of the DNA polymerase is in a range of at least or about 0.005U to 2U, 0.005U to 1U, 0.005U to 0.5U, 0.01U to 1U, 0.1U to 0.5U, 0.1U to 0.5U, 0.1U to 1U, 0.1U to 1.5U, 0.1U to 2U, 0.5U to 1.0U, 0.5U to 1.5U, 0.5U to 2U, 1U to 1.5U, 1.0U to 2.0U, or 1.5U to 2U.

The DNA polymerase for use in methods described herein are used at a temperature optimal for enzymatic activity, for example, a temperature of 25-80° C., 25-70° C., 25-60° C., 25-50° C., or 25-40° C. In some instances, the temperature is about 50° C. In some instances, the temperature is about 55° C. In some instances, the temperature is about 65° C. In some instances, the temperature is at least or about 15° C., 20° C., 25° C., 30° C., 35° C., 40° C., 45° C., 50° C., 55° C., 60° C., 65° C., 70° C., 75° C., 80° C., or more than 80° C.

Methods for enzymatic mediated nucleic acid assembly as described herein, in some embodiments, comprise treating a nucleic acid using a ligase. Ligases as described herein may function to join nucleic acid fragments. For example, the ligase functions to join adjacent 3′-hydroxylated and 5′-phosphorylated termini of DNA. Ligases include, but are not limited to, E. coli ligase, T4 ligase, mammalian ligases (e.g., DNA ligase I, DNA ligase II, DNA ligase III, DNA ligase IV), thermostable ligases, and fast ligases. In some instances, the ligase is a thermostable ligase. In some instances, the ligase is Ampligase.

The concentration of the ligase may vary. In some instances, the concentration of the ligase is in a range of about 0U to about 2U. An exemplary concentration of the ligase is about 0.5U. In some instances, the concentration of the ligase is about 1.0U. In some instances, the concentration of the ligase is about 5.0U. In some instances, the concentration of the ligase is in a range of at least or about 0U to 0.25U, 0U to 0.5U, 0U to 1U, 0U to 1.5U, 0U to 2U, 0.25U to 0.5U, 0.25U to 1.0U, 0.25U to 1.5U, 0.25U to 2.0U, 0.5U to 1.0U, 0.5U to 1.5U, 0.5U to 2.0U, 1.0U to 1.5U, 1.0U to 2.0U, or 1.5U to 2.0U, 2.0U to 4.0U, 4.0U to 6.0U, 4.0U to 8.0U, 6.0U to 10.0U.

In some instances, the ligase is used at a temperature optimal for enzymatic activity, for example, a temperature of 25-80° C., 25-70° C., 25-60° C., 25-50° C., or 25-40° C. In some instances, the temperature is about 50° C. In some instances, the temperature is about 55° C. In some instances, the temperature is about 65° C. In some instances, the temperature is at least or about 15° C., 20° C., 25° C., 30° C., 35° C., 40° C., 45° C., 50° C., 55° C., 60° C., 65° C., 70° C., 75° C., 80° C., or more than 80° C.

Methods described herein for nucleic acid assembly may comprise a ligation reaction. One example of a ligation reaction is polymerase chain assembly (PCA). In some instances, at least of a portion of the polynucleotides are designed to include an appended region that is a substrate for universal primer binding. For PCA reactions, the presynthesized polynucleotides include overlaps with each other (e.g., 4, 20, 40 or more bases with overlapping sequence). During the polymerase cycles, the polynucleotides anneal to complementary fragments and then are filled in by polymerase. Each cycle thus increases the length of various fragments randomly depending on which polynucleotides find each other. Complementarity amongst the fragments allows for forming a complete large span of double-stranded DNA. In some instances, after the PCA reaction is complete, an error correction step is conducted using mismatch repair detecting enzymes to remove mismatches in the sequence.

In some instances, methods described herein comprise an amplification reaction. In some instances, the amplification reaction is polymerase chain reaction (PCR). In some instances, the amplification reaction is dial-out PCR. In some instances, the amplification reaction comprises hybridization of a universal primer binding sequence during amplification. In some instances, the universal primer binding sequence is capable of binding the same 5′ or 3′ primer. In some instances, the universal primer binding sequence is shared among a plurality of target nucleic acids in the amplification reaction.

Provided herein are methods for nucleic acid assembly that may comprise an error correction step. Error correction may be performed on synthesized polynucleotides and/or assembled products. An example strategy for error correction involves site-directed mutagenesis by overlap extension PCR to correct errors, which is optionally coupled with two or more rounds of cloning and sequencing. In certain instances, double-stranded nucleic acids with mismatches, bulges and small loops, chemically altered bases and/or other heteroduplexes are selectively removed from populations of correctly synthesized nucleic acids. In some instances, error correction is performed using proteins/enzymes that recognize and bind to or next to mismatched or unpaired bases within double-stranded nucleic acids to create a single or double-strand break or to initiate a strand transfer transposition event. Non-limiting examples of proteins/enzymes for error correction include endonucleases (T7 Endonuclease I, E. coli Endonuclease V, T4 Endonuclease VII, mung bean nuclease, Cell, E. coli Endonuclease IV, UVDE), restriction enzymes, glycosylases, ribonucleases, mismatch repair enzymes, resolvases, helicases, ligases, antibodies specific for mismatches, and their variants. Examples of specific error correction enzymes include T4 endonuclease 7, T7 endonuclease 1, S1, mung bean endonuclease, MutY, MutS, MutH, MutL, cleavase, CELI, and HINF1. In some instances, DNA mismatch-binding protein MutS (Thermus aquaticus) is used to remove failure products from a population of synthesized products. In some instances, error correction is performed using the enzyme Correctase. In some instances, error correction is performed using SURVEYOR endonuclease (Transgenomic), a mismatch-specific DNA endonuclease that scans for known and unknown mutations and polymorphisms for heteroduplex DNA.

The resulting nucleic acids can be verified. In some cases, the nucleic acids are verified by sequencing. In some instances, the nucleic acids are verified by high-throughput sequencing such as by next generation sequencing. Sequencing of the sequencing library can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis.

Methods as described herein, in some embodiments, result in generation of libraries comprising at least or about 10¹, 10², 10³, 10⁴, 10⁵, 10⁶, 10⁷, 10⁸, 10⁹, 10¹⁰, or more than 10¹⁰ variants. In some instances, sequences for each variant of the libraries comprising at least or about 10¹, 10², 10³, 10⁴, 10⁵, 10⁶, 10⁷, 10⁸, 10⁹, or 10¹⁰ variants are known. In some instances, the libraries comprise a predicted diversity of variants. In some instances, the diversity represented in the libraries is at least or about 60%, 65%, 70%, 75%, 80%, 85%, 90%, 95%, or more than 95% of the predicted diversity. In some instances, the diversity represented in the libraries is at least or about 70% of the predicted diversity. In some instances, the diversity represented in the libraries is at least or about 80% of the predicted diversity. In some instances, the diversity represented in the libraries is at least or about 90% of the predicted diversity. In some instances, the diversity represented in the libraries is at least or about 99% of the predicted diversity. As described herein the term “predicted diversity” refers to a total theoretical diversity in a population comprising all possible variants.

Nucleic acid assembly using methods as described herein may efficiently assemble fragments despite high GC content, direct repeats, or secondary structures. In some instances, the fragments for assembly comprise GC content of at least or about 5%, 10%, 15%, 20%, 25%, 30%, 35%, 40%, 45%, 50%, 60%, 65%, 70%, 75%, 80%, 85%, 90%, 95%, or more than 95%. In some instances, the fragments for assembly comprise at least or about 10, 15, 20, 25, 30, 35, 40, 45, 50, 55, 60, 65, 70, 75, or 80 base pairs (bp) adjacent direct repeats. In some instances, the fragments for assembly comprise secondary structures such as hairpin structures with dG values of at least or about −5, −6, −7, −8, −9, −10, −11, −12, −13, −14, −15, −16, −17, −18, −19, −20, −21, −22, −23, −24, −25, or −26 dG. In some instances, the fragments for assembly comprise secondary structures such as hairpin structures with dG values in a range of about −11 to about −18 dG.

Provided herein are methods for assembly of highly uniform libraries of nucleic acids. In some cases, more than about 80% of synthesized of nucleic acids (RNA or DNA) are represented within 5× of the mean for of nucleic acid representation for a nucleic acid library. In some cases, more than about 90% of synthesized of nucleic acids (RNA or DNA) are represented within 5× of the mean for of nucleic acid representation for a nucleic acid library. In some cases, more than about 90% of nucleic acids are represented within 2× of the mean for nucleic acid representation for the library. In some cases, more than about 90% of nucleic acids are represented within 1.5× of the mean for nucleic acid representation for the library. In some cases, more than about 80% of nucleic acids are represented within 1.5× of the mean for nucleic acid representation for the library.

Nucleic acid libraries assembled by methods described herein comprise a high percentage of correct sequences compared to predetermined sequences. In some instances, nucleic acids libraries disclosed herein have greater than 70% correct sequence compared to predetermined sequences for nucleic acids. In some instances, nucleic acids libraries disclosed herein have greater than 75% correct sequence compared to predetermined sequences for the nucleic acids. In some instances, nucleic acids libraries disclosed herein have greater than 80% correct sequence compared to predetermined sequences for the nucleic acids. In some instances, nucleic acids libraries disclosed herein have greater than 85% correct sequence compared to predetermined sequences for the nucleic acids. In some instances, nucleic acids libraries disclosed herein have greater than 90% correct sequence compared to predetermined sequences for the nucleic acids. In some instances, nucleic acids libraries disclosed herein have greater than 95% correct sequence compared to predetermined sequences for the nucleic acids. In some instances, nucleic acids libraries disclosed herein have greater than 100% correct sequence compared to predetermined sequences for the nucleic acids.

In some instances, nucleic acids libraries disclosed herein have greater than 70% correct sequence compared to predetermined sequences for the nucleic acids following an amplification reaction. In some instances, nucleic acids libraries disclosed herein have greater than 75% correct sequence compared to predetermined sequences for the nucleic acids following an amplification reaction. In some instances, nucleic acids libraries disclosed herein have greater than 80% correct sequence compared to predetermined sequences for the nucleic acids following an amplification reaction. In some instances, nucleic acids libraries disclosed herein have greater than 85% correct sequence compared to predetermined sequences for the nucleic acids following an amplification reaction. In some instances, nucleic acids libraries disclosed herein have greater than 90% correct sequence compared to predetermined sequences for the nucleic acids following an amplification reaction. In some instances, nucleic acids libraries disclosed herein have greater than 95% correct sequence compared to predetermined sequences for the nucleic acids following an amplification reaction. In some instances, nucleic acids libraries disclosed herein have 100% correct sequence compared to predetermined sequences for the nucleic acids following an amplification reaction.

Provided herein are nucleic acid libraries having high uniformity following amplification. In some instances, more than 80% of nucleic acids are represented within at least about 1.5× the mean representation for the entire library following amplification. In some instances, more than 90% of nucleic acids described herein are represented within at least about 1.5× the mean representation for the entire library following amplification. In some instances, more than 80% of nucleic acids are represented within at least about 2× the mean representation for the entire library following amplification. In some instances, more than 80% of nucleic acids are represented within at least about 2× the mean representation for the entire library following amplification.

Systems for Nucleic Acid Sequence Assembly

Polynucleotide Synthesis

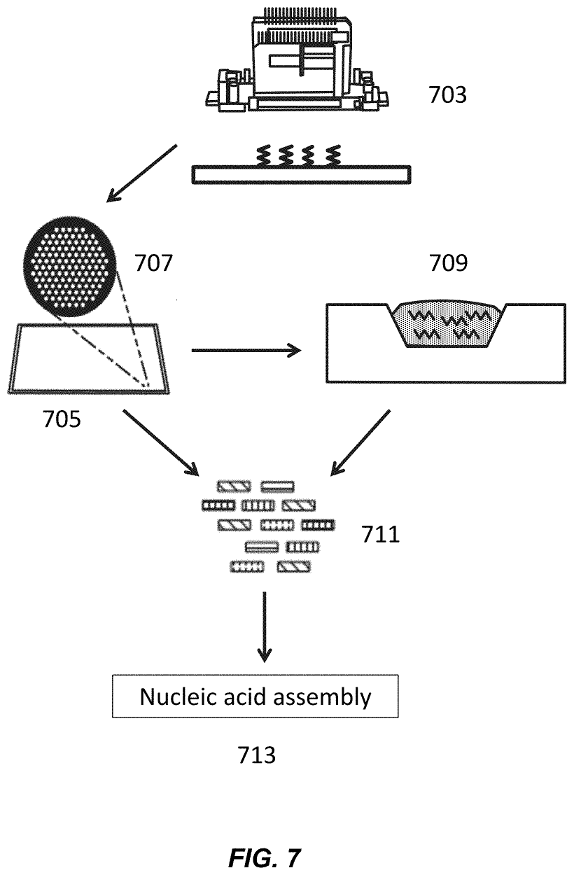

Provided herein are methods for barcode nucleic acid sequence assembly of nucleic acids following generation of polynucleotides by de novo synthesis by methods described herein. An exemplary workflow is seen in FIG. 7. A computer readable input file comprising a nucleic acid sequence is received. A computer processes the nucleic acid sequence to generate instructions for synthesis of the polynucleotide sequence or a plurality of polynucleotide sequences collectively encoding the nucleic acid sequence. Instructions are transmitted to a material deposition device 703 for synthesis of the plurality of polynucleotides based on the plurality of nucleic acid sequences. The material deposition device 703, such as a polynucleotide acid synthesizer, is designed to release reagents in a step wise fashion such that multiple polynucleotides extend, in parallel, one residue at a time to generate oligomers with a predetermined nucleic acid sequence. The material deposition device 703 generates oligomers on an array 705 that includes multiple clusters 707 of loci for polynucleotide acid synthesis and extension. However, the array need not have loci organized in clusters. For example, the loci can be uniformly spread across the array. De novo polynucleotides are synthesized and removed from the plate and an assembly reaction commenced in a collection chamber 709 followed by formation population of longer polynucleotides 711. The collection chamber may comprise a sandwich of multiple surfaces (e.g., a top and bottom surface) or well or channel in containing transferred material from the synthesis surface. De novo polynucleotides can also be synthesized and removed from the plate to form a population of longer polynucleotides 711. The population of longer polynucleotides 711 can then be partitioned into droplets or subject to PCR. The population of longer polynucleotides 711 is then subject to nucleic acid assembly 713. In some instances, nucleic acid assembly comprises variant homology sequences. In some instances, nucleic acid assembly comprises paired variant assembly using paired homology sequences. In some instances, the paired variant assembly comprises a barcode. In some instances, the barcode is exposed by a restriction endonuclease such as a Type IIS restriction endonuclease.

Provided herein are systems for sequence assembly of nucleic acids following generation of polynucleotides by de novo synthesis by methods described herein. In some instances, the system comprises a computer, a material deposition device, a surface, and a nucleic acid assembly surface. In some instances, the computer comprises a readable input file with a nucleic acid sequence. In some instances, the computer processes the nucleic acid sequence to generate instructions for synthesis of the polynucleotide sequence or a plurality of polynucleotide sequences collectively encoding for the nucleic acid sequence. In some instances, the computer provides instructions to the material deposition device for the synthesis of the plurality of polynucleotide acid sequences. In some instances, the material deposition device deposits nucleosides on the surface for an extension reaction. In some instances, the surface comprises a locus for the extension reaction. In some instances, the locus is a spot, well, microwell, channel, or post. In some instances, the plurality of polynucleotide acid sequences is synthesized following the extension reaction. In some instances, the plurality of polynucleotide acid sequences is removed from the surface and prepared for nucleic acid assembly. In some instances, the nucleic acid assembly comprises barcode immunoglobulin sequence assembly.

Provided herein are methods for polynucleotide synthesis involving phosphoramidite chemistry. In some instances, polynucleotide synthesis comprises coupling a base with phosphoramidite. In some instances, polynucleotide synthesis comprises coupling a base by deposition of phosphoramidite under coupling conditions, wherein the same base is optionally deposited with phosphoramidite more than once, i.e., double coupling. In some instances, polynucleotide synthesis comprises capping of unreacted sites. In some cases, capping is optional. In some instances, polynucleotide synthesis comprises oxidation. In some instances, polynucleotide synthesis comprises deblocking or detritylation. In some instances, polynucleotide synthesis comprises sulfurization. In some cases, polynucleotide synthesis comprises either oxidation or sulfurization. In some instances, between one or each step during a polynucleotide synthesis reaction, the substrate is washed, for example, using tetrazole or acetonitrile. Time frames for any one step in a phosphoramidite synthesis method include less than about 2 min, 1 min, 50 sec, 40 sec, 30 sec, 20 sec or 10 sec.

Polynucleotide synthesis using a phosphoramidite method comprises the subsequent addition of a phosphoramidite building block (e.g., nucleoside phosphoramidite) to a growing polynucleotide chain for the formation of a phosphite triester linkage. Phosphoramidite polynucleotide synthesis proceeds in the 3′ to 5′ direction. Phosphoramidite polynucleotide synthesis allows for the controlled addition of one nucleotide to a growing nucleic acid chain per synthesis cycle. In some instances, each synthesis cycle comprises a coupling step. Phosphoramidite coupling involves the formation of a phosphite triester linkage between an activated nucleoside phosphoramidite and a nucleoside bound to the substrate, for example, via a linker. In some instances, the nucleoside phosphoramidite is provided to the substrate activated. In some instances, the nucleoside phosphoramidite is provided to the substrate with an activator. In some instances, nucleoside phosphoramidites are provided to the substrate in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition of a nucleoside phosphoramidite, the substrate is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the substrate is de-protected by removal of a protecting group, where the protecting group functions to prevent polymerization. A common protecting group is 4,4′-dimethoxytrityl (DMT).

Following coupling, phosphoramidite polynucleotide synthesis methods optionally comprise a capping step. In a capping step, the growing polynucleotide is treated with a capping agent. A capping step is useful to block unreacted substrate-bound 5′—OH groups after coupling from further chain elongation, preventing the formation of polynucleotides with internal base deletions. Further, phosphoramidites activated with 1H-tetrazole may react, to a small extent, with the O6 position of guanosine. Without being bound by theory, upon oxidation with I₂/water, this side product, possibly via O6-N7 migration, may undergo depurination. The apurinic sites may end up being cleaved in the course of the final deprotection of the polynucleotide thus reducing the yield of the full-length product. The O6 modifications may be removed by treatment with the capping reagent prior to oxidation with I₂/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the substrate is optionally washed.

In some instances, following addition of a nucleoside phosphoramidite, and optionally after capping and one or more wash steps, the substrate bound growing nucleic acid is oxidized. The oxidation step comprises oxidation of the phosphite triester into a tetracoordinated phosphate triester, a protected precursor of the naturally occurring phosphate diester internucleoside linkage. In some cases, oxidation of the growing polynucleotide is achieved by treatment with iodine and water, optionally in the presence of a weak base (e.g., pyridine, lutidine, collidine). Oxidation may be carried out under anhydrous conditions using, e.g. tert-Butyl hydroperoxide or (1S)-(+)-(10-camphorsulfonyl)-oxaziridine (CSO). In some methods, a capping step is performed following oxidation. A second capping step allows for substrate drying, as residual water from oxidation that may persist can inhibit subsequent coupling. Following oxidation, the substrate and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including but not limited to 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

In order for a subsequent cycle of nucleoside incorporation to occur through coupling, the protected 5′ end of the substrate bound growing polynucleotide is removed so that the primary hydroxyl group is reactive with a next nucleoside phosphoramidite. In some instances, the protecting group is DMT and deblocking occurs with trichloroacetic acid in dichloromethane. Conducting detritylation for an extended time or with stronger than recommended solutions of acids may lead to increased depurination of solid support-bound polynucleotide and thus reduces the yield of the desired full-length product. Methods and compositions of the invention described herein provide for controlled deblocking conditions limiting undesired depurination reactions. In some cases, the substrate bound polynucleotide is washed after deblocking. In some cases, efficient washing after deblocking contributes to synthesized polynucleotides having a low error rate.

Methods for the synthesis of polynucleotides typically involve an iterating sequence of the following steps: application of a protected monomer to an actively functionalized surface (e.g., locus) to link with either the activated surface, a linker or with a previously deprotected monomer; deprotection of the applied monomer so that it is reactive with a subsequently applied protected monomer; and application of another protected monomer for linking. One or more intermediate steps include oxidation or sulfurization. In some cases, one or more wash steps precede or follow one or all of the steps.

Methods for phosphoramidite based polynucleotide synthesis comprise a series of chemical steps. In some instances, one or more steps of a synthesis method involve reagent cycling, where one or more steps of the method comprise application to the substrate of a reagent useful for the step. For example, reagents are cycled by a series of liquid deposition and vacuum drying steps. For substrates comprising three-dimensional features such as wells, microwells, channels and the like, reagents are optionally passed through one or more regions of the substrate via the wells and/or channels.

Polynucleotides synthesized using the methods and/or substrates described herein comprise at least about 20, 30, 40, 50, 60, 70, 75, 80, 90, 100, 120, 150, 200, 500 or more bases in length. In some instances, at least about 1 pmol, 10 pmol, 20 pmol, 30 pmol, 40 pmol, 50 pmol, 60 pmol, 70 pmol, 80 pmol, 90 pmol, 100 pmol, 150 pmol, 200 pmol, 300 pmol, 400 pmol, 500 pmol, 600 pmol, 700 pmol, 800 pmol, 900 pmol, 1 nmol, 5 nmol, 10 nmol, 100 nmol or more of an polynucleotide is synthesized within a locus. Methods for polynucleotide synthesis on a surface provided herein allow for synthesis at a fast rate. As an example, at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100, 125, 150, 175, 200 nucleotides per hour, or more are synthesized. Nucleotides include adenine, guanine, thymine, cytosine, uridine building blocks, or analogs/modified versions thereof. In some instances, libraries of polynucleotides are synthesized in parallel on a substrate. For example, a substrate comprising about or at least about 100; 1,000; 10,000; 100,000; 1,000,000; 2,000,000; 3,000,000; 4,000,000; or 5,000,000 resolved loci is able to support the synthesis of at least the same number of distinct polynucleotides, wherein a polynucleotide encoding a distinct sequence is synthesized on a resolved locus.

Various suitable methods are known for generating high density polynucleotide arrays. In an exemplary workflow, a substrate surface layer is provided. In the example, chemistry of the surface is altered in order to improve the polynucleotide synthesis process. Areas of low surface energy are generated to repel liquid while areas of high surface energy are generated to attract liquids. The surface itself may be in the form of a planar surface or contain variations in shape, such as protrusions or microwells which increase surface area. In the workflow example, high surface energy molecules selected serve a dual function of supporting DNA chemistry, as disclosed in International Patent Application Publication WO/2015/021080, which is herein incorporated by reference in its entirety.

In situ preparation of polynucleotide arrays is generated on a solid support and utilizes a single nucleotide extension process to extend multiple oligomers in parallel. A deposition device, such as a polynucleotide synthesizer, is designed to release reagents in a step wise fashion such that multiple polynucleotides extend, in parallel, one residue at a time to generate oligomers with a predetermined nucleic acid sequence. In some cases, polynucleotides are cleaved from the surface at this stage. Cleavage includes gas cleavage, e.g., with ammonia or methylamine.

Substrates

Devices used as a surface for polynucleotide synthesis may be in the form of substrates which include, without limitation, homogenous array surfaces, patterned array surfaces, channels, beads, gels, and the like. Provided herein are substrates comprising a plurality of clusters, wherein each cluster comprises a plurality of loci that support the attachment and synthesis of polynucleotides. The term “locus” as used herein refers to a discrete region on a structure which provides support for polynucleotides encoding for a single predetermined sequence to extend from the surface. In some instances, a locus is on a two dimensional surface, e.g., a substantially planar surface. In some instances, a locus is on a three-dimensional surface, e.g., a well, microwell, channel, or post. In some instances, a surface of a locus comprises a material that is actively functionalized to attach to at least one nucleotide for polynucleotide synthesis, or preferably, a population of identical nucleotides for synthesis of a population of polynucleotides. In some instances, polynucleotide refers to a population of polynucleotides encoding for the same nucleic acid sequence. In some cases, a surface of a substrate is inclusive of one or a plurality of surfaces of a substrate. The average error rates for polynucleotides synthesized within a library described here using the systems and methods provided are often less than 1 in 1000, less than about 1 in 2000, less than about 1 in 3000 or less often without error correction.

Provided herein are surfaces that support the parallel synthesis of a plurality of polynucleotides having different predetermined sequences at addressable locations on a common support. In some instances, a substrate provides support for the synthesis of more than 50, 100, 200, 400, 600, 800, 1000, 1200, 1400, 1600, 1800, 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some cases, the surfaces provide support for the synthesis of more than 50, 100, 200, 400, 600, 800, 1000, 1200, 1400, 1600, 1800, 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence. In some instances, the substrate provides a surface environment for the growth of polynucleotides having at least 80, 90, 100, 120, 150, 175, 200, 225, 250, 275, 300, 325, 350, 375, 400, 425, 450, 475, 500 bases or more.

Provided herein are methods for polynucleotide synthesis on distinct loci of a substrate, wherein each locus supports the synthesis of a population of polynucleotides. In some cases, each locus supports the synthesis of a population of polynucleotides having a different sequence than a population of polynucleotides grown on another locus. In some instances, each polynucleotide sequence is synthesized with 1, 2, 3, 4, 5, 6, 7, 8, 9 or more redundancy across different loci within the same cluster of loci on a surface for polynucleotide synthesis. In some instances, the loci of a substrate are located within a plurality of clusters. In some instances, a substrate comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a substrate comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some instances, a substrate comprises about 10,000 distinct loci. The amount of loci within a single cluster is varied in different instances. In some cases, each cluster includes 1, 2, 3, 4, 5, 6, 7, 8, 9, 10, 20, 30, 40, 50, 60, 70, 80, 90, 100, 120, 130, 150, 200, 300, 400, 500 or more loci. In some instances, each cluster includes about 50-500 loci. In some instances, each cluster includes about 100-200 loci. In some instances, each cluster includes about 100-150 loci. In some instances, each cluster includes about 109, 121, 130 or 137 loci. In some instances, each cluster includes about 19, 20, 61, 64 or more loci.

In some instances, the number of distinct polynucleotides synthesized on a substrate is dependent on the number of distinct loci available on the substrate. In some instances, the density of loci within a cluster of a substrate is at least or about 1, 10, 25, 50, 65, 75, 100, 130, 150, 175, 200, 300, 400, 500, 1,000 or more loci per mm². In some cases, a substrate comprises 10-500, 25-400, 50-500, 100-500, 150-500, 10-250, 50-250, 10-200, or 50-200 mm². In some instances, the distance between the centers of two adjacent loci within a cluster is from about 10-500, from about 10-200, or from about 10-100 um. In some instances, the distance between two centers of adjacent loci is greater than about 10, 20, 30, 40, 50, 60, 70, 80, 90 or 100 um. In some instances, the distance between the centers of two adjacent loci is less than about 200, 150, 100, 80, 70, 60, 50, 40, 30, 20 or 10 um. In some instances, each locus independently has a width of about 0.5, 1, 2, 3, 4, 5, 6, 7, 8, 9, 10, 20, 30, 40, 50, 60, 70, 80, 90 or 100 um. In some cases, each locus independently has a width of about 0.5-100, 0.5-50, 10-75, or 0.5-50 um.

In some instances, the density of clusters within a substrate is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm², 3 clusters per 1 mm², 4 clusters per 1 mm², 5 clusters per 1 mm², 10 clusters per 1 mm², 50 clusters per 1 mm² or more. In some instances, a substrate comprises from about 1 cluster per 10 mm² to about 10 clusters per 1 mm². In some instances, the distance between the centers of two adjacent clusters is at least or about 50, 100, 200, 500, 1000, 2000, or 5000 um. In some cases, the distance between the centers of two adjacent clusters is between about 50-100, 50-200, 50-300, 50-500, or 100-2000 um. In some cases, the distance between the centers of two adjacent clusters is between about 0.05-50, 0.05-10, 0.05-5, 0.05-4, 0.05-3, 0.05-2, 0.1-10, 0.2-10, 0.3-10, 0.4-10, 0.5-10, 0.5-5, or 0.5-2 mm. In some cases, each cluster independently has a cross section of about 0.5 to 2, about 0.5 to 1, or about 1 to 2 mm. In some cases, each cluster independently has a cross section of about 0.5, 0.6, 0.7, 0.8, 0.9, 1, 1.1, 1.2, 1.3, 1.4, 1.5, 1.6, 1.7, 1.8, 1.9 or 2 mm. In some cases, each cluster independently has an interior cross section of about 0.5, 0.6, 0.7, 0.8, 0.9, 1, 1.1, 1.15, 1.2, 1.3, 1.4, 1.5, 1.6, 1.7, 1.8, 1.9 or 2 mm.

In some instances, a substrate is about the size of a standard 96 well plate, for example between about 100 to about 200 mm by between about 50 to about 150 mm. In some instances, a substrate has a diameter less than or equal to about 1000, 500, 450, 400, 300, 250, 200, 150, 100 or 50 mm. In some instances, the diameter of a substrate is between about 25-1000, 25-800, 25-600, 25-500, 25-400, 25-300, or 25-200 mm. In some instances, a substrate has a planar surface area of at least about 100; 200; 500; 1,000; 2,000; 5,000; 10,000; 12,000; 15,000; 20,000; 30,000; 40,000; 50,000 mm² or more. In some instances, the thickness of a substrate is between about 50-2000, 50-1000, 100-1000, 200-1000, or 250-1000 mm.

Surface Materials

Substrates, devices, and reactors provided herein are fabricated from any variety of materials suitable for the methods, compositions, and systems described herein. In certain instances, substrate materials are fabricated to exhibit a low level of nucleotide binding. In some instances, substrate materials are modified to generate distinct surfaces that exhibit a high level of nucleotide binding. In some instances, substrate materials are transparent to visible and/or UV light. In some instances, substrate materials are sufficiently conductive, e.g., are able to form uniform electric fields across all or a portion of a substrate. In some instances, conductive materials are connected to an electric ground. In some instances, the substrate is heat conductive or insulated. In some instances, the materials are chemical resistant and heat resistant to support chemical or biochemical reactions, for example polynucleotide synthesis reaction processes. In some instances, a substrate comprises flexible materials. For flexible materials, materials can include, without limitation: nylon, both modified and unmodified, nitrocellulose, polypropylene, and the like. In some instances, a substrate comprises rigid materials. For rigid materials, materials can include, without limitation: glass; fuse silica; silicon, plastics (for example polytetrafluoroethylene, polypropylene, polystyrene, polycarbonate, and blends thereof, and the like); metals (for example, gold, platinum, and the like). The substrate, solid support or reactors can be fabricated from a material selected from the group consisting of silicon, polystyrene, agarose, dextran, cellulosic polymers, polyacrylamides, polydimethylsiloxane (PDMS), and glass. The substrates/solid supports or the microstructures, reactors therein may be manufactured with a combination of materials listed herein or any other suitable material known in the art.

Surface Architecture

Provided herein are substrates for the methods, compositions, and systems described herein, wherein the substrates have a surface architecture suitable for the methods, compositions, and systems described herein. In some instances, a substrate comprises raised and/or lowered features. One benefit of having such features is an increase in surface area to support polynucleotide synthesis. In some instances, a substrate having raised and/or lowered features is referred to as a three-dimensional substrate. In some cases, a three-dimensional substrate comprises one or more channels. In some cases, one or more loci comprise a channel. In some cases, the channels are accessible to reagent deposition via a deposition device such as a polynucleotide synthesizer. In some cases, reagents and/or fluids collect in a larger well in fluid communication with one or more channels. For example, a substrate comprises a plurality of channels corresponding to a plurality of loci within a cluster, and the plurality of channels are in fluid communication with one well of the cluster. In some methods, a library of polynucleotides is synthesized in a plurality of loci of a cluster.

Provided herein are substrates for the methods, compositions, and systems described herein, wherein the substrates are configured for polynucleotide synthesis. In some instances, the structure is configured to allow for controlled flow and mass transfer paths for polynucleotide synthesis on a surface. In some instances, the configuration of a substrate allows for the controlled and even distribution of mass transfer paths, chemical exposure times, and/or wash efficacy during polynucleotide synthesis. In some instances, the configuration of a substrate allows for increased sweep efficiency, for example by providing sufficient volume for growing a polynucleotide such that the excluded volume by the growing polynucleotide does not take up more than 50, 45, 40, 35, 30, 25, 20, 15, 14, 13, 12, 11, 10, 9, 8, 7, 6, 5, 4, 3, 2, 1%, or less of the initially available volume that is available or suitable for growing the polynucleotide. In some instances, a three-dimensional structure allows for managed flow of fluid to allow for the rapid exchange of chemical exposure.

Provided herein are substrates for the methods, compositions, and systems relating to enzymatic mediated nucleic acid assembly and polynucleotide synthesis described herein, wherein the substrates comprise structures configured for housing enzymatic reactions described herein. In some instances, segregation is achieved by physical structure. In some instances, segregation is achieved by differential functionalization of the surface generating active and passive regions for polynucleotide synthesis. In some instances, differential functionalization is achieved by alternating the hydrophobicity across the substrate surface, thereby creating water contact angle effects that cause beading or wetting of the deposited reagents. Employing larger structures can decrease splashing and cross-contamination of distinct polynucleotide synthesis locations with reagents of the neighboring spots. In some cases, a device, such as a polynucleotide synthesizer, is used to deposit reagents to distinct polynucleotide synthesis locations. Substrates having three-dimensional features are configured in a manner that allows for the synthesis of a large number of polynucleotides (e.g., more than about 10,000) with a low error rate (e.g., less than about 1:500, 1:1000, 1:1500, 1:2,000; 1:3,000; 1:5,000; or 1:10,000). In some cases, a substrate comprises features with a density of about or greater than about 1, 5, 10, 20, 30, 40, 50, 60, 70, 80, 100, 110, 120, 130, 140, 150, 160, 170, 180, 190, 200, 300, 400 or 500 features per mm².

A well of a substrate may have the same or different width, height, and/or volume as another well of the substrate. A channel of a substrate may have the same or different width, height, and/or volume as another channel of the substrate. In some instances, the diameter of a cluster or the diameter of a well comprising a cluster, or both, is between about 0.05-50, 0.05-10, 0.05-5, 0.05-4, 0.05-3, 0.05-2, 0.05-1, 0.05-0.5, 0.05-0.1, 0.1-10, 0.2-10, 0.3-10, 0.4-10, 0.5-10, 0.5-5, or 0.5-2 mm. In some instances, the diameter of a cluster or well or both is less than or about 5, 4, 3, 2, 1, 0.5, 0.1, 0.09, 0.08, 0.07, 0.06, or 0.05 mm. In some instances, the diameter of a cluster or well or both is between about 1.0 and about 1.3 mm. In some instances, the diameter of a cluster or well, or both is about 1.150 mm. In some instances, the diameter of a cluster or well, or both is about 0.08 mm. The diameter of a cluster refers to clusters within a two-dimensional or three-dimensional substrate.

In some instances, the height of a well is from about 20-1000, 50-1000, 100-1000, 200-1000, 300-1000, 400-1000, or 500-1000 um. In some cases, the height of a well is less than about 1000, 900, 800, 700, or 600 um.

In some instances, a substrate comprises a plurality of channels corresponding to a plurality of loci within a cluster, wherein the height or depth of a channel is 5-500, 5-400, 5-300, 5-200, 5-100, 5-50, or 10-50 um. In some cases, the height of a channel is less than 100, 80, 60, 40, or 20 um.

In some instances, the diameter of a channel, locus (e.g., in a substantially planar substrate) or both channel and locus (e.g., in a three-dimensional substrate wherein a locus corresponds to a channel) is from about 1-1000, 1-500, 1-200, 1-100, 5-100, or 10-100 um, for example, about 90, 80, 70, 60, 50, 40, 30, 20 or 10 um. In some instances, the diameter of a channel, locus, or both channel and locus is less than about 100, 90, 80, 70, 60, 50, 40, 30, 20 or 10 um. In some instances, the distance between the center of two adjacent channels, loci, or channels and loci is from about 1-500, 1-200, 1-100, 5-200, 5-100, 5-50, or 5-30, for example, about 20 um.

Surface Modifications

Provided herein are methods for polynucleotide synthesis on a surface, wherein the surface comprises various surface modifications. In some instances, the surface modifications are employed for the chemical and/or physical alteration of a surface by an additive or subtractive process to change one or more chemical and/or physical properties of a substrate surface or a selected site or region of a substrate surface. For example, surface modifications include, without limitation, (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

In some cases, the addition of a chemical layer on top of a surface (referred to as adhesion promoter) facilitates structured patterning of loci on a surface of a substrate. Exemplary surfaces for application of adhesion promotion include, without limitation, glass, silicon, silicon dioxide and silicon nitride. In some cases, the adhesion promoter is a chemical with a high surface energy. In some instances, a second chemical layer is deposited on a surface of a substrate. In some cases, the second chemical layer has a low surface energy. In some cases, surface energy of a chemical layer coated on a surface supports localization of droplets on the surface. Depending on the patterning arrangement selected, the proximity of loci and/or area of fluid contact at the loci are alterable.

In some instances, a substrate surface, or resolved loci, onto which nucleic acids or other moieties are deposited, e.g., for polynucleotide synthesis, are smooth or substantially planar (e.g., two-dimensional) or have irregularities, such as raised or lowered features (e.g., three-dimensional features). In some instances, a substrate surface is modified with one or more different layers of compounds. Such modification layers of interest include, without limitation, inorganic and organic layers such as metals, metal oxides, polymers, small organic molecules and the like.

In some instances, resolved loci of a substrate are functionalized with one or more moieties that increase and/or decrease surface energy. In some cases, a moiety is chemically inert. In some cases, a moiety is configured to support a desired chemical reaction, for example, one or more processes in a polynucleotide acid synthesis reaction. The surface energy, or hydrophobicity, of a surface is a factor for determining the affinity of a nucleotide to attach onto the surface. In some instances, a method for substrate functionalization comprises: (a) providing a substrate having a surface that comprises silicon dioxide; and (b) silanizing the surface using, a suitable silanizing agent described herein or otherwise known in the art, for example, an organofunctional alkoxysilane molecule. Methods and functionalizing agents are described in U.S. Pat. No. 5,474,796, which is herein incorporated by reference in its entirety.

In some instances, a substrate surface is functionalized by contact with a derivatizing composition that contains a mixture of silanes, under reaction conditions effective to couple the silanes to the substrate surface, typically via reactive hydrophilic moieties present on the substrate surface. Silanization generally covers a surface through self-assembly with organofunctional alkoxysilane molecules. A variety of siloxane functionalizing reagents can further be used as currently known in the art, e.g., for lowering or increasing surface energy. The organofunctional alkoxysilanes are classified according to their organic functions.

Computer Systems

Any of the systems described herein, may be operably linked to a computer and may be automated through a computer either locally or remotely. In some instances, the methods and systems of the invention further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the invention. The computer systems may be programmed to interface between the user specified base sequence and the position of a material deposition device to deliver the correct reagents to specified regions of the substrate.

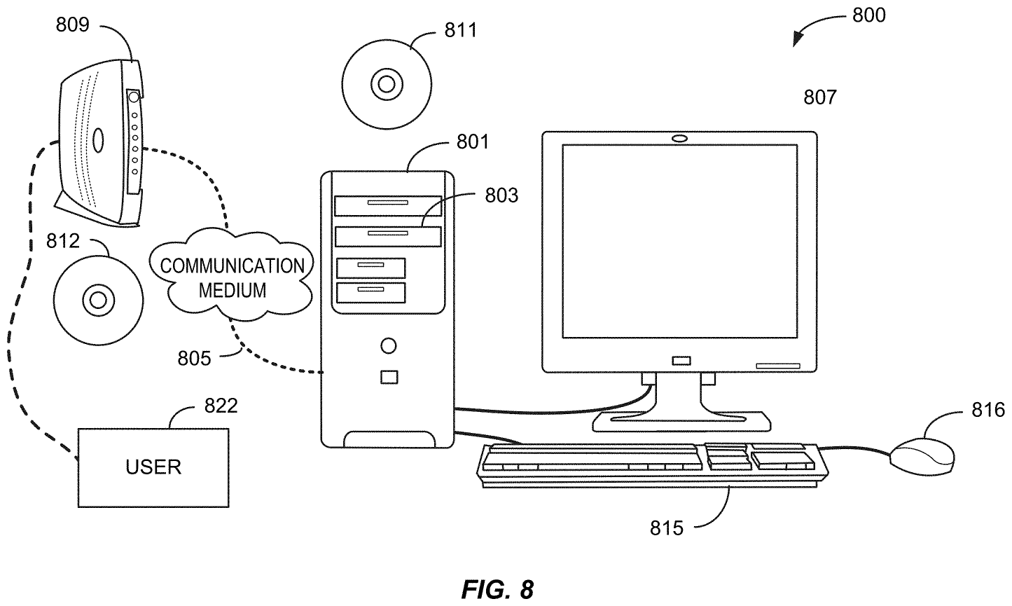

The computer system 800 illustrated in FIG. 8 may be understood as a logical apparatus that can read instructions from media 811 and/or a network port 805, which can optionally be connected to server 809 having fixed media 812. The system, such as shown in FIG. 8, can include a CPU 801, disk drives 803, optional input devices such as a keyboard 815 and/or mouse 816 and optional monitor 807. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 822 as illustrated in FIG. 8.

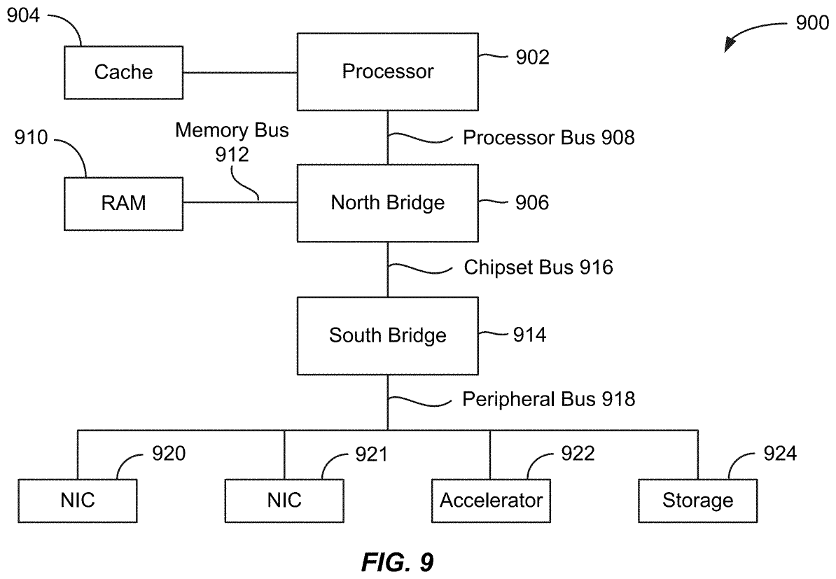

FIG. 9 is a block diagram illustrating architecture of a computer system 900 that can be used in connection with example embodiments of the present invention. As depicted in FIG. 9, the example computer system can include a processor 902 for processing instructions. Non-limiting examples of processors include: Intel Xeon™ processor, AMD Opteron processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 9, a high speed cache 904 can be connected to, or incorporated in, the processor 902 to provide a high speed memory for instructions or data that have been recently, or are frequently, used by processor 902. The processor 902 is connected to a north bridge 906 by a processor bus 908. The north bridge 906 is connected to random access memory (RAM) 910 by a memory bus 912 and manages access to the RAM 910 by the processor 902. The north bridge 906 is also connected to a south bridge 914 by a chipset bus 916. The south bridge 914 is, in turn, connected to a peripheral bus 918. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 918. In some alternative architectures, the functionality of the north bridge can be incorporated into the processor instead of using a separate north bridge chip. In some instances, system 900 can include an accelerator card 922 attached to the peripheral bus 918. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 924 and can be loaded into RAM 910 and/or cache 904 for use by the processor. The system 900 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example embodiments of the present invention. In this example, system 900 also includes network interface cards (NICs) 920 and 921 connected to the peripheral bus for providing network interfaces to external storage, such as Network Attached Storage (NAS) and other computer systems that can be used for distributed parallel processing.

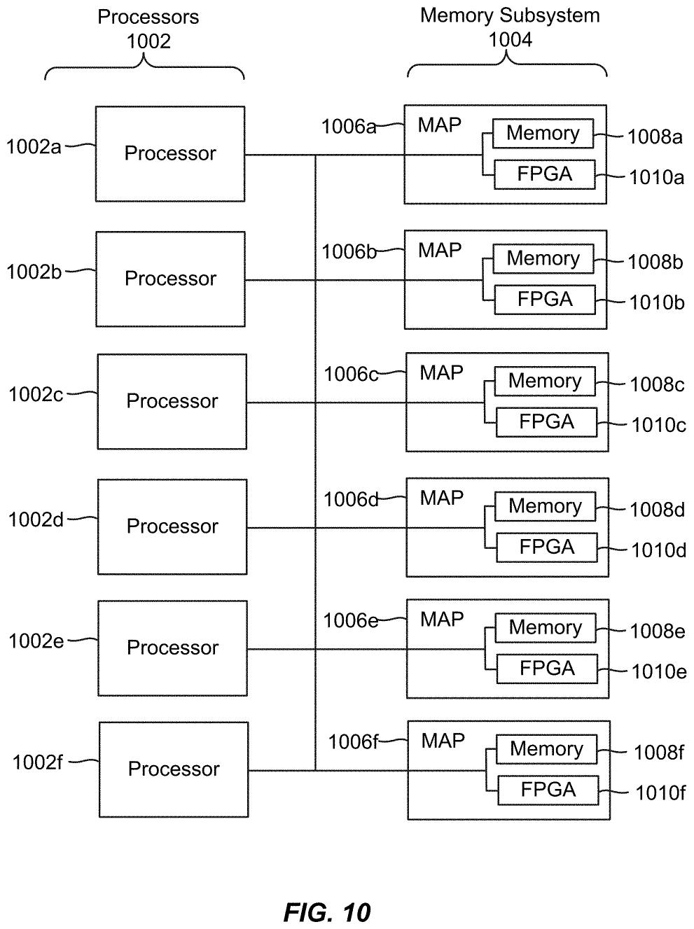

FIG. 10 is a block diagram of a multiprocessor computer system using a shared virtual address memory space in accordance with an example embodiment. The system includes a plurality of processors 1002a-f that can access a shared memory subsystem 1004. The system incorporates a plurality of programmable hardware memory algorithm processors (MAPs) 1006a-f in the memory subsystem 1004. Each MAP 1006a-f can comprise a memory 1008a-f and one or more field programmable gate arrays (FPGAs) 1010a-f. The MAP provides a configurable functional unit and particular algorithms or portions of algorithms can be provided to the FPGAs 1010a-f for processing in close coordination with a respective processor. For example, the MAPs can be used to evaluate algebraic expressions regarding the data model and to perform adaptive data restructuring in example embodiments. In this example, each MAP is globally accessible by all of the processors for these purposes. In one configuration, each MAP can use Direct Memory Access (DMA) to access an associated memory 1008a-f, allowing it to execute tasks independently of, and asynchronously from, the respective microprocessor 1002a-f. In this configuration, a MAP can feed results directly to another MAP for pipelining and parallel execution of algorithms.

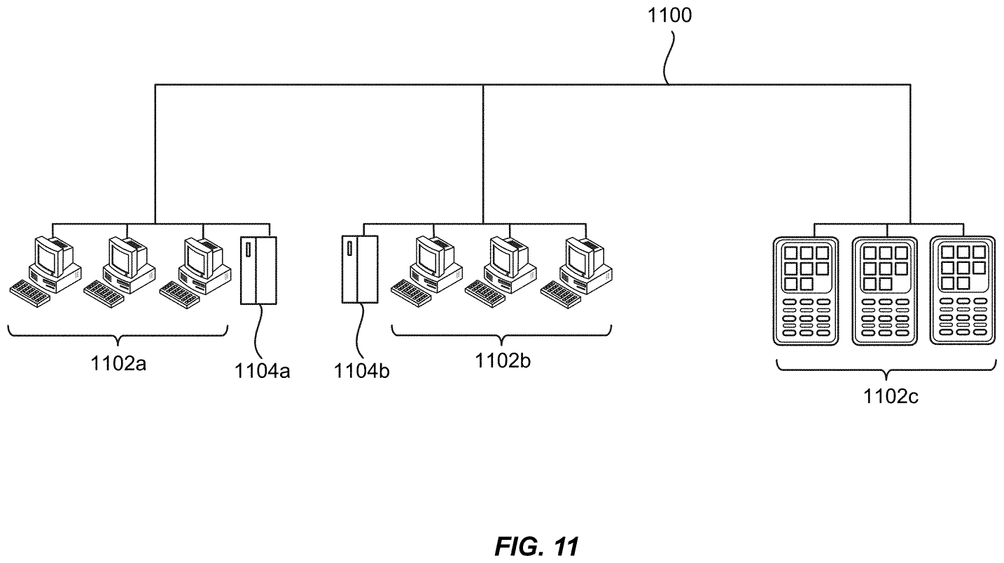

FIG. 11 is a diagram showing a network with a plurality of computer systems 1102a and 1102b, a plurality of cell phones and personal data assistants 1102c, and Network Attached Storage (NAS) 1104a and 1104b. In example embodiments, systems 1102a, 1102b, and 1102c can manage data storage and optimize data access for data stored in Network Attached Storage (NAS) 1104a and 1104b. A mathematical model can be used for the data and be evaluated using distributed parallel processing across computer systems 1102a and 1102b, and cell phone and personal data assistant systems 1102c. Computer systems 1102a and 1102b, and cell phone and personal data assistant systems 1102c can also provide parallel processing for adaptive data restructuring of the data stored in Network Attached Storage (NAS) 1104a and 1104b. FIG. 11 illustrates an example only, and a wide variety of other computer architectures and systems can be used in conjunction with the various embodiments of the present invention. For example, a blade server can be used to provide parallel processing. Processor blades can be connected through a back plane to provide parallel processing. Storage can also be connected to the back plane or as Network Attached Storage (NAS) through a separate network interface. In some instances, processors can maintain separate memory spaces and transmit data through network interfaces, back plane or other connectors for parallel processing by other processors. In some instances, some or all of the processors can use a shared virtual address memory space.

Any of the systems described herein may comprise sequence information stored on non-transitory computer readable storage media. In some instances, any of the systems described herein comprise a computer input file. In some instances, the computer input file comprises sequence information. In some instances, the computer input file comprises instructions for synthesis of a plurality of polynucleotide sequences. In some instances, the instructions are received by a computer. In some instances, the instructions are processed by the computer. In some instances, the instructions are transmitted to a material deposition device. In some instances, the non-transitory computer readable storage media is encoded with a program including instructions executable by the operating system of an optionally networked digital processing device. In some instances, a computer readable storage medium is a tangible component of a digital processing device. In some instances, a computer readable storage medium is optionally removable from a digital processing device. In some instances, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some instances, the program and instructions are permanently, substantially permanently, semi-permanently, or non-transitorily encoded on the media.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Functionalization of a Substrate Surface

A substrate was functionalized to support the attachment and synthesis of a library of polynucleotides. The substrate surface was first wet cleaned using a piranha solution comprising 90% H₂SO₄ and 10% H₂O₂ for 20 minutes. The substrate was rinsed in several beakers with deionized water, held under a deionized water gooseneck faucet for 5 min, and dried with N₂. The substrate was subsequently soaked in NH₄OH (1:100; 3 mL:300 mL) for 5 min, rinsed with DI water using a handgun, soaked in three successive beakers with deionized water for 1 min each, and then rinsed again with deionized water using the handgun. The substrate was then plasma cleaned by exposing the substrate surface to O₂. A SAMCO PC-300 instrument was used to plasma etch O₂ at 250 watts for 1 min in downstream mode.

The cleaned substrate surface was actively functionalized with a solution comprising N-(3-triethoxysilylpropyl)-4-hydroxybutyramide using a YES-1224P vapor deposition oven system with the following parameters: 0.5 to 1 torr, 60 min, 70° C., 135° C. vaporizer. The substrate surface was resist coated using a Brewer Science 200× spin coater. SPR™ 3612 photoresist was spin coated on the substrate at 2500 rpm for 40 sec. The substrate was pre-baked for 30 min at 90° C. on a Brewer hot plate. The substrate was subjected to photolithography using a Karl Suss MA6 mask aligner instrument. The substrate was exposed for 2.2 sec and developed for 1 min in MSF 26A. Remaining developer was rinsed with the handgun and the substrate soaked in water for 5 min. The substrate was baked for 30 min at 100° C. in the oven, followed by visual inspection for lithography defects using a Nikon L200. A cleaning process was used to remove residual resist using the SAMCO PC-300 instrument to O₂ plasma etch at 250 watts for 1 min.

The substrate surface was passively functionalized with a 100 μL solution of perfluorooctyltrichlorosilane mixed with 10 μL light mineral oil. The substrate was placed in a chamber, pumped for 10 min, and then the valve was closed to the pump and left to stand for 10 min. The chamber was vented to air. The substrate was resist stripped by performing two soaks for 5 min in 500 mL NMP at 70° C. with ultrasonication at maximum power (9 on Crest system). The substrate was then soaked for 5 min in 500 mL isopropanol at room temperature with ultrasonication at maximum power. The substrate was dipped in 300 mL of 200 proof ethanol and blown dry with N₂. The functionalized surface was activated to serve as a support for polynucleotide synthesis.

### Example 2: Synthesis of a 50-Mer Sequence on an Oligonucleotide Synthesis Device

A two dimensional oligonucleotide synthesis device was assembled into a flowcell, which was connected to a flowcell (Applied Biosystems (“ABI394 DNA Synthesizer”)). The two-dimensional oligonucleotide synthesis device was uniformly functionalized with N-(3-TRIETHOXYSILYLPROPYL)-4-HYDROXYBUTYRAMIDE (Gelest) was used to synthesize an exemplary polynucleotide of 50 bp (“50-mer polynucleotide”) using polynucleotide synthesis methods described herein.

The sequence of the 50-mer was as described in SEQ ID NO.: 1. 5′AGACAATCAACCATTTGGGGTGGACAGCCTTGACCTCTAGACTTCGGCAT##TTT TTTTTTT3′ (SEQ ID NO.: 1), where #denotes Thymidine-succinyl hexamide CED phosphoramidite (CLP-2244 from ChemGenes), which is a cleavable linker enabling the release of polynucleotides from the surface during deprotection.

The synthesis was done using standard DNA synthesis chemistry (coupling, capping, oxidation, and deblocking) according to the protocol in Table 3 and an ABI synthesizer.

TABLE 3
Synthesis Protocol

Table 3

| General DNA Synthesis | | Time |
|---|---|---|
| Process Name | Process Step | (sec) |
| WASH (Acetonitrile | Acetonitrile System Flush | 4 |
| Wash Flow) | Acetonitrile to Flowcell | 23 |
| | N2 System Flush | 4 |
| | Acetonitrile System Flush | 4 |
| DNA BASE ADDITION | Activator Manifold Flush | 2 |
| (Phosphoramidite + | Activator to Flowcell | 6 |
| Activator Flow) | Activator + | 6 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Incubate for 25 sec | 25 |
| WASH (Acetonitrile | Acetonitrile System Flush | 4 |
| Wash Flow) | Acetonitrile to Flowcell | 15 |
| | N2 System Flush | 4 |
| | Acetonitrile System Flush | 4 |
| DNA BASE ADDITION | Activator Manifold Flush | 2 |
| (Phosphoramidite + | Activator to Flowcell | 5 |
| Activator Flow) | Activator + | 18 |
| | Phosphoramidite to | |
| | Flowcell | |
| | Incubate for 25 sec | 25 |
| WASH (Acetonitrile | Acetonitrile System Flush | 4 |
| Wash Flow) | Acetonitrile to Flowcell | 15 |
| | N2 System Flush | 4 |
| | Acetonitrile System Flush | 4 |
| CAPPING (Cap A + B, | Cap A + B to Flowcell | 15 |
| 1:1, Flow) | | |
| WASH (Acetonitrile | Acetonitrile System Flush | 4 |
| Wash Flow) | Acetonitrile to Flowcell | 15 |
| | Acetonitrile System Flush | 4 |
| OXIDATION | Oxidizer to Flowcell | 18 |
| (Oxidizer Flow) | | |
| WASH (Acetonitrile | Acetonitrile System Flush | 4 |
| Wash Flow) | N2 System Flush | 4 |
| | Acetonitrile System Flush | 4 |
| | Acetonitrile to Flowcell | 15 |
| | Acetonitrile System Flush | 4 |
| | Acetonitrile to Flowcell | 15 |
| | N2 System Flush | 4 |
| | Acetonitrile System Flush | 4 |
| | Acetonitrile to Flowcell | 23 |
| | N2 System Flush | 4 |
| | Acetonitrile System Flush | 4 |
| DEBLOCKING | Deblock to Flowcell | 36 |
| (DeblockFlow) | | |
| WASH (Acetonitrile | Acetonitrile System Flush | 4 |
| Wash Flow) | N2 System Flush | 4 |
| | Acetonitrile System Flush | 4 |
| | Acetonitrile to Flowcell | 18 |
| | N2 System Flush | 4.13 |
| | Acetonitrile System Flush | 4.13 |
| | Acetonitrile to Flowcell | 15 |

The phosphoramidite/activator combination was delivered similar to the delivery of bulk reagents through the flowcell. No drying steps were performed as the environment stays “wet” with reagent the entire time.

The flow restrictor was removed from the ABI394 DNA Synthesizer to enable faster flow. Without flow restrictor, flow rates for amidites (0.1M in ACN), Activator, (0.25M Benzoylthiotetrazole (“BTT”; 30-3070-xx from GlenResearch) in ACN), and Ox (0.02M 12 in 20% pyridine, 10% water, and 70% THF) were roughly ˜100 uL/sec, for acetonitrile (“ACN”) and capping reagents (1:1 mix of CapA and CapB, wherein CapA is acetic anhydride in THF/Pyridine and CapB is 16% 1-methylimidizole in THF), roughly ˜200 uL/sec, and for Deblock (3% dichloroacetic acid in toluene), roughly ˜300 uL/sec (compared to −50 uL/sec for all reagents with flow restrictor). The time to completely push out Oxidizer was observed, the timing for chemical flow times was adjusted accordingly and an extra ACN wash was introduced between different chemicals. After polynucleotide synthesis, the chip was deprotected in gaseous ammonia overnight at 75 psi. Five drops of water were applied to the surface to recover polynucleotides. The recovered polynucleotides were then analyzed on a BioAnalyzer small RNA chip (data not shown).

### Example 3: Synthesis of a 100-Mer Sequence on an Oligonucleotide Synthesis Device

The same process as described in Example 2 for the synthesis of the 50-mer sequence was used for the synthesis of a 100-mer polynucleotide (“100-mer polynucleotide”; 5′ CGGGATCCTTATCGTCATCGTCGTACAGATCCCGACCCATTTGCTGTCCACCAGT CATGCTAGCCATACCATGATGATGATGATGATGAGAACCCCGCAT##TTTTTTTTT T3′, where #denotes Thymidine-succinyl hexamide CED phosphoramidite (CLP-2244 from ChemGenes); SEQ ID NO.: 2) on two different silicon chips, the first one uniformly functionalized with N-(3-TRIETHOXYSILYLPROPYL)-4-HYDROXYBUTYRAMIDE and the second one functionalized with 5/95 mix of 11-acetoxyundecyltriethoxysilane and n-decyltriethoxysilane, and the polynucleotides extracted from the surface were analyzed on a BioAnalyzer instrument (data not shown).

All ten samples from the two chips were further PCR amplified using a forward (5′ATGCGGGGTTCTCATCATC3′; SEQ ID NO.: 3) and a reverse (5′CGGGATCCTTATCGTCATCG3′; SEQ ID NO.: 4) primer in a 50 uL PCR mix (25 uL NEB Q5 mastermix, 2.5 uL 10 uM Forward primer, 2.5 uL 10 uM Reverse primer, 1 uL polynucleotide extracted from the surface, and water up to 50 uL) using the following thermal cycling program:

98° C., 30 sec

98° C., 10 sec; 63° C., 10 sec; 72° C., 10 sec; repeat 12 cycles

72° C., 2 min

The PCR products were also run on a BioAnalyzer (data not shown), demonstrating sharp peaks at the 100-mer position. Next, the PCR amplified samples were cloned, and Sanger sequenced. Table 4 summarizes the results from the Sanger sequencing for samples taken from spots 1-5 from chip 1 and for samples taken from spots 6-10 from chip 2.

TABLE 4
Sequencing Results

| | | Cycle |
|---|---|---|
| Spot | Error rate | efficiency |

| | | | |
|---|---|---|---|
| 1 | 1/763 | bp | 99.87% |
| 2 | 1/824 | bp | 99.88% |
| 3 | 1/780 | bp | 99.87% |
| 4 | 1/429 | bp | 99.77% |
| 5 | 1/1525 | bp | 99.93% |
| 6 | 1/1615 | bp | 99.94% |
| 7 | 1/531 | bp | 99.81% |
| 8 | 1/1769 | bp | 99.94% |
| 9 | 1/854 | bp | 99.88% |
| 10 | 1/1451 | bp | 99.93% |

Thus, the high quality and uniformity of the synthesized polynucleotides were repeated on two chips with different surface chemistries. Overall, 89%, corresponding to 233 out of 262 of the 100-mers that were sequenced were perfect sequences with no errors. Table 5 summarizes error characteristics for the sequences obtained from the polynucleotides samples from spots 1-10.

TABLE 5
Error Characteristics

| Sample ID/Spot no. | OSA_0046/1 | OSA_0047/2 | OSA_0048/3 | OSA_0049/4 | OSA_0050/5 |
|---|---|---|---|---|---|
| Total Sequences | 32 | 32 | 32 | 32 | 32 |
| Sequencing Quality | 25 of 28 | 27 of 27 | 26 of 30 | 21 of 23 | 25 of 26 |
| Oligo Quality | 23 of 25 | 25 of 27 | 22 of 26 | 18 of 21 | 24 of 25 |
| ROI Match Count | 2500 | 2698 | 2561 | 2122 | 2499 |
| ROI Mutation | 2 | 2 | 1 | 3 | 1 |
| ROI Multi Base Deletion | 0 | 0 | 0 | 0 | 0 |
| ROI Small Insertion | 1 | 0 | 0 | 0 | 0 |
| ROI Single Base Deletion | 0 | 0 | 0 | 0 | 0 |
| Large Deletion Count | 0 | 0 | 1 | 0 | 0 |
| Mutation: G > A | 2 | 2 | 1 | 2 | 1 |
| Mutation: T > C | 0 | 0 | 0 | 1 | 0 |
| ROI Error Count | 3 | 2 | 2 | 3 | 1 |
| ROI Error Rate | Err: ~1 in 834 | Err: ~1 in 1350 | Err: ~1 in 1282 | Err: ~1 in 708 | Err: ~1 in 2500 |
| ROI Minus Primer Error Rate | MP Err: ~1 in 763 | MP Err: ~1 in 824 | MP Err: ~1 in 780 | MP Err: ~1 in 429 | MP Err: ~1 in 1525 |
| Sample ID/Spot no. | OSA_0051/6 | OSA_0052/7 | OSA_0053/8 | OSA_0054/9 | OSA_0055/10 |
| Total Sequences | 32 | 32 | 32 | 32 | 32 |
| Sequencing Quality | 29 of 30 | 27 of 31 | 29 of 31 | 28 of 29 | 25 of 28 |
| Oligo Quality | 25 of 29 | 22 of 27 | 28 of 29 | 26 of 28 | 20 of 25 |
| ROI Match Count | 2666 | 2625 | 2899 | 2798 | 2348 |
| ROI Mutation | 0 | 2 | 1 | 2 | 1 |
| ROI Multi Base Deletion | 0 | 0 | 0 | 0 | 0 |
| ROI Small Insertion | 0 | 0 | 0 | 0 | 0 |
| ROI Single Base Deletion | 0 | 0 | 0 | 0 | 0 |
| Large Deletion Count | 1 | 1 | 0 | 0 | 0 |
| Mutation: G > A | 0 | 2 | 1 | 2 | 1 |
| Mutation: T > C | 0 | 0 | 0 | 0 | 0 |
| ROI Error Count | 1 | 3 | 1 | 2 | 1 |
| ROI Error Rate | Err: ~1 in 2667 | Err: ~1 in 876 | Err: ~1 in 2900 | Err: ~1 in 1400 | Err: ~1 in 2349 |
| ROI Minus Primer Error Rate | MP Err: ~1 in 1615 | MP Err: ~1 in 531 | MP Err: ~1 in 1769 | MP Err: ~1 in 854 | MP Err: ~1 in 1451 |

### Example 4. Exemplary Formulations for Enzymatic Based Assembly

Various reaction conditions are seen in Tables 6-14. The reagents are added in various orders. Alternatively, the reagents are added in step wise fashion, for example, reagents are added in order listed as in Table 14.

TABLE 6
Reaction Conditions 1

| | Final | |
|---|---|---|
| Reagent | Concentration | |
| Vector | 4 | nM |
| Gene Fragment 1 | 4 | nM |
| dNTP | .2 | mM |
| 10X Ampligase buffer | 1X | |
| ExoIII | 10 | U |
| Phusion | 0.2 | U |
| Ampligase | 1 | U |
| Fen1 | 3.2 | U |

| | |
|---|---|
| Water | Remaining water |
| | up to 10 uL |

TABLE 7
Reaction Conditions 2

| | Final | |
|---|---|---|
| Reagent | Concentration | |
| Vector | 4 | nM |
| Gene Fragment | 4 | nM |
| dNTP | 0.2 | mM |
| 10X Ampligase buffer | 1X | |
| ExoIII | 1 | U |
| Phusion | 0.2 U or 0.1 U | |
| Ampligase | 1 | U |
| Fen1 | 0.32 | U |

| | |
|---|---|
| Water | Remaining water |
| | up to 10 uL |

TABLE 8
Enzyme Concentrations

| | Reaction Condition | |
|---|---|---|
| 1 | 0.32 | U Fen1 |
| | 1 | U ExoIII |
| | 0.2 | U Phusion |
| | 1 | U Ampligase |
| 2 | 0.32 | U Fen1 |
| | 1 | U ExoIII |
| | 0.1 | U Phusion |
| | 0.5 | U Ampligase |
| 3 | 0.32 | U Fen1 |
| | 1 | U ExoIII |
| | 0.1 | U Phusion |
| | 1.0 | U Ampligase |
| 4 | 0.32 | U Fen1 |
| | 1 | U ExoIII |
| | 0.05 | U Phusion |
| | 1.0 | U Ampligase |
| 5 | 0.32 | U Fen1 |
| | 1.5 | U ExoIII |
| | 0.2 | U Phusion |
| | 1.0 | U Ampligase |
| 6 | 4.8 | U Fen1 |
| | 1.0 | U ExoIII |
| | 0.2 | U Phusion |
| | 1.0 | U Ampligase |
| 7 | 0.32 | U Fen1 |
| | 0.5 | U ExoIII |
| | 0.05 | U Phusion |
| | 1.0 | U Ampligase |
| 8 | 0.32 | U Fen1 |
| | 1.0 | U ExoIII |
| | 0.1 | U Phusion |
| | 0.1 | U Ampligase |
| 9 | 0.32 | U Fen1 |
| | 1.0 | U ExoIII |
| | 0.1 | U Phusion |
| | 0.25 | U Ampligase |
| 10 | 0.32 | U Fen1 |
| | 1.0 | U ExoIII |
| | 0.2 | U Phusion |
| | 0.5 | U Ampligase |
| 11 | 0.32 | U Fen1 |
| | 1.0 | U ExoIII |
| | 0.2 | U Phusion |
| | 0.25 | U Ampligase |
| 12 | 0.32 | U Fen1 |
| | 0.5 | U ExoIII |
| | 0.1 | U Phusion |
| | 1.0 | U Ampligase |
| 13 | 3.2 | U Fen1 |
| | 1.0 | U ExoIII |
| | 0.2 | U Phusion |
| | 1.0 | U Ampligase |
| 14 | 0.32 | U Fen1 |
| | 0.5 | U ExoIII |
| | 0.2 | U Phusion |
| | 1.0 | U Ampligase |
| 15 | 0.32 | U Fen1 |
| | 1.5 | U ExoIII |
| | 0.1 | U Phusion |
| | 1.0 | U Ampligase |
| 16 | 0.32 | U Fen1 |
| | 1.5 | U ExoIII |
| | 0.05 | U Phusion |
| | 1.0 | U Ampligase |
| 17 | 3.2 | U Fen1 |
| | 0.5 | U ExoIII |
| | 0.2 | U Phusion |
| | 0.5 | U Ampligase |
| 18 | 3.2 | U Fen1 |
| | 1.0 | U ExoIII |
| | 0.2 | U Phusion |
| | 0.5 | U Ampligase |
| 19 | 3.2 | U Fen1 |
| | 1.0 | U ExoIII |
| | 0.2 | U Phusion |
| | 0 | U Ampligase |
| 20 | 4.8 | U Fen1 |
| | 0.5 | U ExoIII |
| | 0.2 | U Phusion |
| | 1.0 | U Ampligase |
| 21 | 0.32 | U Fen1 |
| | 1.5 | U ExoIII |
| | 0.5 | U Phusion |
| | 1.0 | U Ampligase |
| 22 | 3.2 | U Fen1 |
| | 0.5 | U ExoIII |
| | 0.2 | U Phusion |
| | 1.0 | U Ampligase |
| 23 | 0.32 | U Fen1 |
| | 1.0 | U ExoIII |
| | 0.2 | U Phusion |
| | 0.1 | U Ampligase |
| 24 | 0.32 | U Fen1 |
| | 0.5 | U ExoIII |
| | 0.5 | U Phusion |
| | 1.0 | U Ampligase |
| 25 | 0.32 | U Fen1 |
| | 1.0 | U ExoIII |
| | 0.5 | U Phusion |
| | 1.0 | U Ampligase |
| 26 | 3.2 | U Fen1 |
| | 10.0 | U ExoIII |
| | 0.2 | U Phusion |
| | 1.0 | U Ampligase |
| 27 | 3.2 | U Fen1 |
| | 5.0 | U ExoIII |
| | 0.2 | U Phusion |
| | 1.0 | U Ampligase |

TABLE 9
Method 1 Reaction Concentrations

| | 5 uL | Final | |
|---|---|---|---|
| Reagent | reaction | Concentration | |
| dNTP (10 mM) | 0.1 | .2 | mM |
| 10x Ampligase | 0.5 | 1X | |
| buffer | | | |
| ExoIII (100 U/uL) | 0.005 | 0.1 | U/uL |
| Phusion (2 U/uL) | 0.05 | 0.02 | U/uL |
| Ampligase (5 U/uL) | 0.1 | 0.1 | U/uL |
| Fen1 (32 U/uL) | 0.005 | 0.032 | U/uL |
| Vector DNA | | 20 | fmol |
| Insert DNA | | 40 | fmol/Insert |
| Water * | To 5 uL | | |

TABLE 10
Method 2 Reaction Concentrations

| | 5 uL | Final | |
|---|---|---|---|
| Reagent | reaction | Concentration | |
| dNTP (10 mM) | 0.1 | .2 | mM |
| 10x Ampligase | 0.5 | 1x | |
| buffer | | | |
| ExoIII 100 U/uL | 0.08 | 1.6 | U/uL |
| Phusion 2 U/uL | 0.05 | 0.02 | U/uL |
| Ampligase 5 U/uL | 0.1 | 0.1 | U/uL |
| Fen1 32 U/uL | 0.005 | 0.032 | U/uL |
| Vector DNA | | 20 | fmol |
| Insert DNA | | 40 | fmol/Insert |
| Water * | To 5 uL | | |

TABLE 11
Method 3 Reaction Conditions

| | 5 uL | Final | |
|---|---|---|---|
| Master Mix | reaction | Concentration | |
| dNTP | 0.1 | .4 | mM |
| 10x Ampligase buffer | 0.5 | 2x | |
| (Epicenter) | | | |
| ExoIII 100 U/uL | 0.08 | 1.6 | U/uL |
| (NEB) | | | |
| Phusion 2 | 0.005 | 0.002 | U/uL |
| U/uL (NEB) | | | |
| Ampligase 5 U/uL | 0.1 | 0.1 | U/uL |
| (Epicenter) | | | |
| Fen1 32 U/uL (NEB) | 0.005 | 0.032 | U/uL |
| Water * | 1.695 | | |

TABLE 12
Method 4 Reaction Conditions

| | | Final | |
|---|---|---|---|
| | 50 ul | Concentration | |
| Master Mix | reaction | (for 2x MM) | |
| ExoII 100 U/uL | 0.05 | 0.2 | U/uL |
| Phusion 2 U/uL | 0.05 | 0.004 | U/uL |
| Fen1 32 U/uL | 0.05 | 0.064 | U/uL |
| dNTP | 1 | .4 | mM |
| Ampligase | 1 | 0.2 | U/uL |
| 5 U/uL | | | |

| | | |
|---|---|---|
| 10x Ampligase | 5 | 2x |
| buffer | | |
| Water * | | |

TABLE 13
Method 5 Reaction Conditions

| Stepwise | | Final | | Volume for |
|---|---|---|---|---|
| addition | | Concentration | | 250 uL |
| step | Component | (for 2x MM) | | Master Mix |
| 1 | Water | | | 179.75 |
| 2 | 10x Taq HiFi | 2x | | 50 |
| | DNA ligase | | | |
| | buffer | | | |
| 3 | dNTP | .4 | mM | 10 |
| 4 | ExoIII | 3.2 | U/uL | 8 |
| | 100 U/uL | | | |
| 5 | Phusion 2 U/uL | 0.004 | U/uL | 0.5 |
| 6 | Taq DNA | 0.2 | U/uL | 1.25 |
| | ligase | | | |
| | 40 U/uL | | | |
| 7 | Fen1 32 U/uL | 0.064 | U/uL | 0.5 |

TABLE 14
Reaction Conditions

| | Final | |
|---|---|---|
| | Concentration | |
| Component | (for 2x MM) | |
| Water | | |
| 10x Taq HIFI | 0.5-5x | |
| DNA ligase | | |
| buffer | | |
| dNTP | 0.1-1.0 | mM |
| ExoIII | 0.8-8 | U/uL |
| 100 U/uL | | |
| Phusion 2 U/uL | 0.001-0.01 | U/uL |
| Taq DNA | 0.05-5.0 | U/uL |
| ligase | | |
| 40 U/uL | | |
| Fen1 32 U/uL | 0.01-0.1 | U/uL |

### Example 5. Enzymatic Mediated Nucleic Acid Assembly

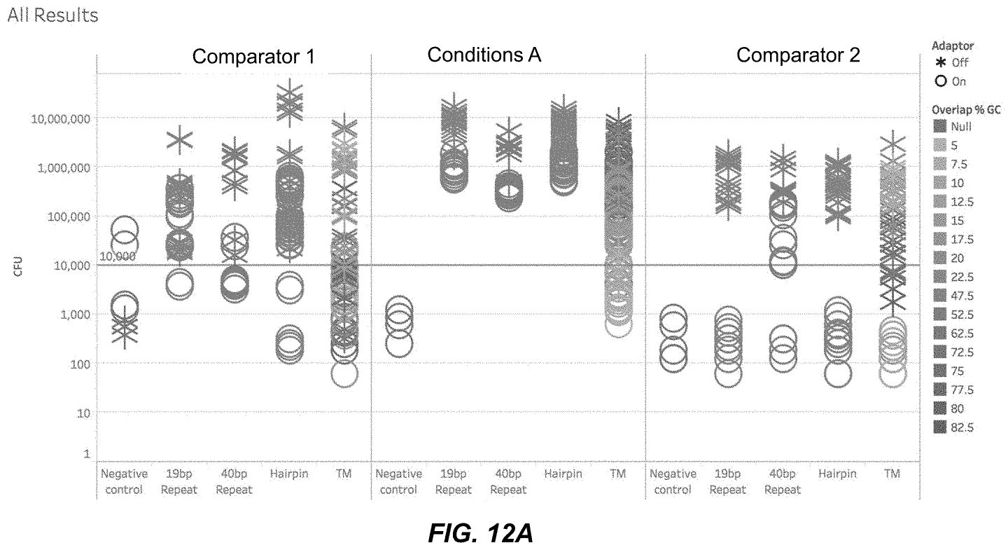

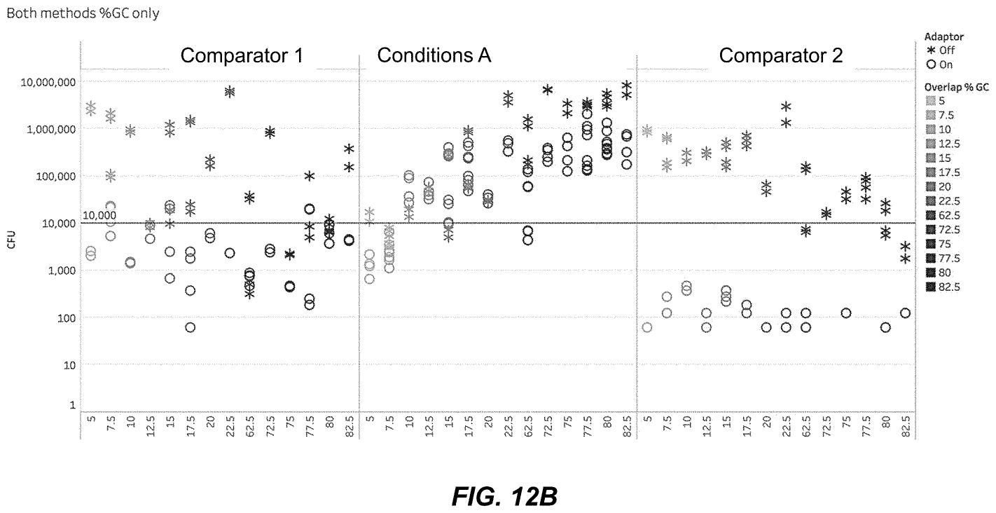

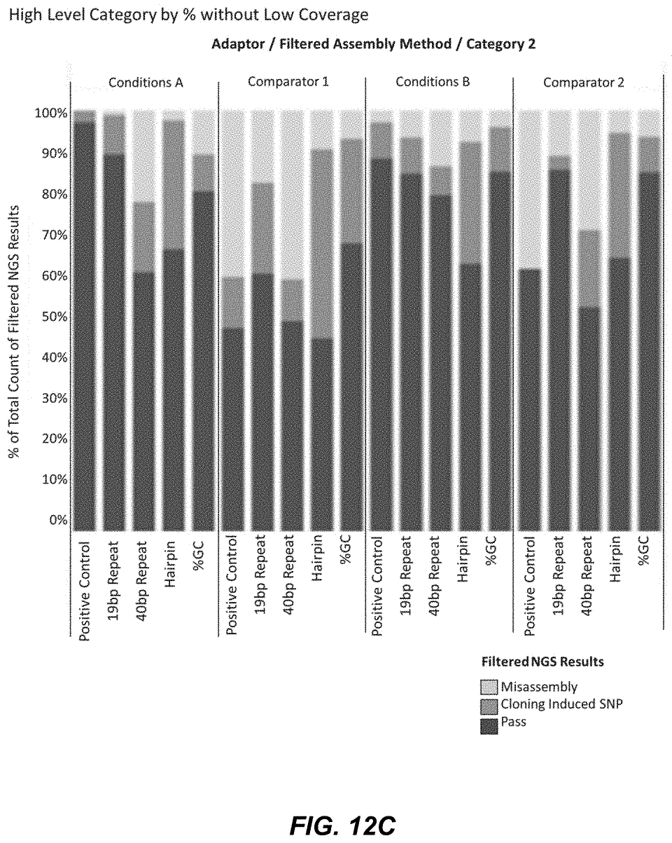

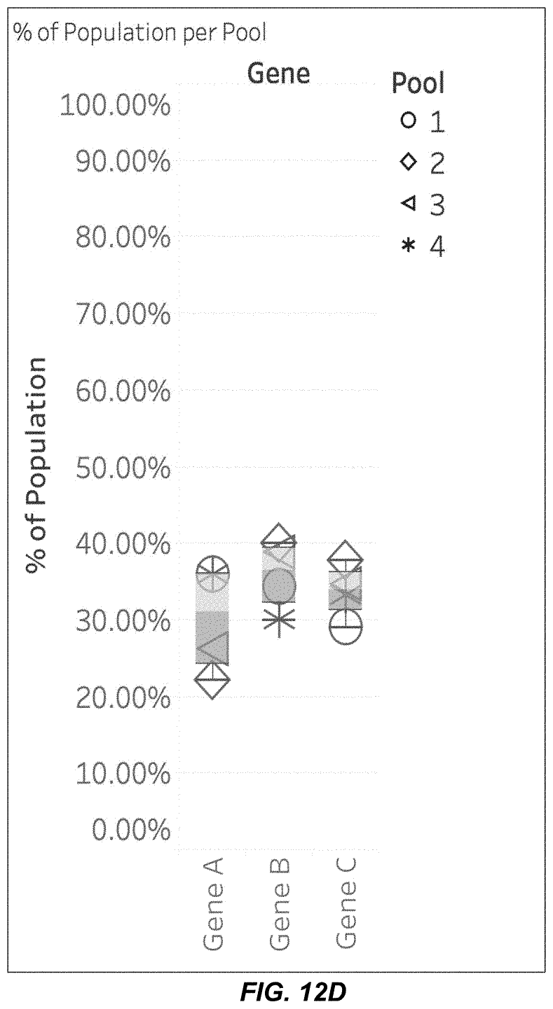

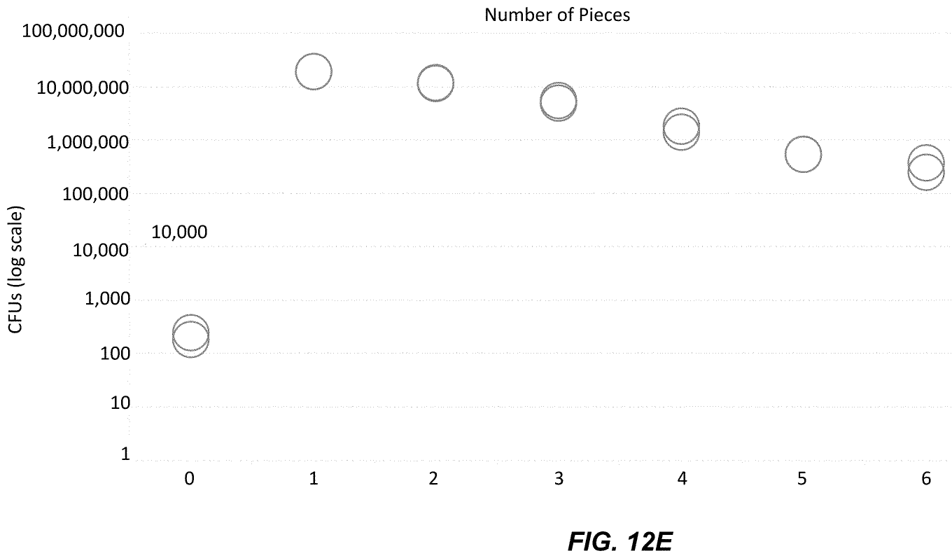

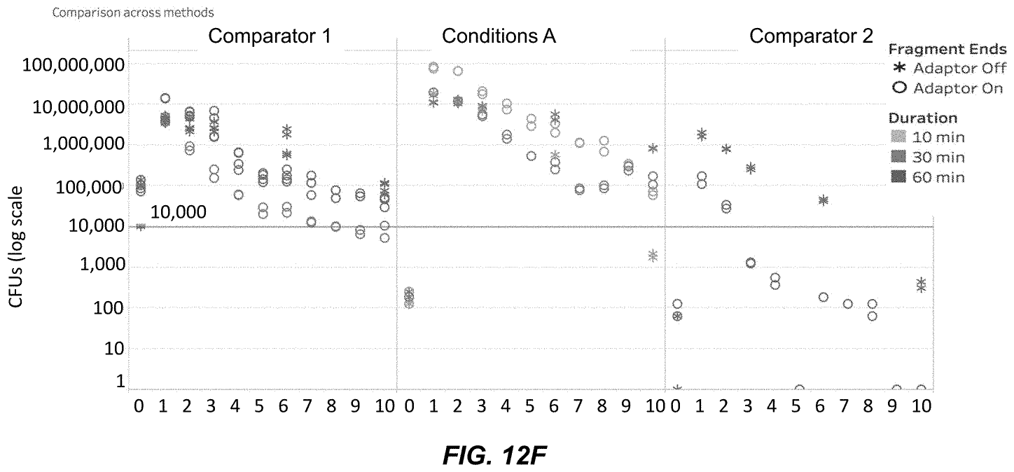

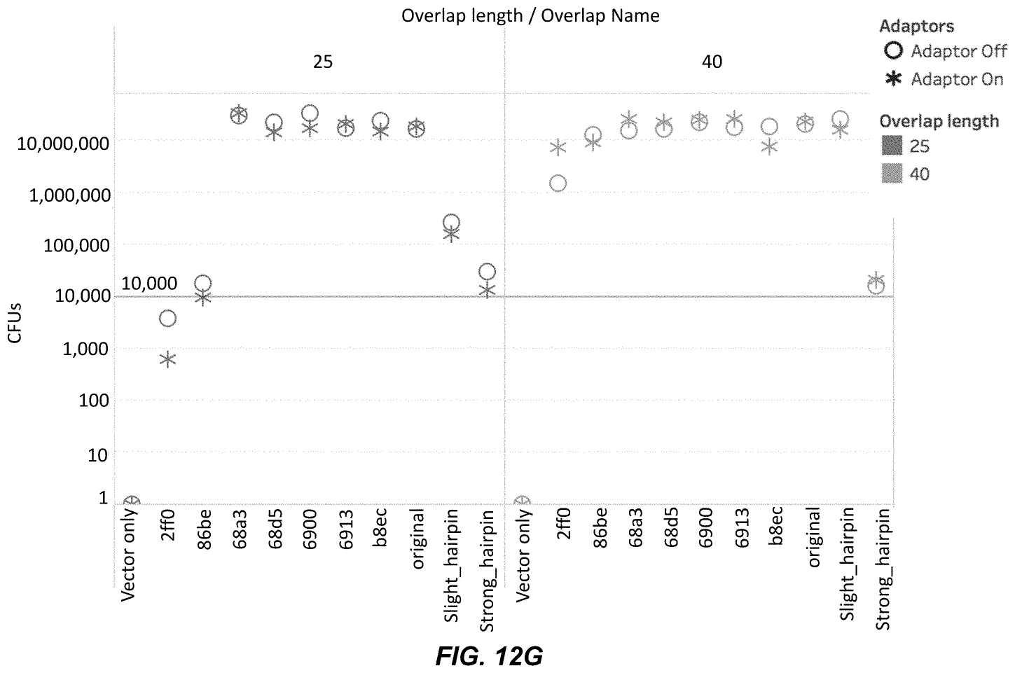

Enzymatic mediated nucleic acid (guided assembly) using reaction conditions as described in Example 4 was performed (“Conditions A”). Enzymatic mediated nucleic acid assembly resulted in high colony forming units (CFUs) even in the presence of direct repeats flanking homology sequences and secondary structures (FIG. 12A). Furthermore, CFUs from the enzymatic mediated nucleic acid assembly were tightly distributed, demonstrating robust reaction conditions. A/T rich overlap homology sequences contained less than 10% GC as seen in FIG. 12B. As compared to Comparator 1 and Comparator 2 assembly (alternative exonuclease/ligase-based assembly methods), enzymatic mediated nucleic acid assembly was significantly more efficient with homology regions over 72.5% GC. Accuracy was also measured by NGS sequencing 8-12 clones. There was no significant impact to enzymatic mediated nucleic acid assembly accuracy with the extreme GC %, hairpins or direct repeats; average enzymatic mediated nucleic acid assembly pass rates ranged from 56% to 88% regardless of the presence or absence of universal adapter sequences. Comparator 1 and Comparator 2 assembly performed more poorly. Comparator 2 assembly reactions had pass rates ranging from 41% to 56% and Comparator 1 had pass rates ranging 53% to 75% (FIG. 12C).

Assembly specificity and sequence bias were evaluated through multiplexed gene assembly (FIG. 12D). Assembly of three different genes (Gene A, Gene B, Gene C), composed of nine dsDNA input fragments with universal adapters were assembled in a single reaction. Homology sequence similarities ranged between 28-60%. In parallel independent reactions (N=4), the nine input fragments were subjected to enzymatic mediated nucleic acid assembly to form three genes. All constructs shared 5′ and 3′ primer sites, PCR amplified to enrich for the full length gene, cloned into a plasmid using the enzymatic mediated nucleic acid assembly and transformed into E. coli. Ninety six colonies from each reaction pool were isolated for Sanger sequenced and the final constructs sequenced. All sequencing reads indicated full length constructs for the desired genes and did not show evidence of universal adapter sequences, chimeric gene sequences, or misassemblies. As seen in FIG. 12D, a tight distribution of each gene sequence around the expected average of 33% was observed, again demonstrating accuracy and specificity of enzymatic mediated nucleic acid assembly without sequence bias. Larger fragments were also successfully assembled. Using the enzymatic assembly method, six DNA fragments were assembled at once using an enzymatic reaction, with a high number of colony forming units obtained (FIG. 12E). Conditions A resulted in a higher number of CFUs for assembly of larger fragments (up to 10) than comparator 1 or comparator 2 conditions as shown in FIG. 12F. Additional design elements such as optimal homology lengths between fragments was tested (FIG. 12G).

### Example 6. 400 Base Pair Multiplex Gene Assembly

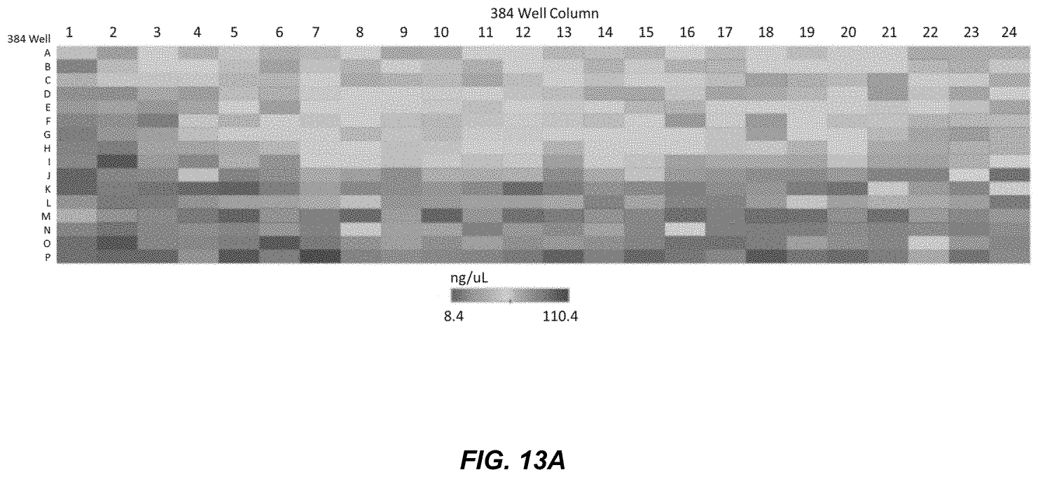

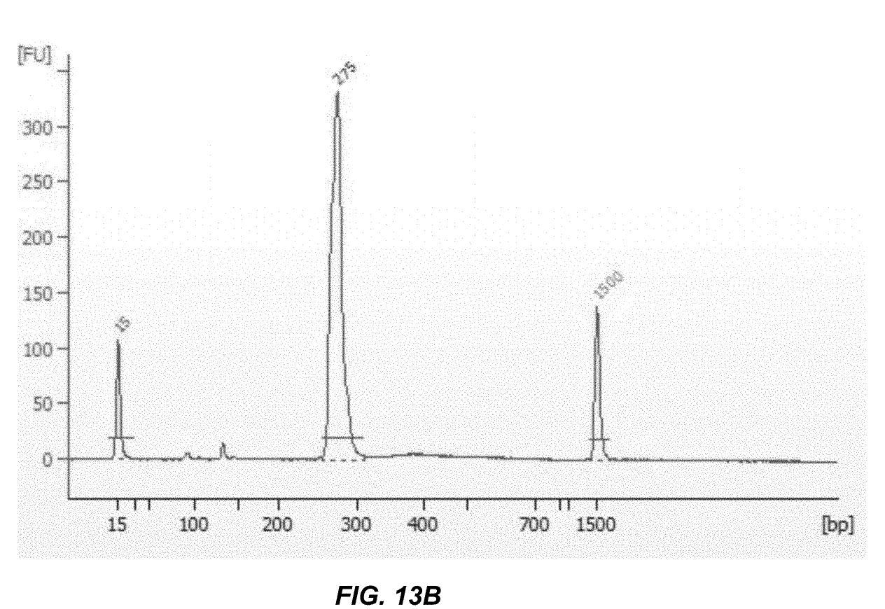

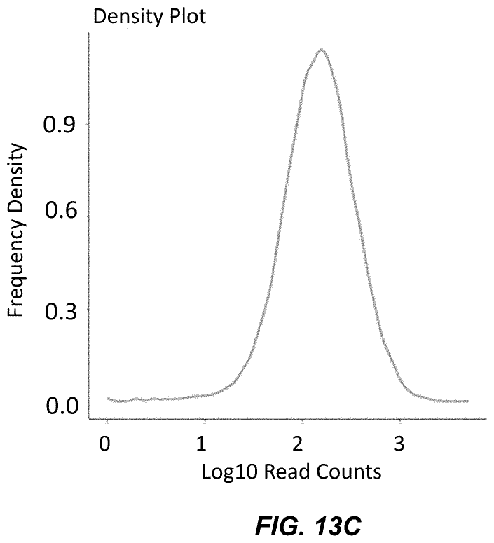

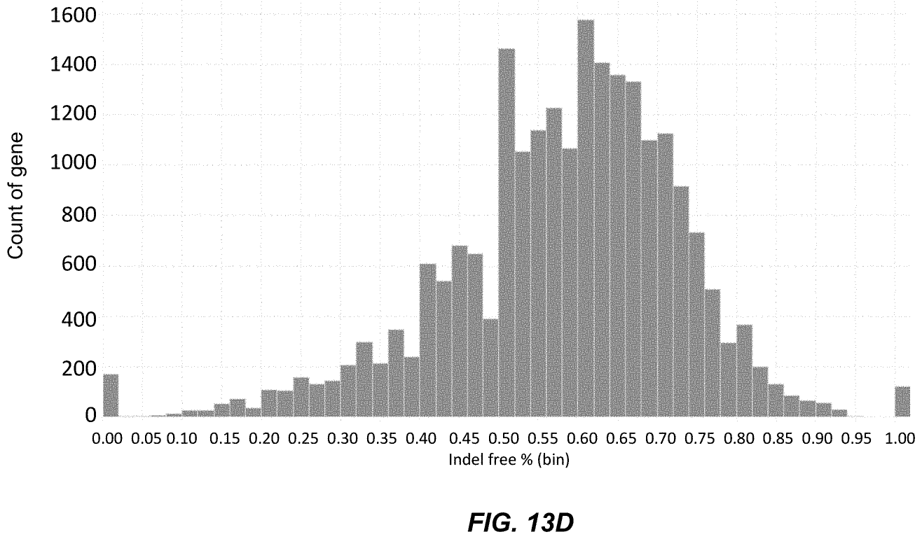

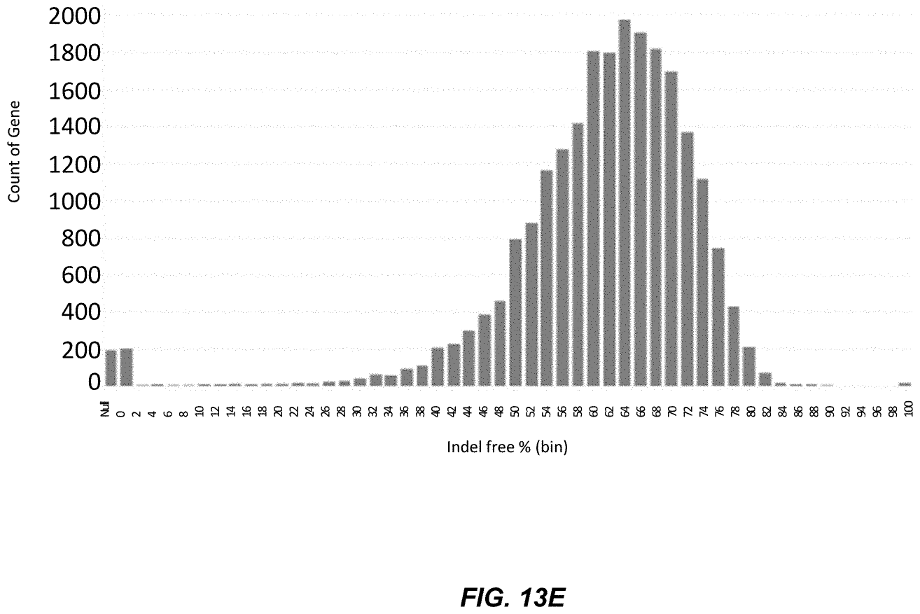

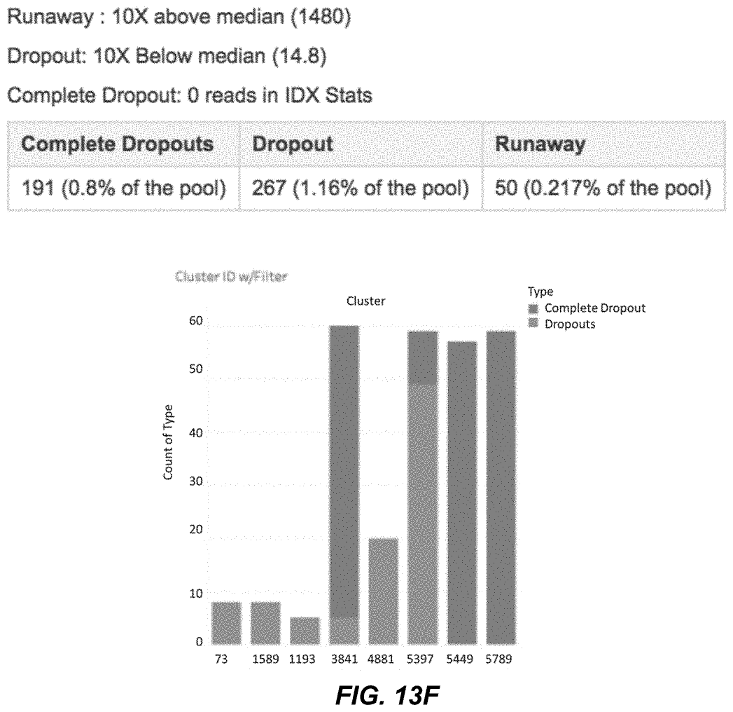

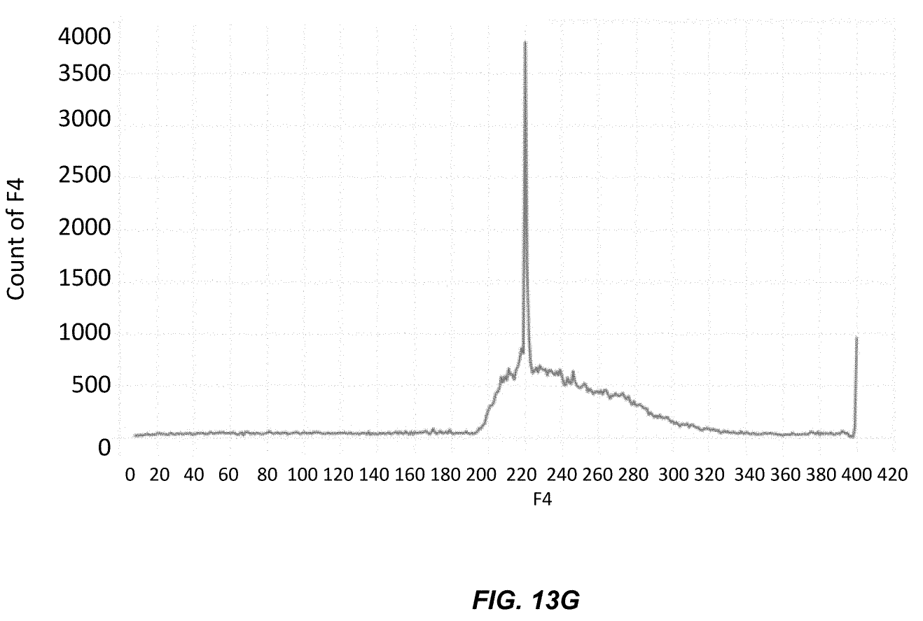

Multiplexed assembly of 60 genes/cluster was performed using 270mer nucleic acids comprising Uni9 universal primers. Data from assembly of 23,000 genes is shown in FIGS. 13A-13G. FIG. 13A shows relative concentrations of DNA following PCR using universal primers. FIG. 13B shows a plot from a BioAnalyzer reading. FIGS. 13C-13E shows next generation sequencing (NGS) results, specifically a density plot using 140× coverage (FIG. 13C) and distribution of percentage of insertion/deletion free genes (FIGS. 13D-13E). FIG. 13F shows percentage of complete dropout, dropout, and runaway. FIG. 13G shows a graph of soft clipping/chimeric reads. About 1% of the population of nucleic acids comprises chimeric gene fragments. Results are also seen in Tables 15-16 below.

TABLE 15
QC Metrics

| | | | | | Average % |
|---|---|---|---|---|---|
| | Complete | | | % Genes | Indel-Free |
| | Dropouts | Dropouts | Runaways | with at least | Rate for a |
| QC | (missing | (Outside | (Outside | 1 indel-Free | Gene in |
| Metric | sequence) | 10-fold range) | 10-fold range) | Sequence | Pool |
| Population 1 | 1.09% | 1.12% | 0.23% | 98.32% | 62.3% |
| Population 2 | 0.3% | 1.38% | 0A % | 98.12% | 54.12% |
| Population 3 | 0.29% | 1.38% | 0A % | 98.09% | 52.56% |
| Population 4 | 0.19% | 1.3% | 0.44% | 98.44% | 51.94% |
| Population 5 | 0.2% | 1.68% | 0.3% | 98.5% | 59% |
| Population 6 | 0.18% | 1.05% | 0.33% | 98.73% | 52.47% |
| Population 7 | 0.2% | 1.78% | 0.26% | 98.5% | 60% |
| Population 8 | | | | | |
| Population 9 | | | | | |
| Population 10 | 0.2% | 1.38% | 0.23% | 98.64% | 60.5% |
| Population 11 | 0.27% | 1.55% | 0.20% | 98.57% | 58.8% |

TABLE 16
QC Metrics Percentile

| Uniformity | 90^{th}/10^{th} | 95^{th}/5^{th} |
|---|---|---|
| Table | Percentile | Percentile |
| Population 1 | 9.08 | 18.12 |
| Population 2 | 11.8 | 23.3 |
| Population 3 | 10.7 | 23.6 |
| Population 4 | 10.8 | 23.1 |
| Population 5 | 11.9 | 27.17 |
| Population 6 | 9.6 | 19.8 |
| Population 7 | 10.89 | 22.17 |
| Population 8 | | |
| Population 9 | | |
| Population 10 | 10.64 | 22.71 |
| Population 11 | 11.69 | 23 |

### Example 7. Combinatorial Assembly of Variants

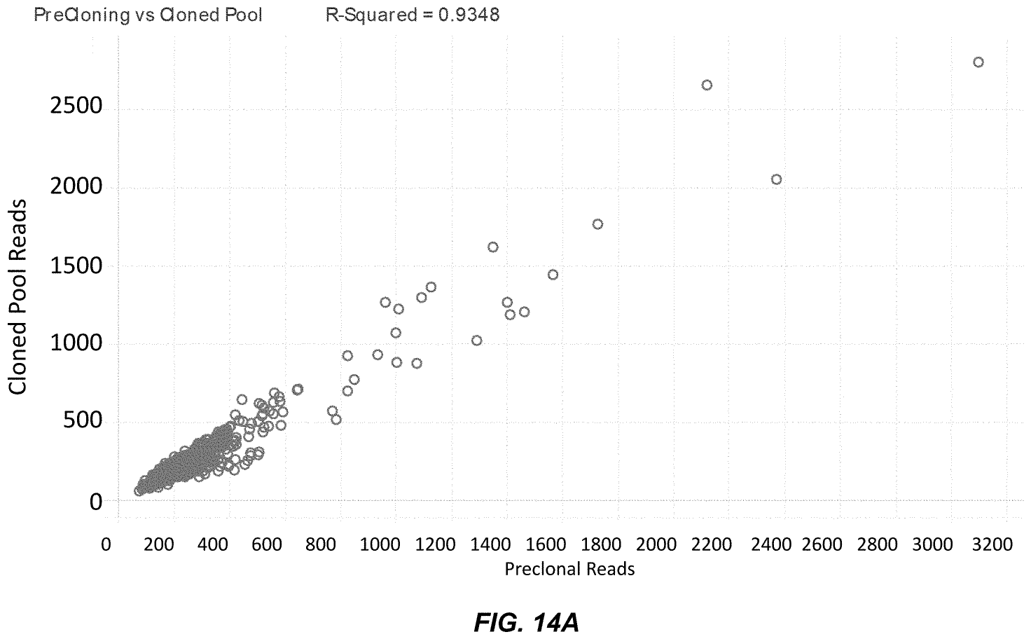

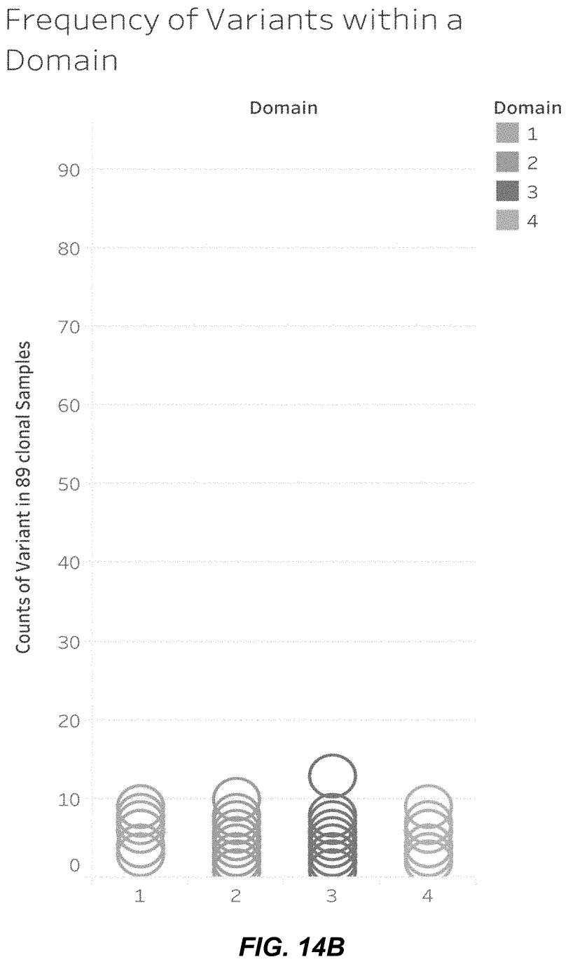

Combinatorial assembly of variants was performed using methods as described herein. Four input populations were assembled. Assembly resulted in about 150,000 variants and uniformity of full length sequences before and after cloning (FIG. 14A) as well as uniform variant frequency (FIG. 14B). After assembly, products were PCR amplified to enrich for the full length gene then cloned into a plasmid and transformed into E. coli. 96 colonies from each reaction pool were isolated for Sanger sequencing. All sequencing reads indicated full length constructs for the desired genes. There was no observation of internal universal adapter sequences, chimeric gene sequences, or misassemblies.

### Example 8. Scalable Assembly Using Enzymatic Mediated Nucleic Acid Assembly

Enzymatic mediated nucleic acid assembly was performed using the Labcyte Echo® 525 Liquid Handler, to generate actionable DNA constructs on a large scale. In a single pot reaction, miniaturized enzymatic mediated nucleic acid assembly reactions were used to assemble two linear dsDNA fragments into a vector enabling fluorescent protein expression under a wild-type and variant p70 promoter. p70 promoter tuning was assessed by driving expression of the fluorescent protein mCherry under a wild-type (WT) promoter and test synthesized p70 variants differentially driving GFP expression in the same construct. By normalizing the GFP to mCherry signal, the various mutated p70 promoter strengths were approximated. As a result of multiplexing DNA assembly and myTXTL® protein synthesis, optimal protein production conditions were ascertained within the miniaturized reactions.

### Example 9. Immunoglobulin Sequence Assembly

This example illustrates a de novo synthesis method for immunoglobulin sequence assembly.

A first leader sequence, a first variable region, and a first CDR segment are synthesized and then subject to polymerase chain assembly (PCA) to generate a first plurality of gene fragments. A second leader sequence, a second variable region, and a second CDR segment are synthesized and then subject to assembly PCR or PCA to generate a second plurality of gene fragments. A third plurality of gene fragments comprising a second constant region followed by a self-cleaving peptide and a fourth plurality of gene fragments comprising a variable constant segment are synthesized. The third plurality of gene fragments and the fourth plurality of gene fragments are added to the first plurality of gene fragments and the second plurality of gene fragments followed by PCR. An error correction reaction may optionally be performed. The resulting construct is pooled, cloned, and subject to next generation sequencing.

### Example 10. Multiplex Immunoglobulin Sequence Assembly

This example illustrates a de novo synthesis method for multiplex immunoglobulin sequence assembly.

Gene fragments are synthesized comprising variants of a first variable region and amplified with gene fragments comprising a 40 base pair (bp) region complementary to the first variable region and a first CDR and J segment to generate a first plurality of gene fragments. Gene fragments are synthesized comprising variants of a second variable region and amplified with gene fragments comprising a second CDR and J segment to generate a second plurality of gene fragments. A third plurality of gene fragments is synthesized comprising a constant region, a self-cleaving peptide sequence, a first leader sequence, and a 40 base pair (bp) region complementary to the second variable region and a second CDR and J segment. The self-cleaving peptide sequence is P2A.

The first plurality of gene fragments, the second plurality of gene fragments, and the third plurality of gene fragments are assembled using an enzymatic based assembly method, PCR purified, and pooled. All non-assembled fragments are purified away. The final construct is then cloned into a vector.

### Example 11. Paired Variant Assembly Using Type IIS Exposed Barcode

This example illustrates a paired variant assembly method using a Type IIS exposed barcode.

A first plurality of gene fragments is synthesized comprising a barcode followed by a first restriction endonuclease site, a second restriction endonuclease site, and a first complementary determining region (CDR) segment and J segment. The CDR segment and J segment is about 100 base pairs. The first restriction endonuclease site or the second restriction endonuclease site is a Type IIS restriction endonuclease (TIIS-RE) site. A second plurality of gene fragments is synthesized comprising a first constant region followed by a self-cleaving peptide sequence, a first leader sequence, and a first variable region. The self-cleaving peptide sequence is P2A. A number of first variable regions synthesized is about 100.

The first plurality of gene fragments and the second plurality of gene fragments are combined and PCR amplified to generate a third plurality of gene fragments. The third plurality of gene fragments comprises the barcode followed by the first restriction endonuclease site, the first constant region, the cleaving peptide sequence, the first leader sequence, the first variable region, and the first CDR and J segment. The third plurality of gene fragments is combined with a fourth plurality of gene fragments comprising a vector sequence followed by a second leader sequence, a second variable region, a second CDR segment and J segment, the first TIIS-RE site, and a barcode to generate a fifth plurality of gene fragments. A number of second variable regions synthesized is about 130.

The fifth plurality of gene fragments comprises the vector sequence followed by the second leader sequence, the second variable region, the second CDR and J segment, the first TIIS-RE site, the barcode, the first TIIS-RE site, the first constant region, the cleaving peptide sequence, the first leader sequence, the first variable region, and the first CDR segment. The fifty plurality of gene fragments is PCR amplified and cloned followed by treatment with a TIIS restriction endonucleases to cut at the TIIS-RE sites to remove the barcode to generate a sixth plurality of gene fragments comprising the vector sequence followed by the second leader sequence, the second variable region, the second CDR segment, the first constant region, the cleaving peptide sequence, the first leader sequence, the first variable region, and the first CDR and J segment. The sixth plurality of gene fragments is then cloned into a vector to generate a final construct comprising the second leader sequence, the second variable region, the second CDR segment, the first constant region, the cleaving peptide sequence, the first leader sequence, the first variable region, the first CDR and J segment, and a variable constant region. A number of gene fragments synthesized is about 1000.

### Example 12. Paired Variant Assembly Using Paired Homology

This example illustrates assembly of paired variants comprising paired homology.

103 variant gene fragments comprising a first variable region are synthesized. The 103 variant gene fragments are amplified with a first CDR3 and J segment to generate a first plurality of gene fragments. A different set of 131 variant gene fragments comprising a second variable region are synthesized. The 131 variant gene fragments are amplified with a second CDR3 and J segment to generate a second plurality of gene fragments. A third plurality of 130 variant gene fragments comprising a sequence homologous to the first CDR3 and J segment followed by a constant region, a self-cleaving peptide sequence, a first leader sequence, and a 40 base pair (bp) region complementary to the second variable region is synthesized.

The first plurality of gene fragments, the second plurality of gene fragments, and the third plurality of gene fragments are assembled and cloned into a destination vector. The final construct comprises a second leader sequence followed by the second variable region, the second CDR and J segment, the second constant region, the self-cleaving peptide sequence, the first leader sequence, the first variable region, the first CDR and J segment, and the variable constant region.

### Example 13. Paired Variant Assembly Using Type IIS Sites

This example shows a paired variant assembly method of gene fragments comprising Type IIS sites.

A first plurality of gene fragments comprising a first leader sequence and a first variable region is synthesized. A second plurality of gene fragments comprising a second variable region is synthesized. A third plurality of gene fragments comprising a first Type IIS site followed by a 40 base pair (bp) region complementary to the second variable region. A fourth plurality of gene fragments comprising the 40 base pair (bp) region complementary to the second variable region followed by the second CDR3 and J segment and a variable constant segment is synthesized. A fifth plurality of gene fragments comprising a segment homologous to the first variable region followed by the first CDR3 and J segment and the TIIS site is synthesized.

The first plurality of gene fragments, the second plurality of gene fragments, the third plurality of gene fragments, the fourth plurality of gene fragments, and the fifth plurality of gene fragments are pooled and PCR amplified in order to add the first CDR3 and J segment and the second CDR3 and J segment. The resulting gene fragment comprises the second variable region followed by the second CDR3 and J segment, the TIIS site, the first variable region, and the first CDR3 and J segment. The resulting gene fragment is subject to flap endonuclease mediated nucleic acid assembly and insertion into a destination vector. The destination vector comprises the second leader sequence and the variable constant region. Following insertion into a destination vector, the gene fragment comprises the second leader sequence followed by the second variable region, the second CDR3 and J segment, the first restriction endonuclease site, the first variable region, the first CDR3 and J segment, and the variable constant region. The gene fragment is then subject to Golden Gate Assembly to insert the second constant region to generate final construct. The final construct comprises the second leader sequence followed by the second variable region, the second CDR3 and J segment, the second constant region, the self-cleaving peptide sequence, the first leader sequence, the first variable region, the first CDR3 and J segment, and the variable constant region. A number of final constructs generated is about 10000.

### Example 14. Polynucleotide Populations Specific for Each Variant

This example illustrates use of polynucleotide populations specific for each variable region. A first plurality of gene fragments is synthesized comprising a self-cleaving peptide sequence, a first leader sequence, and a first variable region. A gene fragment is synthesized comprising a segment homologous to a second variable region followed by a second CDR3 and J segment, a Type IIS site, a first CDR3 and J segment, and universal primer. The gene fragment is combined and PCR amplified with a population of gene fragments comprising a leader sequence followed by the second variable region to generate a second plurality of gene fragments comprising the second leader sequence followed by the second variable region, the second CDR3 and J segment, the Type IIS site, the first CDR3 and J segment, and the universal primer. The second plurality of gene fragments is then assembled into a destination vector comprising the second leader sequence and a variable constant region to generate a third plurality of gene fragments. The third plurality of gene fragments comprises the second leader sequence followed by the second variable region, the second CDR3 and J segment, the Type IIS site, the first CDR3 and J segment, and the variable constant region.

The first plurality of gene fragments and the third plurality of gene fragments are assembled to insert the second constant region to generate a final construct. The final construct comprises the second leader sequence followed by the second variable region, the second CDR3 and J segment, the second constant region, the self-cleaving peptide sequence, the first leader sequence, the first variable region, the first CDR3 and J segment, and the variable constant region. A number of final constructs generated is about 10000.

### Example 15. Paired Barcodes Using Dial Out PCR

This example illustrates use of paired barcodes and dial out PCR for nucleic acid assembly. A first plurality of gene fragments is synthesized comprising a first variable region. A second plurality of gene fragments is synthesized comprising a first hypervariable region followed by a 40 base pair (bp) region complementary to the first variable region, a first CDR3 and J segment, and a barcode. A third plurality of gene fragments is synthesized comprising a second leader sequence and a second variable region. A fourth plurality of gene fragments is synthesized comprising a second CDR3 and J segment. The first plurality of gene fragments and the second plurality of gene fragments are combined to create a first combinatorial library using PCR. The third plurality of gene fragments and the fourth plurality of gene fragments are combined to create a second combinatorial library using PCR.

The first combinatorial library and the second combinatorial library are assembled using flap endonuclease mediated nucleic acid assembly to generate a fifth plurality of gene fragments comprising the second leader sequence followed by the second variable region, the second CDR3 and J segment, the second constant region, the self-cleaving peptide sequence, the first leader sequence, the first variable region, the first CDR3 and J segment, the 40 base pair (bp) region complementary to a first variable region, and the barcode. The fifth plurality of gene fragments is circularized and sequenced with primers to generate a sixth plurality of gene fragments. Samples are identified by the barcode. The sixth plurality of gene fragments is then subject to dial out PCR and flap endonuclease mediated nucleic acid assembly into a vector to generate the final construct.

### Example 16. Combinatorial Assembly of Variants

Combinatorial assembly of variants was performed using methods as described herein. Four input populations (or domains) ranging from 1.2-2.2 kb in length, with 15-20 variants each were assembled (number of variants in parentheses):
5′Vector-Domain1(15)-Domain2(20)-Domain3(20)-Domain4(20)-3′Vector

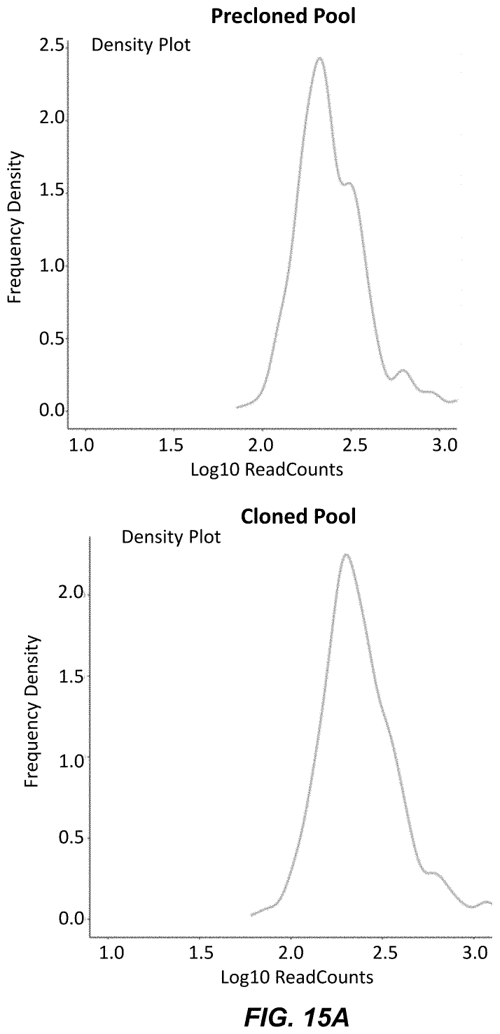

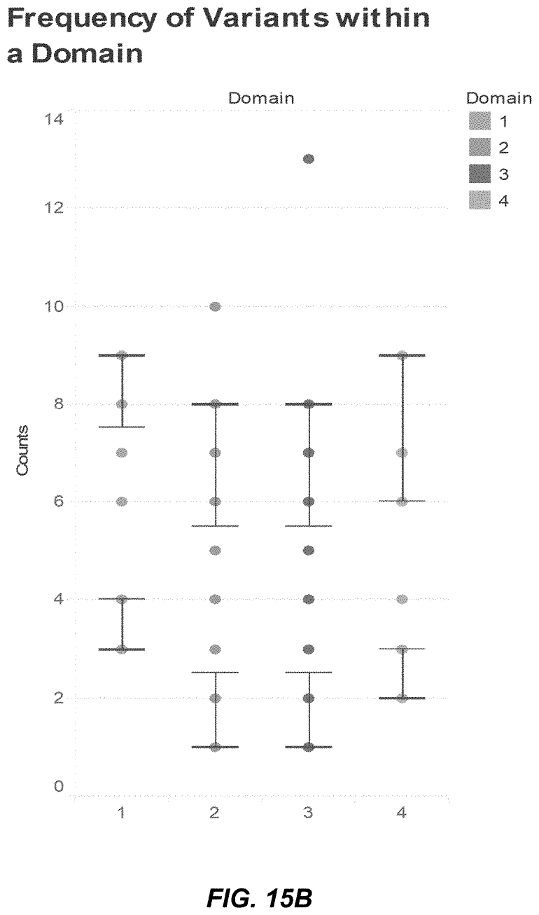

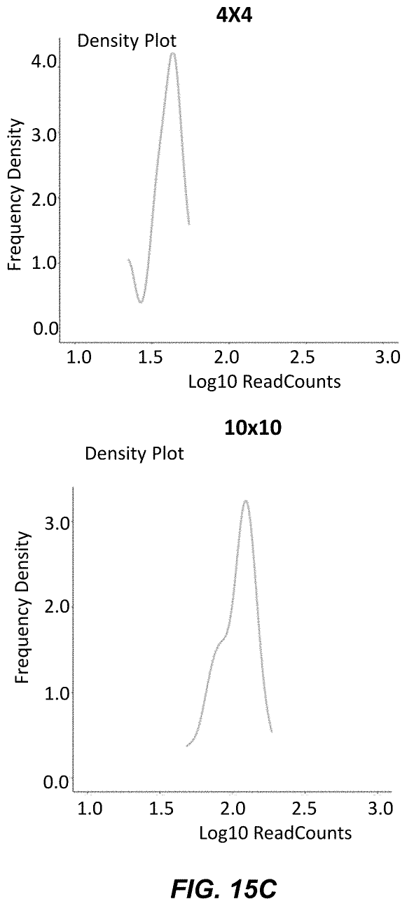

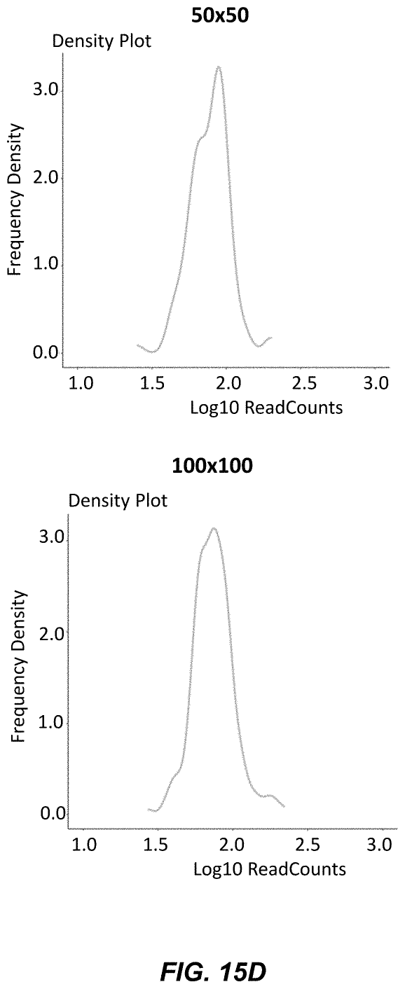

Assembly resulted in about 120000 variants and uniformity of full length sequences before and after cloning into a bacterial expression vector (FIG. 15A, Table 17) as well as uniform variant frequency (FIG. 15B).

TABLE 17

| Percentile | 90^{th}/10^{th} | 95^{th}/5^{th} |
|---|---|---|
| Metrics | Percentile | Percentile |
| Pre-Clonal Pool | 2.89 | 4.46 |
| Clonal Pool | 3.13 | 5.06 |

NGS results showed that a uniform distribution of all possible combinations of variants was obtained. This indicated the pool was unbiased with 95% of the possible variant combinations within 5× of each other. 89 individual clones were sequenced to see the different combinations present. All variants were represented in the picked colonies, and additionally all 89 pathways had a unique combination of variants (FIG. 15B).

### Example 17. Combinatorial Assembly of Variants

Combinatorial assembly of variants was performed using methods as described herein. Two input populations (or domains) approximately 1.5 kb in length, with up to 100 variants each were assembled (for number of variants X):
5′Vector-Domain1(X)-Constant Domain-Domain3(X)-3′Vector

Four pools were generated of increasing complexity (4, 10, 50, 100 variants), with up to 100,000 possible combinations. Pools showed uniform assemblies (FIG. 15C, FIG. 15D, and Table 18).

TABLE 18

| Percentile | 90^{th}/10^{th} | 95^{th}/5^{th} |
|---|---|---|
| Metrics | Percentile | Percentile |
| 4 × 4 | 1.62 | 1.99 |
| 10 × 10 | 2.00 | 2.37 |
| 50 × 50 | 2.03 | 2.61 |
| 100 × 100 | 1.99 | 2.59 |

### Example 18. Assembling a Diverse Gene Pool of 250,000 Sequences

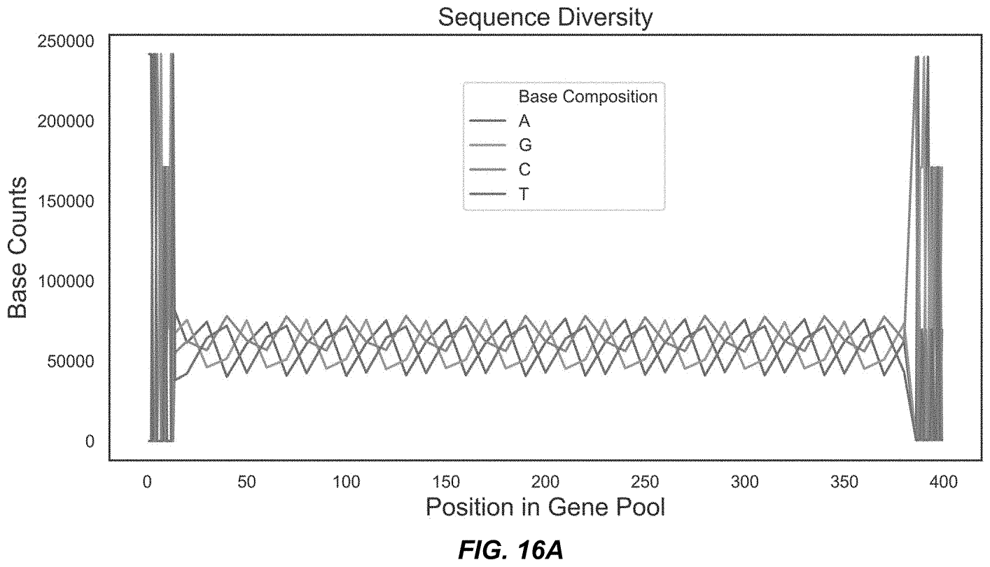

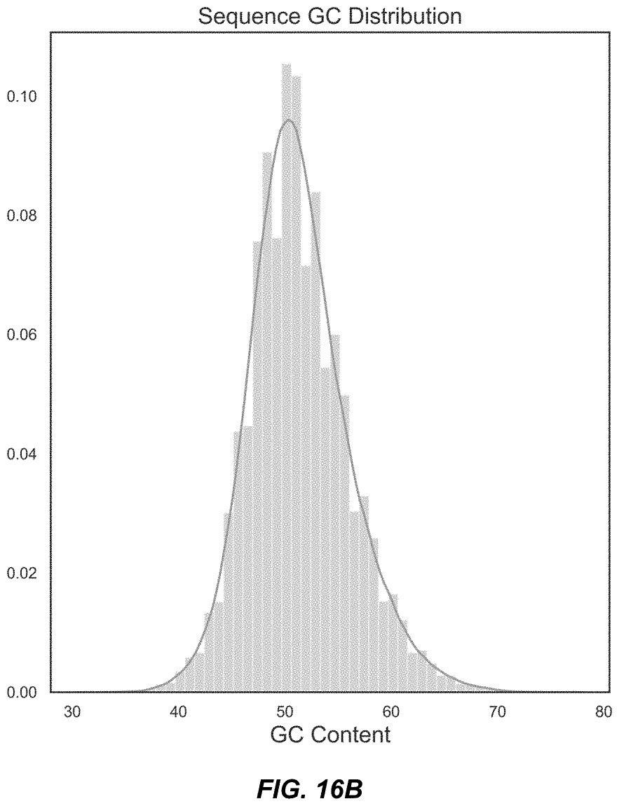

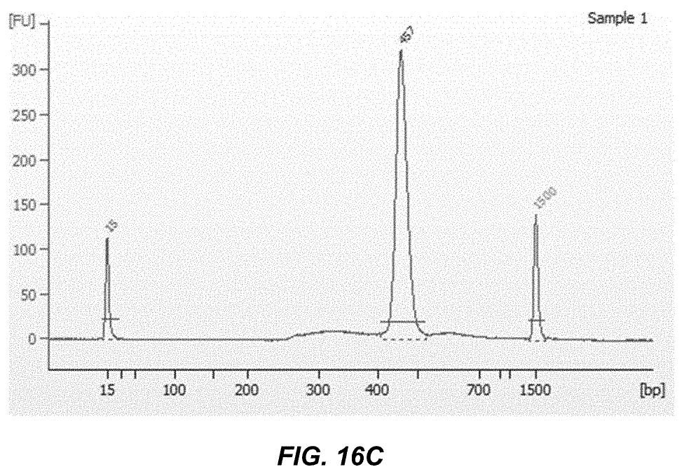

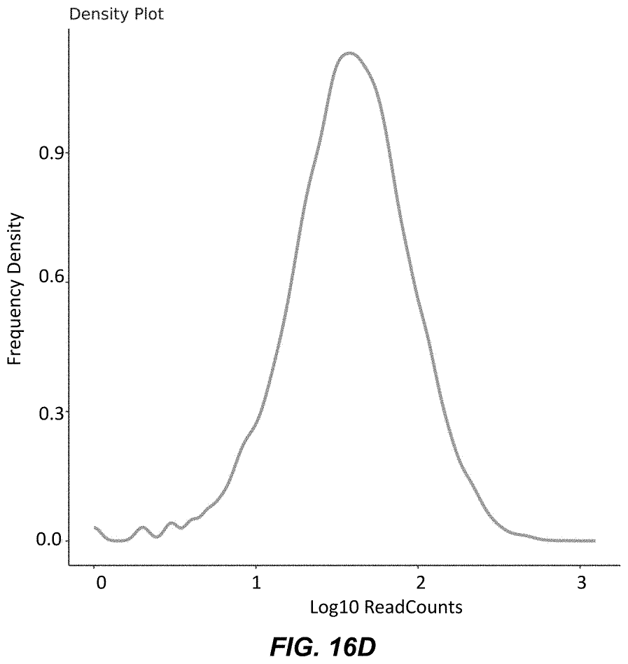

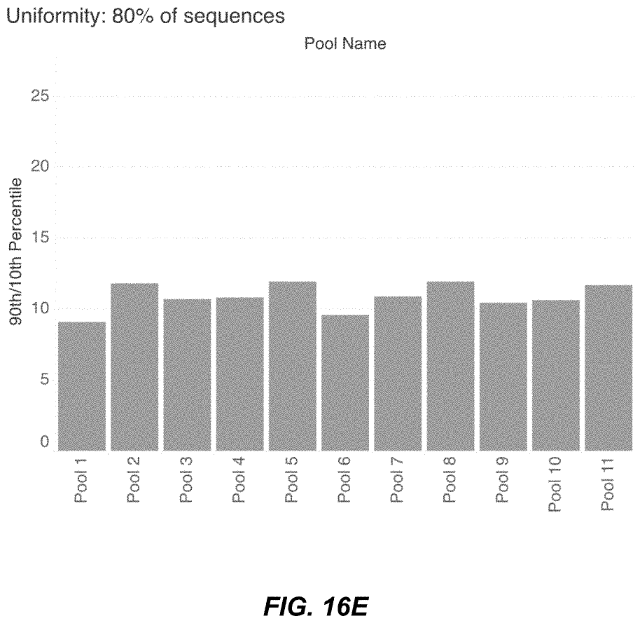

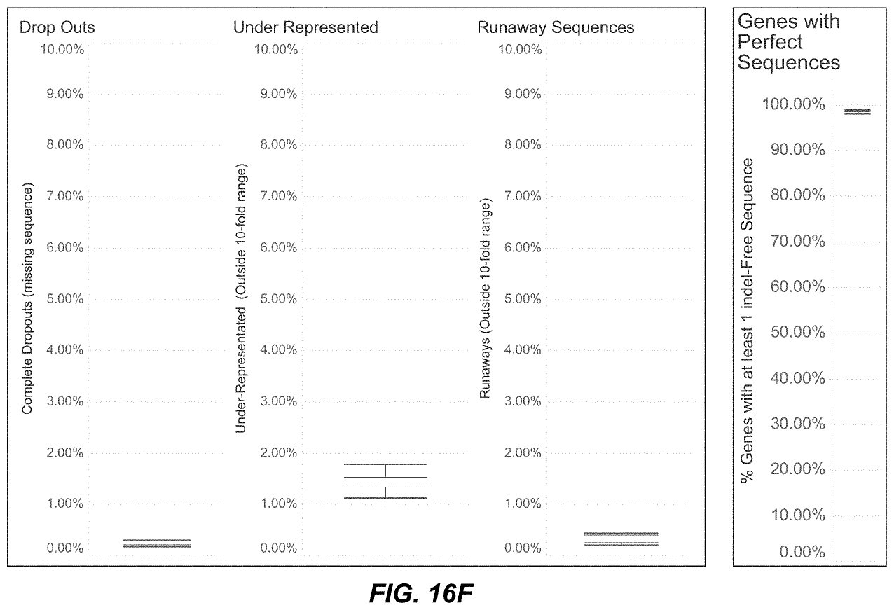

Following the general methods of Example 7, 250K sequences encoding for viral proteins were created through 11 sub gene pools. Sequences comprised viral protein DNA flanked first by a first adapter, and then a second adapter at the distal ends. 450 bp genes were distributed amongst pools by sequence diversity with an average of 23 k genes per pool (FIG. 16A and FIG. 16B. Pools were assembled and PCR amplified, and visualized with digital DNA electrophoresis (FIG. 16C). The quality of the gene pools were evaluated with normalized 50× gene coverage (FIG. 16D and FIG. 16E). 90^{th}/10^{th} Percentile Ratio indicates on average, 80% of the population lies within 10.8× of the mean. The pool was additionally characterized by drop outs (missing from pool), under represented (<10× of the mean) and runaway (>10× of the mean) (FIG. 16F). On average, >98% of genes had a detected perfect sequence at 50×NGS coverage (FIG. 16F).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What we claim is:

1. A method for nucleic acid assembly, comprising:
(a) providing a first plurality of polynucleotides, wherein each polynucleotide of the first plurality of polynucleotides comprises a first terminal region of sequence homology;
(b) providing a second plurality of polynucleotides, wherein each polynucleotide of the second plurality of polynucleotides comprises a second terminal region of sequence homology to the first terminal region of sequence homology; and
(c) contacting the first plurality of polynucleotides and the second plurality of polynucleotides with a reaction mixture comprising an exonuclease, an endonuclease, a polymerase, and a ligase to assemble a library of nucleic acids, wherein at least 80% of the nucleic acids are each present in the library in an amount within 2× of a mean frequency for each of the nucleic acids in the library.

2. The method of claim 1, wherein the first plurality of polynucleotides comprises up to 100 different sequences.

3. The method of claim 1, wherein the second plurality of polynucleotides comprises up to 100 different sequences.

4. The method of claim 1, wherein at least 10,000 nucleic acids are assembled.

5. The method of claim 1, wherein at least 100,000 nucleic acids are assembled.

6. The method of claim 1, wherein each polynucleotide of the first plurality of polynucleotides comprises up to 2500 bases in length.

7. The method of claim 1, wherein each polynucleotide of the second plurality of polynucleotides comprises up to 2500 bases in length.

8. The method of claim 1, wherein the exonuclease is exonuclease III.

9. The method of claim 1, wherein the endonuclease is a flap endonuclease.

10. The method of claim 9, wherein the flap endonuclease is flap endonuclease 1, exonuclease 1, XPG, Dna2, or GEN1.

11. The method of claim 1, wherein the polymerase comprises 5′ to 3′ polymerase activity.

12. The method of claim 1, wherein the polymerase is a DNA polymerase.

13. The method of claim 1, wherein the ligase catalyzes joining of at least two nucleic acids.

14. A method for nucleic acid assembly, comprising:
(a) de novo synthesizing a first nucleic acid comprising in 5′ to 3′ order: a barcode sequence, a first restriction endonuclease site, a second restriction endonuclease site, and a first hypervariable region sequence;
(b) de novo synthesizing a second nucleic acid comprising in 5′ to 3′ order: a first region of any defined length sequence, a self-cleaving peptide sequence, a first complementary region adjacent to a first variable region sequence, and a first variable region sequence;
(c) contacting the first nucleic acid and the second nucleic to generate a third nucleic acid;
(d) providing a fourth nucleic acid comprising in 5′ to 3′ order: a vector sequence, a second complementary region adjacent to a second variable region sequence, a second variable region sequence, a second hypervariable region sequence, the first restriction endonuclease site, and the barcode sequence;
(e) contacting the third nucleic acid and the fourth nucleic acid with a restriction endonuclease; and
(f) assembling the third nucleic acid and the fourth nucleic acid using a reaction mixture comprising one or more enzymes.

15. The method of claim 14, wherein the first restriction endonuclease site or the second restriction endonuclease site is a Type IIS restriction endonuclease (TIIS-RE) site.

16. The method of claim 14, wherein the restriction endonuclease is a Type IIS restriction endonuclease.

17. The method of claim 14, wherein the reaction mixture comprises a ligase.

18. The method of claim 14, wherein the first hypervariable region sequence and the second hypervariable region sequence each comprises a complementary determining region (CDR).

19. The method of claim 18, wherein the CDR is CDR3.

20. The method of claim 14, wherein the self-cleaving peptide is P2A.

21. The method of claim 14, wherein about 100 variants of the first variable region sequence are synthesized.

22. The method of claim 14, wherein about 130 variants of the second variable region sequence are synthesized.

23. The method of claim 14, further comprising amplifying the nucleic acid with a first primer complementary to a first barcode sequence and a second primer wherein at least 99% of the amplicons have no deletions.

24. A method for nucleic acid assembly, comprising:
(a) de novo synthesizing a first nucleic acid comprising a first variable region sequence;
(b) de novo synthesizing a second nucleic acid comprising a second variable region sequence;
(c) de novo synthesizing a third nucleic acid comprising in 5′ to 3′ order: a first region of fixed variability sequence, a first region of any defined length sequence, a self-cleaving peptide sequence, a first complementary region adjacent to a first variable region sequence, and a second region of fixed variability sequence; and
(d) contacting the first nucleic acid, the second nucleic acid, and the third nucleic acid with a reaction mixture comprising an exonuclease, an endonuclease, a polymerase, and a ligase.

25. The method of claim 24, wherein the first variable region sequence or the second variable region sequence is amplified with a hypervariable region sequence.

26. The method of claim 25, wherein the hypervariable region sequence comprises a CDR.

27. The method of claim 26, wherein the CDR is CDR3.

28. The method of claim 24, further comprising contacting with sequences comprising one or more regions of any defined length.

29. The method of claim 24, wherein about 100 variants of the first variable region sequence are synthesized.

30. The method of claim 24, wherein about 130 variants of the second variable region sequence are synthesized.

31. The method of claim 24, wherein the self-cleaving peptide is P2A.

32. The method of claim 24, wherein the exonuclease is exonuclease III.

33. The method of claim 24, wherein the endonuclease is a flap endonuclease.

34. The method of claim 33, wherein the flap endonuclease is flap endonuclease 1, exonuclease 1, XPG, Dna2, or GEN1.

35. The method of claim 24, wherein the polymerase comprises 5′ to 3′ polymerase activity.

36. The method of claim 24, wherein the polymerase is a DNA polymerase.

37. The method of claim 24, wherein the ligase catalyzes joining of at least two nucleic acids.

38. The method of claim 24, wherein the first region of fixed variability sequence and the second region of fixed variability sequence are each about 10 to about 100 base pairs.

39. The method of claim 24, wherein the first region of fixed variability sequence and the second region of fixed variability sequence are each about 40 base pairs.

40. A method for nucleic acid assembly, comprising:
(a) providing a first nucleic acid comprising a first region of any defined length sequence;
(b) providing a second nucleic acid comprising a second region of any defined length sequence;
(c) assembling a third nucleic acid comprising in 5′ to 3′ order: a first complementary region adjacent to a first variable region sequence, a first variable region sequence, and a first hypervariable region sequence;
(d) assembling a fourth nucleic acid comprising in 5′ to 3′ order: a second complementary region adjacent to a second variable region sequence, a second variable region sequence, and a second hypervariable region sequence;
(e) contacting the first nucleic acid, the second nucleic acid, the third nucleic acid, and the fourth nucleic acid; and
(f) amplifying a product from step (e).

41. The method of claim 40, further comprising an error correction step.

42. The method of claim 40, further comprising contacting a reaction mixture comprising an exonuclease, an endonuclease, a polymerase, and a ligase during step (e).

43. The method of claim 40, wherein the first hypervariable region sequence and the second hypervariable region sequence each comprises a complementary determining region (CDR).

44. The method of claim 43, wherein the CDR is CDR3.

45. The method of claim 40, wherein the first nucleic acid comprises about 300 to about 700 base pairs.

46. The method of claim 40, wherein the second nucleic acid comprises about 200 to about 600 base pairs.

47. The method of claim 40, wherein the third nucleic acid comprises about 200 to about 600 base pairs.

48. The method of claim 40, wherein the fourth nucleic acid comprises about 200 to about 600 base pairs.

49. A method for nucleic acid assembly, comprising:
(a) de novo synthesizing:
i. a first nucleic acid comprising in 5′ to 3′ order: a first complementary region adjacent to a first variable region sequence and a first variable region sequence;
ii. a second nucleic acid comprising in 5′ to 3′ order: a first region of fixed variability sequence and a first hypervariable region sequence;
iii. a third nucleic acid comprising a second variable region sequence;
iv. a fourth nucleic acid comprising in 5′ to 3′ order: a restriction endonuclease site and a second region of fixed variability sequence; and
v. a fifth nucleic acid comprising in 5′ to 3′ order: the second region of fixed variability sequence, a second hypervariable region sequence, and a variable constant region sequence;
(b) contacting the first nucleic acid, the second nucleic acid, the third nucleic acid, the fourth nucleic acid, and the fifth nucleic acid with a reaction mixture comprising an exonuclease, an endonuclease, a polymerase, and a ligase; and
(c) cloning a construct of step (b) into a vector sequence.

50. The method of claim 49, wherein the first hypervariable region sequence and the second hypervariable region sequence each comprises a complementary determining region (CDR).

51. The method of claim 49, wherein the CDR is CDR3.

52. The method of claim 49, further comprising contacting one or more variable constant regions.

53. The method of claim 49, wherein the exonuclease is exonuclease III.

54. The method of claim 49, wherein the endonuclease is a flap endonuclease.

55. The method of claim 54, wherein the flap endonuclease is flap endonuclease 1, exonuclease 1, XPG, Dna2, or GEN1.

56. The method of claim 49, wherein the polymerase comprises 5′ to 3′ polymerase activity.

57. A method for nucleic acid assembly, comprising:
(a) providing a first nucleic acid comprising in 5′ to 3′ order: a first complementary region adjacent to a first variable region sequence and a first variable region sequence;
(b) providing a second nucleic acid sequence comprising in 5′ to 3′ order: a first region of fixed variability sequence, a first hypervariable region sequence, a restriction endonuclease site, a second hypervariable region sequence, and a universal primer;
(c) amplifying the first nucleic acid and the second nucleic acid to generate a third nucleic acid;
(d) providing a vector sequence comprising the first complementary region adjacent to the first variable region sequence and a first region of any defined length sequence;
(e) contacting the third nucleic acid and the vector sequence;
(f) contacting a fourth nucleic acid comprising in 5′ to 3′ order: a self-cleaving peptide sequence, a second complementary region adjacent to a second variable region sequence, and a second variable region sequence.

58. The method of claim 57, wherein the first hypervariable region sequence and the second hypervariable region sequence each comprises a complementary determining region (CDR).

59. The method of claim 58, wherein the CDR is CDR3.

60. The method of claim 57, wherein the self-cleaving peptide is P2A.

61. A method for nucleic acid assembly, comprising:
(a) de novo synthesizing:
i. a first nucleic acid comprising a first complementary region adjacent to a first variable region sequence and a first variable region sequence;
ii. a second nucleic acid comprising a first hypervariable region sequence;
iii. a third nucleic acid comprising a second variable region sequence;
iv. a fourth nucleic acid comprising in 5′ to 3′ order: a first hypervariable region sequence, a first region of fixed variability, and a barcode;
(b) amplifying the first nucleic acid and the second nucleic acid to generate a fifth nucleic acid;
(c) amplifying the third nucleic acid and the fourth nucleic acid to generate a fifth nucleic acid;
(d) contacting the fifth nucleic acid and the sixth nucleic acid with a reaction mixture comprising an exonuclease, an endonuclease, a polymerase, and a ligase to generate a seventh nucleic acid;
(e) circularizing the seventh nucleic acid;
(f) sequencing and identifying the seventh nucleic acid using the barcode;
(g) amplifying the seventh nucleic acid; and
(h) assembling the seventh nucleic acid in a vector using the reaction mixture comprising the exonuclease, the endonuclease, the polymerase, and the ligase.

62. The method of claim 61, wherein the first variable region sequence or the second variable region sequence is amplified with a hypervariable region sequence.

63. The method of claim 62, wherein the hypervariable region sequence comprises a CDR.

64. The method of claim 63, wherein the CDR is CDR3.

65. The method of claim 61, further comprising contacting with sequences comprising one or more regions of any defined length.

66. The method of claim 61, wherein about 100 variants of the first variable region sequence are synthesized.

67. The method of claim 61, wherein about 130 variants of the second variable region sequence are synthesized.

68. The method of claim 61, wherein the self-cleaving peptide is P2A.

69. The method of claim 61, wherein the exonuclease is exonuclease III.

70. The method of claim 61, wherein the endonuclease is a flap endonuclease.

71. The method of claim 70, wherein the flap endonuclease is flap endonuclease 1, exonuclease 1, XPG, Dna2, or GEN1.

72. The method of claim 61, wherein the polymerase comprises 5′ to 3′ polymerase activity.

73. The method of claim 61, wherein the polymerase is a DNA polymerase.

74. The method of claim 61, wherein the ligase catalyzes joining of at least two nucleic acids.

75. The method of claim 61, wherein the first region of fixed variability sequence and the second region of fixed variability sequence are each about 10 to about 100 base pairs.

76. The method of claim 61, wherein the first region of fixed variability sequence and the second region of fixed variability sequence are each about 40 base pairs.


SEQUENCE LISTING (4 sequences)

SEQ ID NO: 1 - DNA, 62 bp, Artificial Sequence
  Description of Artificial Sequence Synthetic oligonucleotide
  modified_base (51)..(52): Thymidine-succinyl hexamide CED phosphoramidite
agacaatcaaccatttggggtggacagccttgacctctagacttcggcattttttttttttt

SEQ ID NO: 2 - DNA, 112 bp, Artificial Sequence
  Description of Artificial Sequence Synthetic polynucleotide
  modified_base (101)..(102): Thymidine-succinyl hexamide CED phosphoramidite
cgggatccttatcgtcatcgtcgtacagatcccgacccatttgctgtccaccagtcatgctagccataccatgatgatgatgatgatgagaaccccgcattttttttttttt

SEQ ID NO: 3 - DNA, 19 bp, Artificial Sequence
  Description of Artificial Sequence Synthetic primer
atgcggggttctcatcatc

SEQ ID NO: 4 - DNA, 20 bp, Artificial Sequence
  Description of Artificial Sequence Synthetic primer
cgggatccttatcgtcatcg